(19) United States
(12) Patent Application Publication
(10) Pub. No.: US 2025/0378946 A1
(43) Pub. Date: Dec. 11, 2025

(54) PREGNANCY USER INTERFACES

(71) Applicant: Apple Inc., Cupertino, CA (US)

(72) Inventors: Lindsey MARATTA, Sunnyvale, CA (US); Dima BADAWI, San Jose, CA (US); Niharika BEDEKAR, San Francisco, CA (US); Christine L. CURRY, Sunnyvale, CA (US); Nicholas D. FELTON, Sunnyvale, CA (US); Andrea FIEGER, Menlo Park, CA (US); Charmian B. NAGUIT, San Rafael, CA (US); Helen RODEMANN, Palo Alto, CA (US)

(21) Appl. No.: 19/186,518

(22) Filed: Apr. 22, 2025

Related U.S. Application Data
(60) Provisional application No. 63/657,913, filed on Jun. 9, 2024.

(51) Int. Cl. G16H 40/67; A61B 5/00; A61B 5/0205; A61B 5/11; A61B 5/16; G06F 3/0481; G06F 3/04847

(52) U.S. Cl. CPC G16H 40/67; A61B 5/0205; A61B 5/1123; A61B 5/165; A61B 5/4343; A61B 5/7275; A61B 5/742; G06F 3/0481; G06F 3/04847

(57) ABSTRACT

The present disclosure generally relates to pregnancy user interfaces. In some examples, a computer system displays one or more user interfaces for customizing notifications related to a pregnancy event after receiving a request to add the pregnancy event to an account of the computer system. In some examples, a computer system displays cycle tracking indicators corresponding to respective days with different appearances based on whether an account of the computer system includes an indication that a user of the account is pregnant. In some examples, a computer system displays a graphical representation of first health data corresponding to a first duration and, based on a determination that an account of the computer system includes an indication that a user is pregnant, an indication that a difference between second health data corresponding to a second duration and the first health information is based on the user being pregnant.

### CROSS-REFERENCE TO RELATED APPLICATIONS

This application claims priority to U.S. Patent Application No. 63/657,913, entitled “PREGNANCY USER INTERFACES,” filed on Jun. 9, 2024, the content of which is hereby incorporated by reference in its entirety.

### FIELD

The present disclosure relates generally to computer user interfaces, and more specifically to techniques for tracking pregnancy events.

### BACKGROUND

Recurring (e.g., reoccurring) events, such as health events and/or pregnancies can be tracked on electronic devices to log past events and/or to predict future events.

### BRIEF SUMMARY

Some techniques for tracking pregnancy events using electronic devices, however, are generally cumbersome and inefficient. For example, some existing techniques use a complex and time-consuming user interface, which may include multiple key presses or keystrokes. Existing techniques require more time than necessary, wasting user time and device energy. This latter consideration is particularly important in battery-operated devices.

Accordingly, the present technique provides electronic devices with faster, more efficient methods and interfaces for tracking pregnancy events. Such methods and interfaces optionally complement or replace other methods for tracking pregnancy events. Such methods and interfaces reduce the cognitive burden on a user and produce a more efficient human-machine interface. For battery-operated computing devices, such methods and interfaces conserve power and increase the time between battery charges.

A method is described, in accordance with some embodiments. The method is performed at a computer system that is in communication with one or more display generation components and one or more input devices. The method comprises: detecting, via the one or more input devices, one or more user inputs corresponding to a request to add a pregnancy event to health information of an account of the computer system; in response to detecting the one or more user inputs corresponding to the request to add the pregnancy event to the health information of the account of the computer system, displaying, via the one or more display generation components, a pregnancy user interface that includes a pregnancy estimation user interface object; detecting, via the one or more input devices, one or more user inputs corresponding to the pregnancy estimation user interface object; in response to detecting the one or more user inputs corresponding to the pregnancy estimation user interface object, receiving information about an age of the pregnancy event; and after receiving the information about the age of the pregnancy event, displaying, via the one or more display generation components, one or more user interfaces corresponding to options to configure respective notifications related to the pregnancy event.

A non-transitory computer-readable storage medium is described, in accordance with some embodiments. The non-transitory computer-readable storage medium stores one or more programs configured to be executed by one or more processors of a computer system that is in communication with one or more display generation components and one or more input devices, the one or more programs including instructions for: detecting, via the one or more input devices, one or more user inputs corresponding to a request to add a pregnancy event to health information of an account of the computer system; in response to detecting the one or more user inputs corresponding to the request to add the pregnancy event to the health information of the account of the computer system, displaying, via the one or more display generation components, a pregnancy user interface that includes a pregnancy estimation user interface object; detecting, via the one or more input devices, one or more user inputs corresponding to the pregnancy estimation user interface object; in response to detecting the one or more user inputs corresponding to the pregnancy estimation user interface object, receiving information about an age of the pregnancy event; and after receiving the information about the age of the pregnancy event, displaying, via the one or more display generation components, one or more user interfaces corresponding to options to configure respective notifications related to the pregnancy event.

A transitory computer-readable storage medium is described, in accordance with some embodiments. The transitory computer-readable storage medium stores one or more programs configured to be executed by one or more processors of a computer system that is in communication with one or more display generation components and one or more input devices, the one or more programs including instructions for: detecting, via the one or more input devices, one or more user inputs corresponding to a request to add a pregnancy event to health information of an account of the computer system; in response to detecting the one or more user inputs corresponding to the request to add the pregnancy event to the health information of the account of the computer system, displaying, via the one or more display generation components, a pregnancy user interface that includes a pregnancy estimation user interface object; detecting, via the one or more input devices, one or more user inputs corresponding to the pregnancy estimation user interface object; in response to detecting the one or more user inputs corresponding to the pregnancy estimation user interface object, receiving information about an age of the pregnancy event; and after receiving the information about the age of the pregnancy event, displaying, via the one or more display generation components, one or more user interfaces corresponding to options to configure respective notifications related to the pregnancy event.

A computer system is described, in accordance with some embodiments. The computer system is configured to communicate with one or more display generation components and one or more input devices and comprises: one or more processors; and memory storing one or more programs configured to be executed by the one or more processors, the one or more programs including instructions for: detecting, via the one or more input devices, one or more user inputs corresponding to a request to add a pregnancy event to health information of an account of the computer system; in response to detecting the one or more user inputs corresponding to the request to add the pregnancy event to the health information of the account of the computer system, displaying, via the one or more display generation components, a pregnancy user interface that includes a pregnancy estimation user interface object; detecting, via the one or more input devices, one or more user inputs corresponding to the pregnancy estimation user interface object; in response to detecting the one or more user inputs corresponding to the pregnancy estimation user interface object, receiving information about an age of the pregnancy event; and after receiving the information about the age of the pregnancy event, displaying, via the one or more display generation components, one or more user interfaces corresponding to options to configure respective notifications related to the pregnancy event.

A computer system is described, in accordance with some embodiments. The computer system is configured to communicate with one or more display generation components and one or more input devices and comprises: means for detecting, via the one or more input devices, one or more user inputs corresponding to a request to add a pregnancy event to health information of an account of the computer system; means for, in response to detecting the one or more user inputs corresponding to the request to add the pregnancy event to the health information of the account of the computer system, displaying, via the one or more display generation components, a pregnancy user interface that includes a pregnancy estimation user interface object; means for detecting, via the one or more input devices, one or more user inputs corresponding to the pregnancy estimation user interface object; means for, in response to detecting the one or more user inputs corresponding to the pregnancy estimation user interface object, receiving information about an age of the pregnancy event; and means for, after receiving the information about the age of the pregnancy event, displaying, via the one or more display generation components, one or more user interfaces corresponding to options to configure respective notifications related to the pregnancy event.

A computer program product is described, in accordance with some embodiments. The computer program product comprises one or more programs configured to be executed by one or more processors of a computer system that is in communication with one or more display generation components and one or more input devices, the one or more programs including instructions for: detecting, via the one or more input devices, one or more user inputs corresponding to a request to add a pregnancy event to health information of an account of the computer system; in response to detecting the one or more user inputs corresponding to the request to add the pregnancy event to the health information of the account of the computer system, displaying, via the one or more display generation components, a pregnancy user interface that includes a pregnancy estimation user interface object; detecting, via the one or more input devices, one or more user inputs corresponding to the pregnancy estimation user interface object; in response to detecting the one or more user inputs corresponding to the pregnancy estimation user interface object, receiving information about an age of the pregnancy event; and after receiving the information about the age of the pregnancy event, displaying, via the one or more display generation components, one or more user interfaces corresponding to options to configure respective notifications related to the pregnancy event.

A method is described, in accordance with some embodiments. The method is performed at a computer system that is in communication with one or more display generation components and one or more input devices. The method comprises: detecting, via the one or more input devices, user input corresponding to a request to display a cycle tracking user interface; and in response to detecting the user input corresponding to the request to display the cycle tracking user interface, displaying, via the one or more display generation components, the cycle tracking user interface, wherein the cycle tracking user interface includes: a first cycle tracking indicator corresponding to a first day; and a second cycle tracking indicator corresponding to a second day that is different from the first day; wherein: in accordance with a determination that an account corresponding to the cycle tracking user interface includes an indication that a user of the account is pregnant on the first day, the first cycle tracking indicator has a first appearance; in accordance with a determination that the account corresponding to the cycle tracking user interface does not include an indication that the user of the account is pregnant on the first day, the first cycle tracking indicator has a second appearance that is different from the first appearance; in accordance with a determination that the account corresponding to the cycle tracking user interface includes an indication that the user of the account is pregnant on the second day, the second cycle tracking indicator has the first appearance; and in accordance with a determination that the account corresponding to the cycle tracking user interface does not include an indication that the user of the account is pregnant on the second day, the second cycle tracking indicator has the second appearance that is different from the first appearance.

A non-transitory computer-readable storage medium is described, in accordance with some embodiments. The non-transitory computer-readable storage medium stores one or more programs configured to be executed by one or more processors of a computer system that is in communication with one or more display generation components and one or more input devices, the one or more programs including instructions for: detecting, via the one or more input devices, user input corresponding to a request to display a cycle tracking user interface; and in response to detecting the user input corresponding to the request to display the cycle tracking user interface, displaying, via the one or more display generation components, the cycle tracking user interface, wherein the cycle tracking user interface includes: a first cycle tracking indicator corresponding to a first day; and a second cycle tracking indicator corresponding to a second day that is different from the first day; wherein: in accordance with a determination that an account corresponding to the cycle tracking user interface includes an indication that a user of the account is pregnant on the first day, the first cycle tracking indicator has a first appearance; in accordance with a determination that the account corresponding to the cycle tracking user interface does not include an indication that the user of the account is pregnant on the first day, the first cycle tracking indicator has a second appearance that is different from the first appearance; in accordance with a determination that the account corresponding to the cycle tracking user interface includes an indication that the user of the account is pregnant on the second day, the second cycle tracking indicator has the first appearance; and in accordance with a determination that the account corresponding to the cycle tracking user interface does not include an indication that the user of the account is pregnant on the second day, the second cycle tracking indicator has the second appearance that is different from the first appearance.

A transitory computer-readable storage medium is described, in accordance with some embodiments. The transitory computer-readable storage medium stores one or more programs configured to be executed by one or more processors of a computer system that is in communication with one or more display generation components and one or more input devices, the one or more programs including instructions for: detecting, via the one or more input devices, user input corresponding to a request to display a cycle tracking user interface; and in response to detecting the user input corresponding to the request to display the cycle tracking user interface, displaying, via the one or more display generation components, the cycle tracking user interface, wherein the cycle tracking user interface includes: a first cycle tracking indicator corresponding to a first day; and a second cycle tracking indicator corresponding to a second day that is different from the first day; wherein: in accordance with a determination that an account corresponding to the cycle tracking user interface includes an indication that a user of the account is pregnant on the first day, the first cycle tracking indicator has a first appearance; in accordance with a determination that the account corresponding to the cycle tracking user interface does not include an indication that the user of the account is pregnant on the first day, the first cycle tracking indicator has a second appearance that is different from the first appearance; in accordance with a determination that the account corresponding to the cycle tracking user interface includes an indication that the user of the account is pregnant on the second day, the second cycle tracking indicator has the first appearance; and in accordance with a determination that the account corresponding to the cycle tracking user interface does not include an indication that the user of the account is pregnant on the second day, the second cycle tracking indicator has the second appearance that is different from the first appearance.

A computer system is described, in accordance with some embodiments. The computer system is configured to communicate with one or more display generation components and one or more input devices and comprises: one or more processors; and memory storing one or more programs configured to be executed by the one or more processors, the one or more programs including instructions for: detecting, via the one or more input devices, user input corresponding to a request to display a cycle tracking user interface; and in response to detecting the user input corresponding to the request to display the cycle tracking user interface, displaying, via the one or more display generation components, the cycle tracking user interface, wherein the cycle tracking user interface includes: a first cycle tracking indicator corresponding to a first day; and a second cycle tracking indicator corresponding to a second day that is different from the first day; wherein: in accordance with a determination that an account corresponding to the cycle tracking user interface includes an indication that a user of the account is pregnant on the first day, the first cycle tracking indicator has a first appearance; in accordance with a determination that the account corresponding to the cycle tracking user interface does not include an indication that the user of the account is pregnant on the first day, the first cycle tracking indicator has a second appearance that is different from the first appearance; in accordance with a determination that the account corresponding to the cycle tracking user interface includes an indication that the user of the account is pregnant on the second day, the second cycle tracking indicator has the first appearance; and in accordance with a determination that the account corresponding to the cycle tracking user interface does not include an indication that the user of the account is pregnant on the second day, the second cycle tracking indicator has the second appearance that is different from the first appearance.

A computer system is described, in accordance with some embodiments. The computer system is configured to communicate with one or more display generation components and one or more input devices and comprises: means for detecting, via the one or more input devices, user input corresponding to a request to display a cycle tracking user interface; and means for, in response to detecting the user input corresponding to the request to display the cycle tracking user interface, displaying, via the one or more display generation components, the cycle tracking user interface, wherein the cycle tracking user interface includes: a first cycle tracking indicator corresponding to a first day; and a second cycle tracking indicator corresponding to a second day that is different from the first day; wherein: in accordance with a determination that an account corresponding to the cycle tracking user interface includes an indication that a user of the account is pregnant on the first day, the first cycle tracking indicator has a first appearance; in accordance with a determination that the account corresponding to the cycle tracking user interface does not include an indication that the user of the account is pregnant on the first day, the first cycle tracking indicator has a second appearance that is different from the first appearance; in accordance with a determination that the account corresponding to the cycle tracking user interface includes an indication that the user of the account is pregnant on the second day, the second cycle tracking indicator has the first appearance; and in accordance with a determination that the account corresponding to the cycle tracking user interface does not include an indication that the user of the account is pregnant on the second day, the second cycle tracking indicator has the second appearance that is different from the first appearance.

A computer program product is described, in accordance with some embodiments. The computer program product comprises one or more programs configured to be executed by one or more processors of a computer system that is in communication with one or more display generation components and one or more input devices, the one or more programs including instructions for: detecting, via the one or more input devices, user input corresponding to a request to display a cycle tracking user interface; and in response to detecting the user input corresponding to the request to display the cycle tracking user interface, displaying, via the one or more display generation components, the cycle tracking user interface, wherein the cycle tracking user interface includes: a first cycle tracking indicator corresponding to a first day; and a second cycle tracking indicator corresponding to a second day that is different from the first day; wherein: in accordance with a determination that an account corresponding to the cycle tracking user interface includes an indication that a user of the account is pregnant on the first day, the first cycle tracking indicator has a first appearance; in accordance with a determination that the account corresponding to the cycle tracking user interface does not include an indication that the user of the account is pregnant on the first day, the first cycle tracking indicator has a second appearance that is different from the first appearance; in accordance with a determination that the account corresponding to the cycle tracking user interface includes an indication that the user of the account is pregnant on the second day, the second cycle tracking indicator has the first appearance; and in accordance with a determination that the account corresponding to the cycle tracking user interface does not include an indication that the user of the account is pregnant on the second day, the second cycle tracking indicator has the second appearance that is different from the first appearance.

A method is described, in accordance with some embodiments. The method is performed at a computer system that is in communication with one or more display generation components and one or more input devices. The method comprises: detecting, via the one or more input devices, user input corresponding to a request to display a user interface corresponding to health information of an account of the computer system; and in response to detecting the user input corresponding to the request to display the user interface corresponding to health information of the account of the computer system, displaying, via the one or more display generation components, the user interface corresponding to health information of the account of the computer system, wherein displaying the user interface includes: displaying, via the one or more display generation components, a graphical representation of first health data corresponding to a first time duration; and in accordance with a determination that the account of the computer system includes an indication that a user of the account is pregnant, displaying, via the one or more display generation components, an indication that a difference between second health data and the first health data is at least partially based on the user of the account being pregnant, wherein the second health data corresponds to a second time duration that is after the first time duration.

A non-transitory computer-readable storage medium is described, in accordance with some embodiments. The non-transitory computer-readable storage medium stores one or more programs configured to be executed by one or more processors of a computer system that is in communication with one or more display generation components and one or more input devices, the one or more programs including instructions for: detecting, via the one or more input devices, user input corresponding to a request to display a user interface corresponding to health information of an account of the computer system; and in response to detecting the user input corresponding to the request to display the user interface corresponding to health information of the account of the computer system, displaying, via the one or more display generation components, the user interface corresponding to health information of the account of the computer system, wherein displaying the user interface includes: displaying, via the one or more display generation components, a graphical representation of first health data corresponding to a first time duration; and in accordance with a determination that the account of the computer system includes an indication that a user of the account is pregnant, displaying, via the one or more display generation components, an indication that a difference between second health data and the first health data is at least partially based on the user of the account being pregnant, wherein the second health data corresponds to a second time duration that is after the first time duration.

A transitory computer-readable storage medium is described, in accordance with some embodiments. The transitory computer-readable storage medium stores one or more programs configured to be executed by one or more processors of a computer system that is in communication with one or more display generation components and one or more input devices, the one or more programs including instructions for: detecting, via the one or more input devices, user input corresponding to a request to display a user interface corresponding to health information of an account of the computer system; and in response to detecting the user input corresponding to the request to display the user interface corresponding to health information of the account of the computer system, displaying, via the one or more display generation components, the user interface corresponding to health information of the account of the computer system, wherein displaying the user interface includes: displaying, via the one or more display generation components, a graphical representation of first health data corresponding to a first time duration; and in accordance with a determination that the account of the computer system includes an indication that a user of the account is pregnant, displaying, via the one or more display generation components, an indication that a difference between second health data and the first health data is at least partially based on the user of the account being pregnant, wherein the second health data corresponds to a second time duration that is after the first time duration.

A computer system is described, in accordance with some embodiments. The computer system is configured to communicate with one or more display generation components and one or more input devices and comprises: one or more processors; and memory storing one or more programs configured to be executed by the one or more processors, the one or more programs including instructions for: detecting, via the one or more input devices, user input corresponding to a request to display a user interface corresponding to health information of an account of the computer system; and in response to detecting the user input corresponding to the request to display the user interface corresponding to health information of the account of the computer system, displaying, via the one or more display generation components, the user interface corresponding to health information of the account of the computer system, wherein displaying the user interface includes: displaying, via the one or more display generation components, a graphical representation of first health data corresponding to a first time duration; and in accordance with a determination that the account of the computer system includes an indication that a user of the account is pregnant, displaying, via the one or more display generation components, an indication that a difference between second health data and the first health data is at least partially based on the user of the account being pregnant, wherein the second health data corresponds to a second time duration that is after the first time duration.

A computer system is described, in accordance with some embodiments. The computer system is configured to communicate with one or more display generation components and one or more input devices and comprises: means for detecting, via the one or more input devices, user input corresponding to a request to display a user interface corresponding to health information of an account of the computer system; and means for, in response to detecting the user input corresponding to the request to display the user interface corresponding to health information of the account of the computer system, displaying, via the one or more display generation components, the user interface corresponding to health information of the account of the computer system, wherein displaying the user interface includes: displaying, via the one or more display generation components, a graphical representation of first health data corresponding to a first time duration; and in accordance with a determination that the account of the computer system includes an indication that a user of the account is pregnant, displaying, via the one or more display generation components, an indication that a difference between second health data and the first health data is at least partially based on the user of the account being pregnant, wherein the second health data corresponds to a second time duration that is after the first time duration.

A computer program product is described, in accordance with some embodiments. The computer program product comprises one or more programs configured to be executed by one or more processors of a computer system that is in communication with one or more display generation components and one or more input devices, the one or more programs including instructions for: detecting, via the one or more input devices, user input corresponding to a request to display a user interface corresponding to health information of an account of the computer system; and in response to detecting the user input corresponding to the request to display the user interface corresponding to health information of the account of the computer system, displaying, via the one or more display generation components, the user interface corresponding to health information of the account of the computer system, wherein displaying the user interface includes: displaying, via the one or more display generation components, a graphical representation of first health data corresponding to a first time duration; and in accordance with a determination that the account of the computer system includes an indication that a user of the account is pregnant, displaying, via the one or more display generation components, an indication that a difference between second health data and the first health data is at least partially based on the user of the account being pregnant, wherein the second health data corresponds to a second time duration that is after the first time duration.

Executable instructions for performing these functions are, optionally, included in a non-transitory computer-readable storage medium or other computer program product configured for execution by one or more processors. Executable instructions for performing these functions are, optionally, included in a transitory computer-readable storage medium or other computer program product configured for execution by one or more processors.

Thus, devices are provided with faster, more efficient methods and interfaces for tracking pregnancy events, thereby increasing the effectiveness, efficiency, and user satisfaction with such devices. Such methods and interfaces may complement or replace other methods for tracking pregnancy events.

### DESCRIPTION OF THE FIGURES

For a better understanding of the various described embodiments, reference should be made to the Description of Embodiments below, in conjunction with the following drawings in which like reference numerals refer to corresponding parts throughout the figures.

FIG. 1A is a block diagram illustrating a portable multifunction device with a touch-sensitive display in accordance with some embodiments.

FIG. 1B is a block diagram illustrating exemplary components for event handling in accordance with some embodiments.

FIG. 2 illustrates a portable multifunction device having a touch screen in accordance with some embodiments.

FIG. 3A is a block diagram of an exemplary multifunction device with a display and a touch-sensitive surface in accordance with some embodiments.

FIGS. 3B-3G illustrate the use of Application Programming Interfaces (APIs) to perform operations in accordance with some embodiments.

FIG. 4A illustrates an exemplary user interface for a menu of applications on a portable multifunction device in accordance with some embodiments.

FIG. 4B illustrates an exemplary user interface for a multifunction device with a touch-sensitive surface that is separate from the display in accordance with some embodiments.

FIG. 5A illustrates a personal electronic device in accordance with some embodiments.

FIG. 5B is a block diagram illustrating a personal electronic device in accordance with some embodiments.

FIGS. 6A-6W illustrate exemplary user interfaces for managing notifications related to a pregnancy event, in accordance with some embodiments.

FIG. 7 is a flow diagram illustrating a method for managing notifications related to a pregnancy event, in accordance with some embodiments.

FIGS. 8A-8L illustrate exemplary user interfaces for tracking a pregnancy event, in accordance with some embodiments.

FIG. 9 is a flow diagram illustrating a method for tracking a pregnancy event, in accordance with some embodiments.

FIGS. 10A-10P illustrate exemplary user interfaces for tracking health information during a pregnancy event, in accordance with some embodiments.

FIG. 11 is a flow diagram illustrating a method for tracking health information during a pregnancy event, in accordance with some embodiments.

### DESCRIPTION OF EMBODIMENTS

The following description sets forth exemplary methods, parameters, and the like. It should be recognized, however, that such description is not intended as a limitation on the scope of the present disclosure but is instead provided as a description of exemplary embodiments.

There is a need for electronic devices that provide efficient methods and interfaces for tracking pregnancy events. For example, there is a need for electronic devices that facilitate a user's ability to customize notifications after and/or in response to providing health information, such as information related to a pregnancy event. There is also a need for electronic devices that efficiently provide tracking information related to a pregnancy event. Further still, there is a need for electronic devices that display health and/or activity information in different ways based on whether or not a user of the electronic device has indicated that they are pregnant. Such techniques can reduce the cognitive burden on a user who track pregnancy events, thereby enhancing productivity. Further, such techniques can reduce processor and battery power otherwise wasted on redundant user inputs.

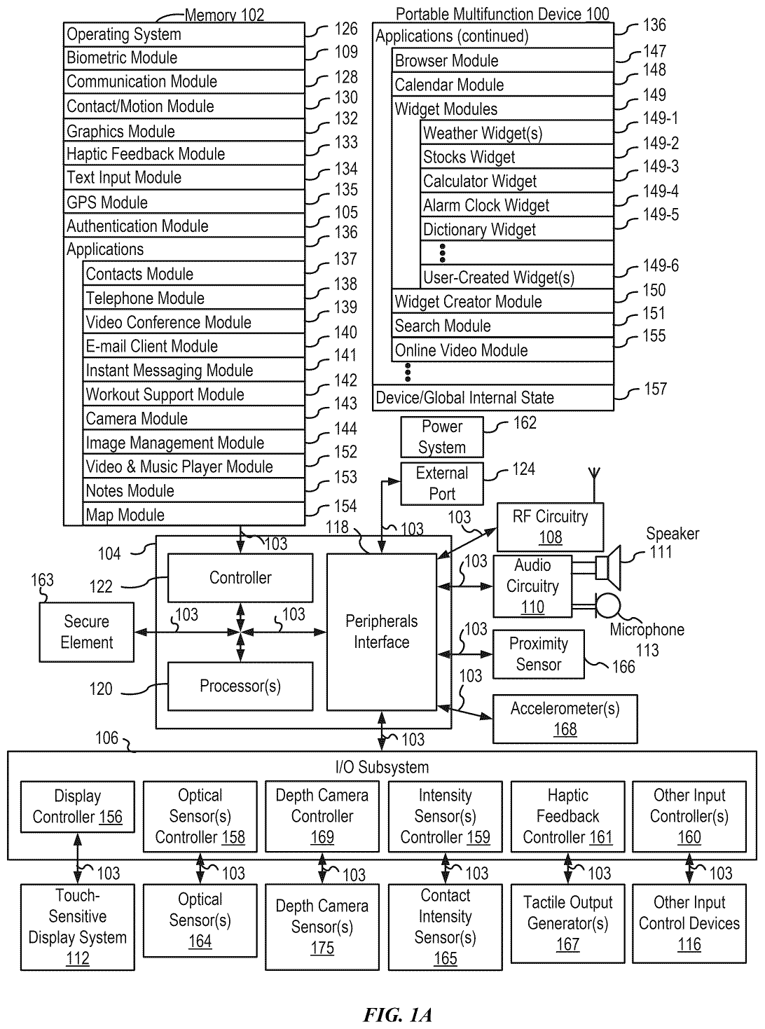

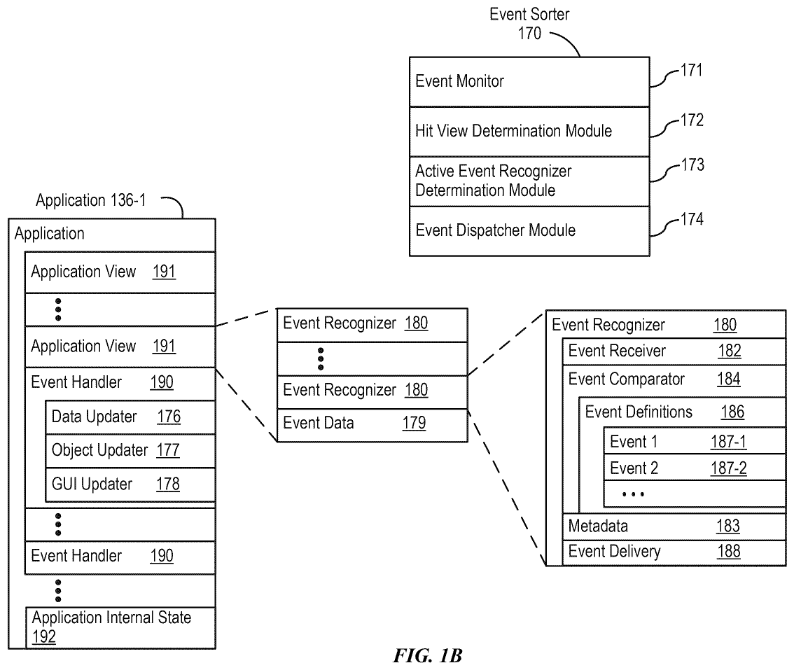

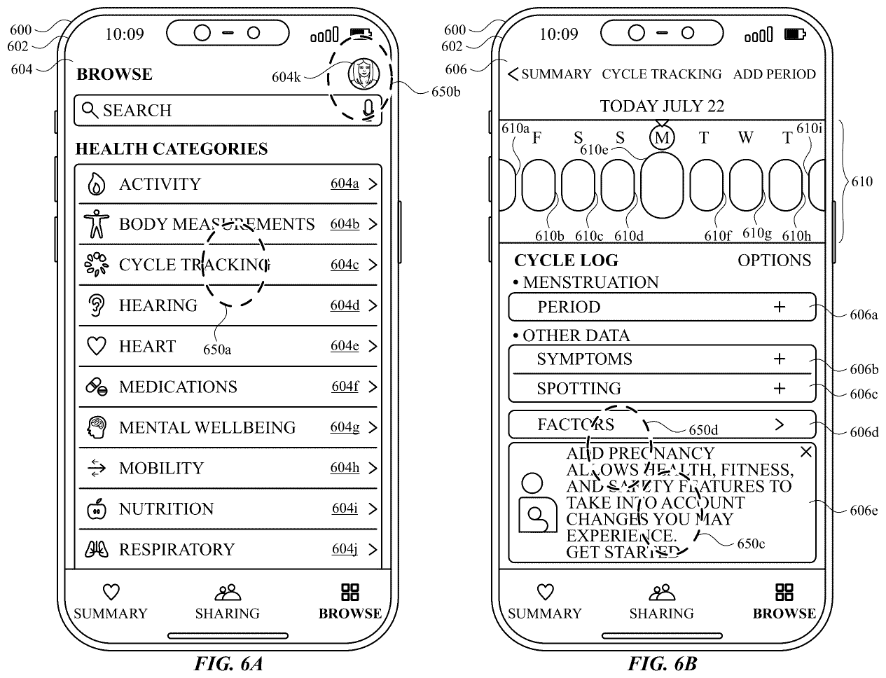

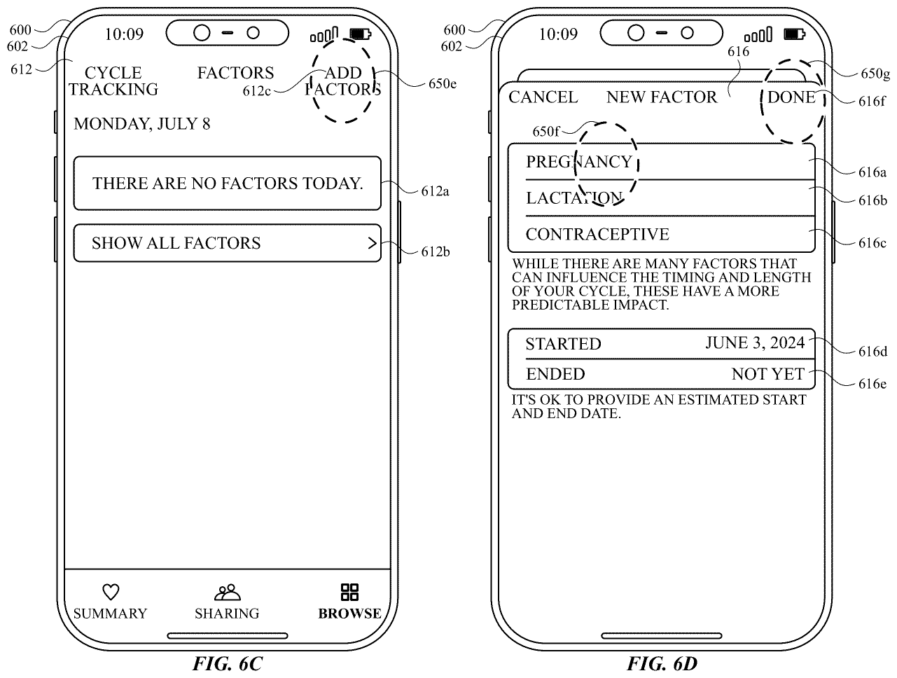

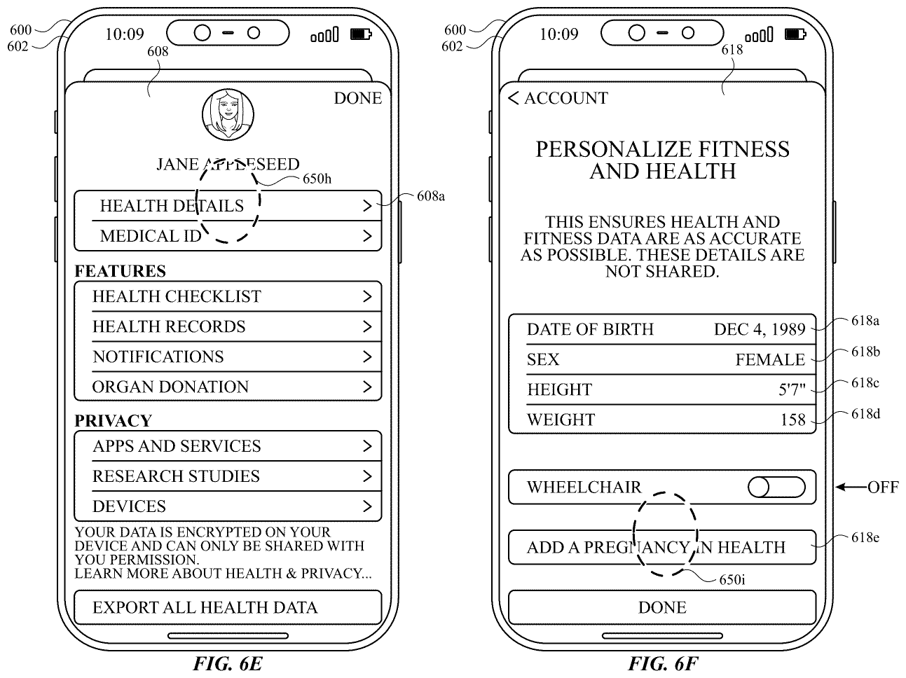

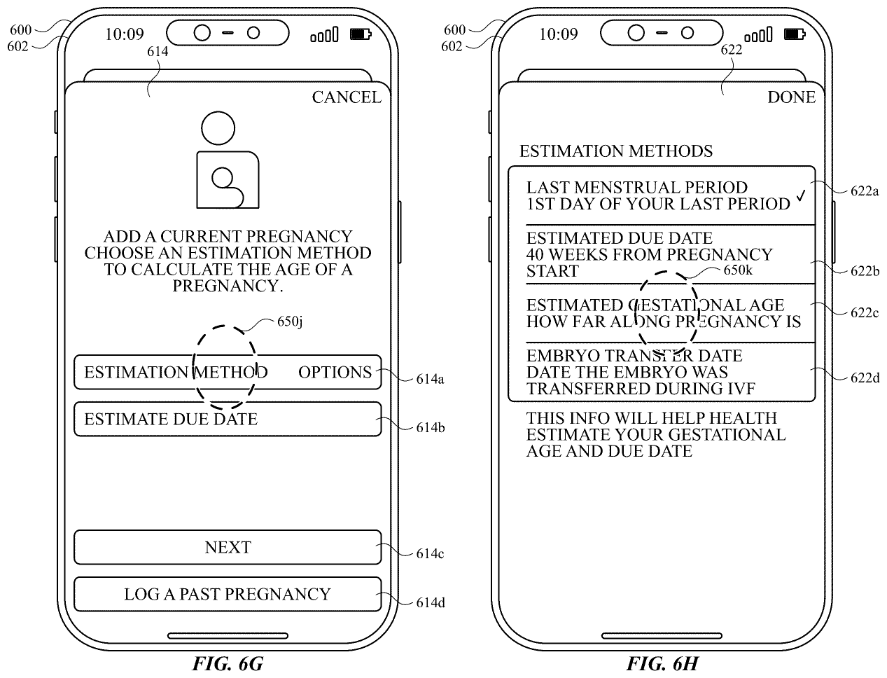

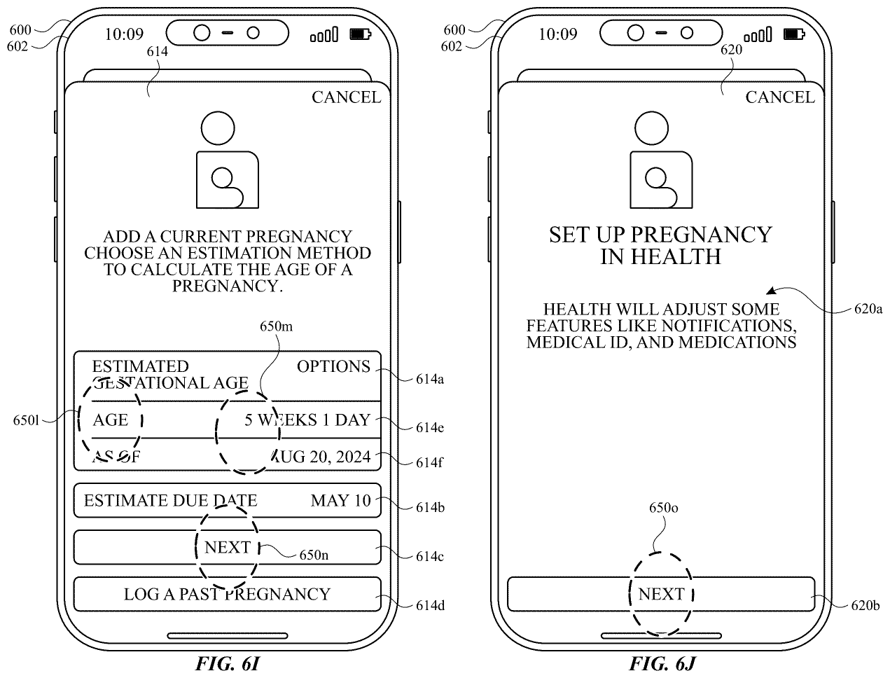

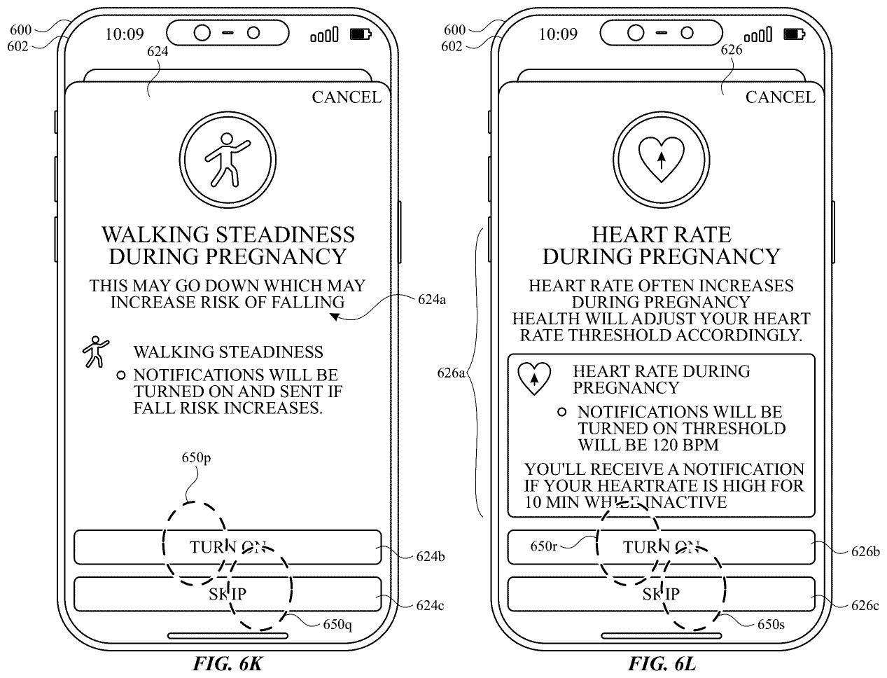

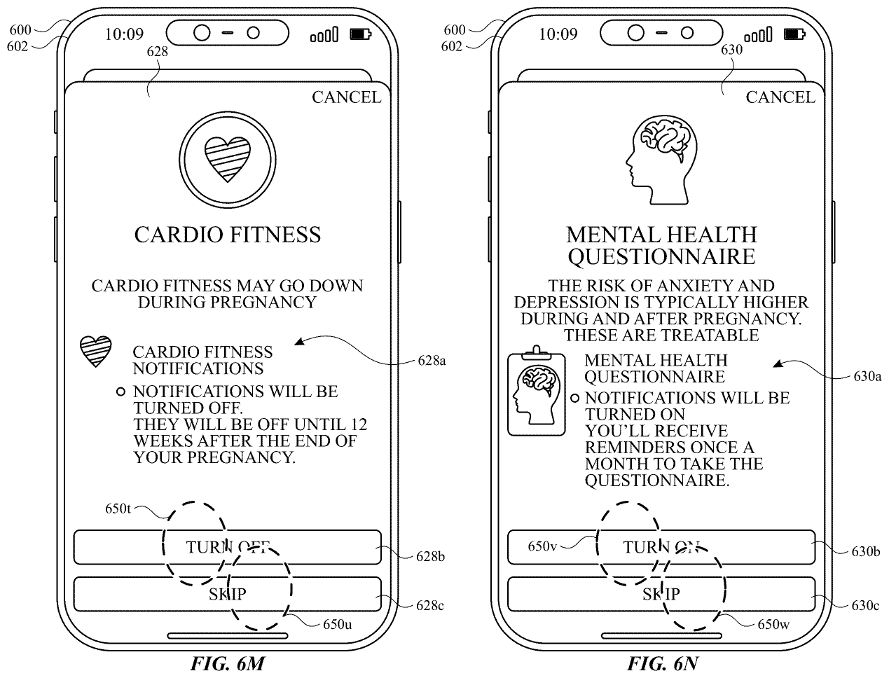

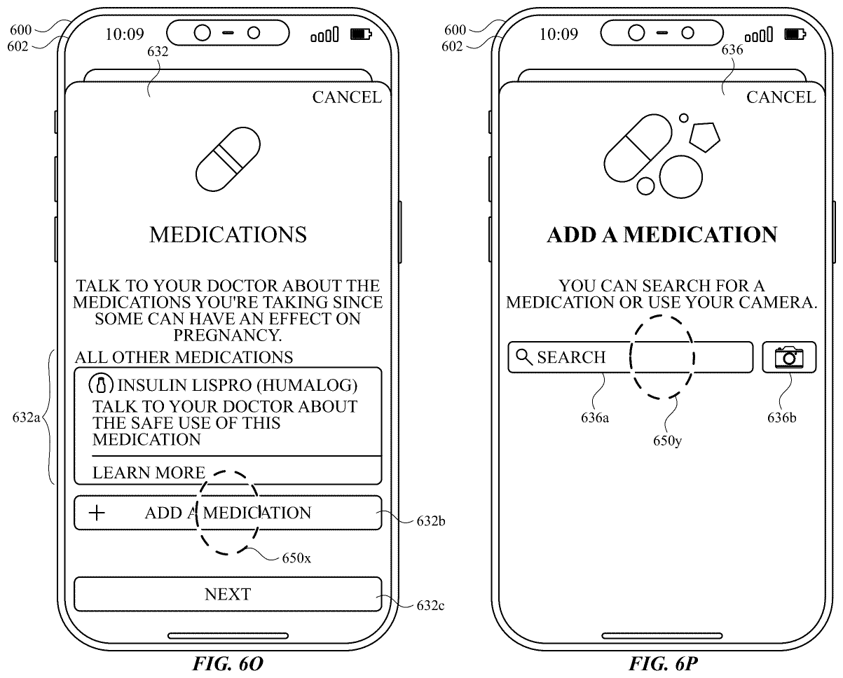

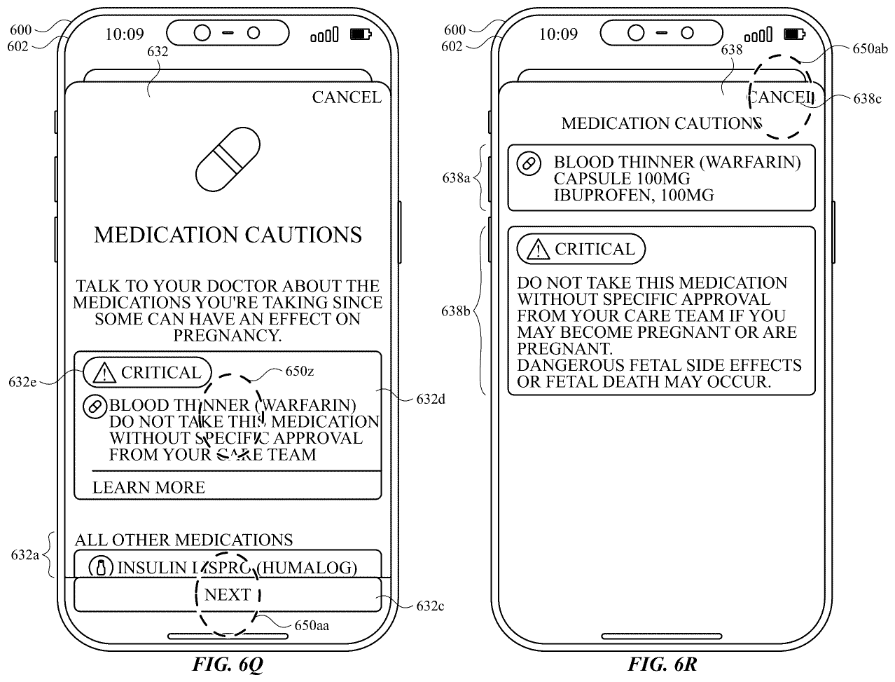

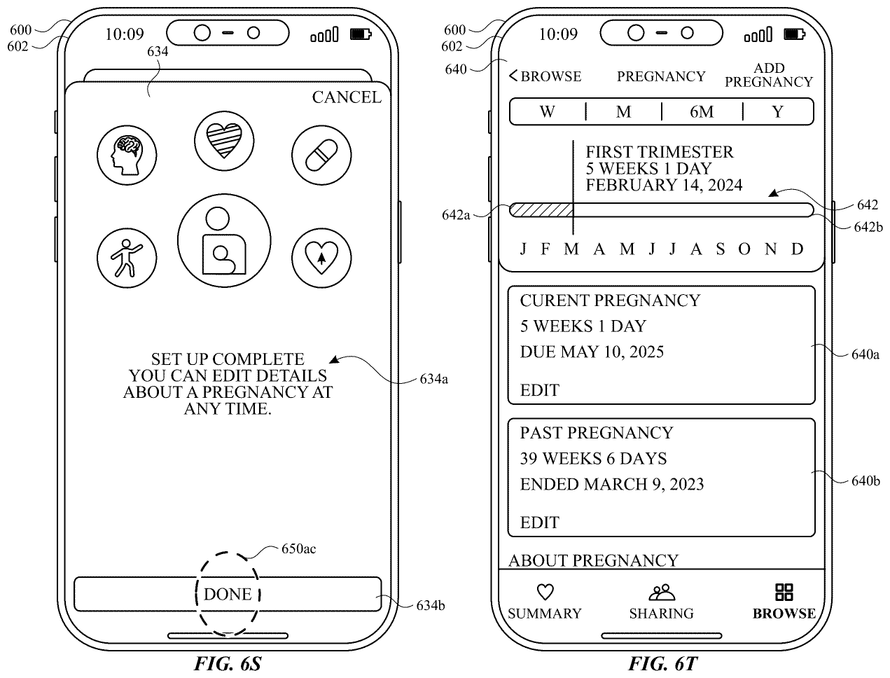

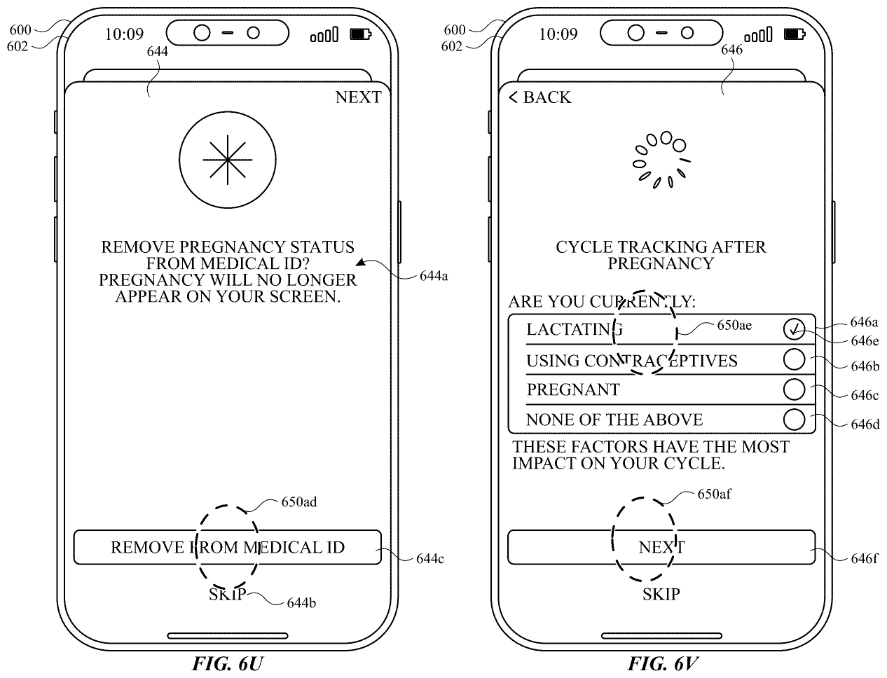

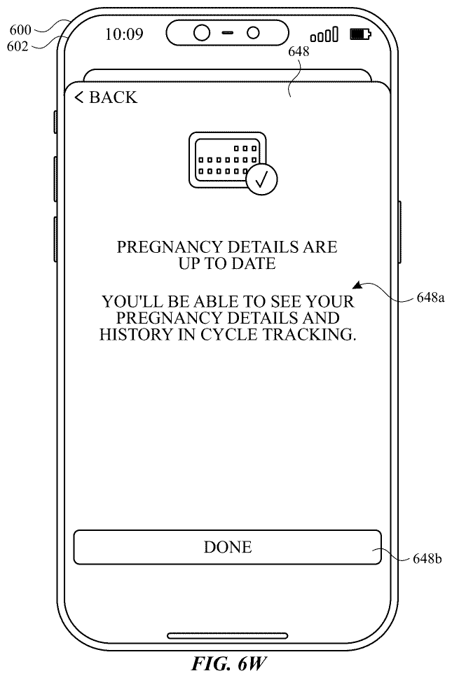

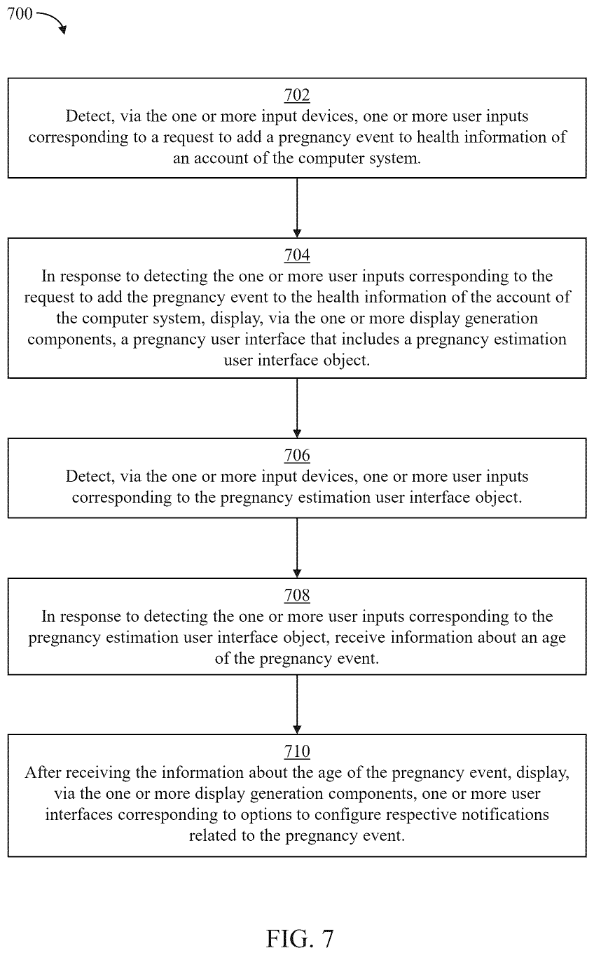

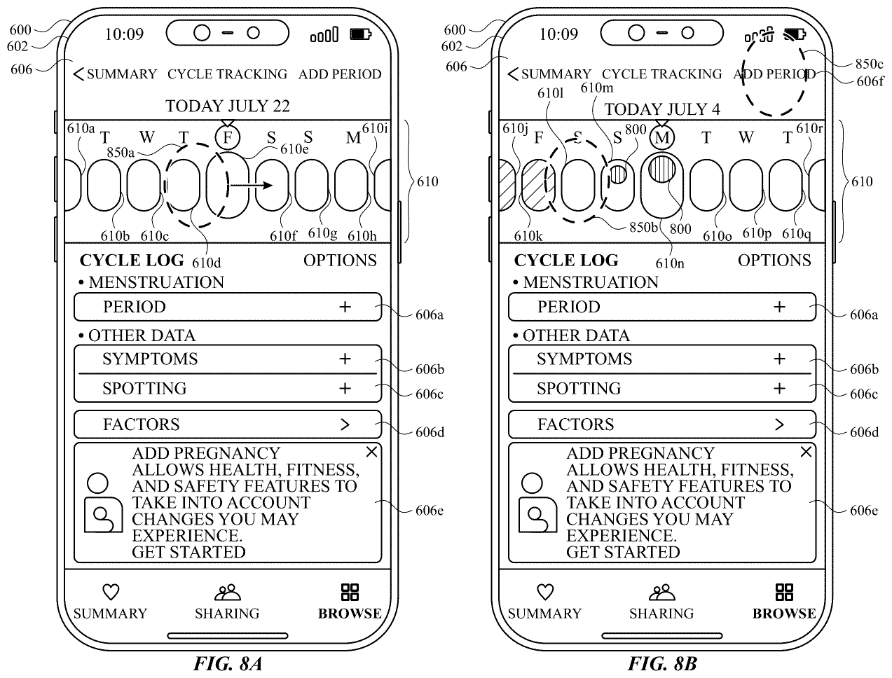

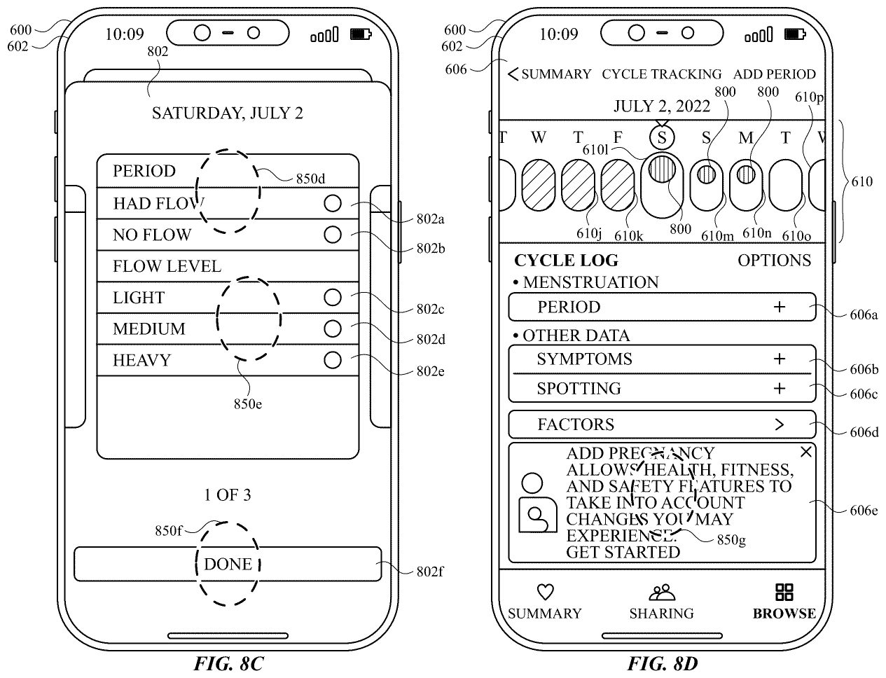

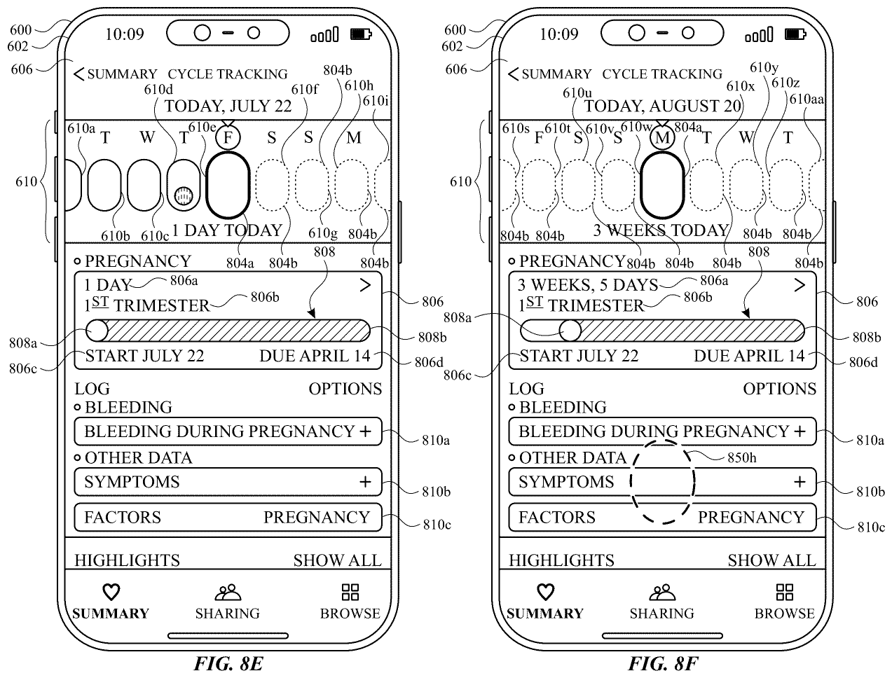

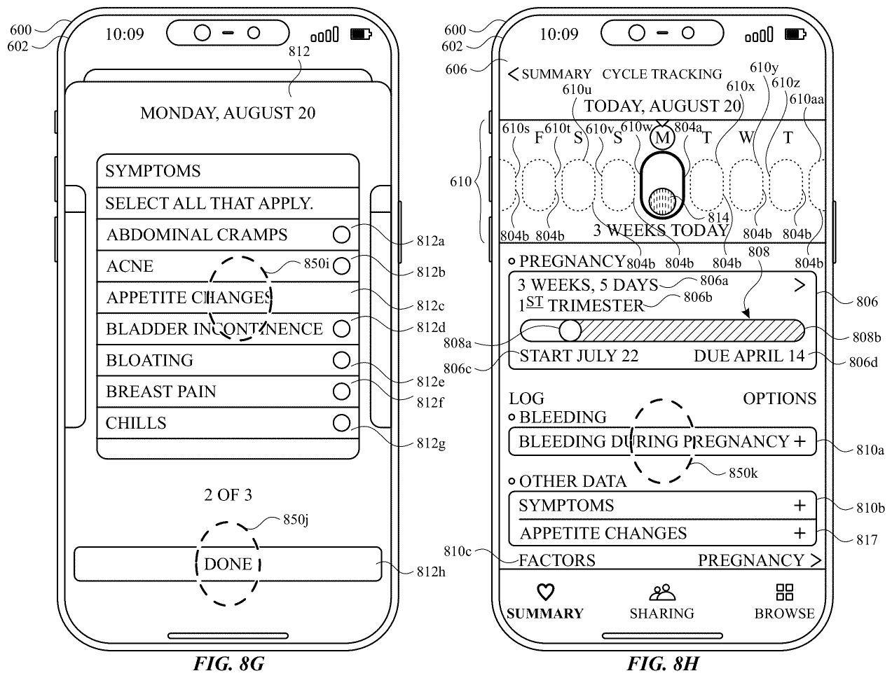

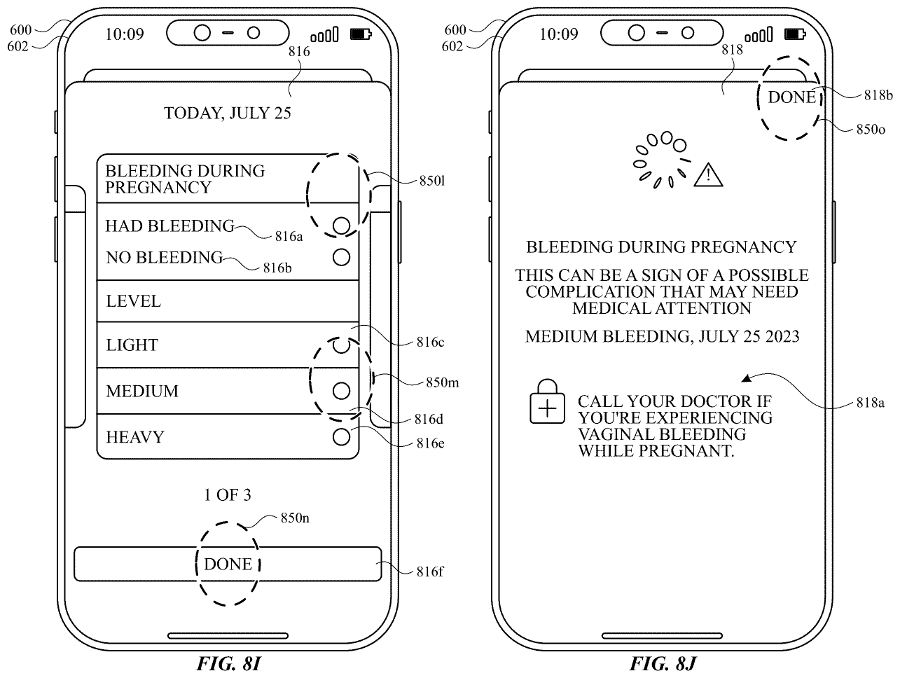

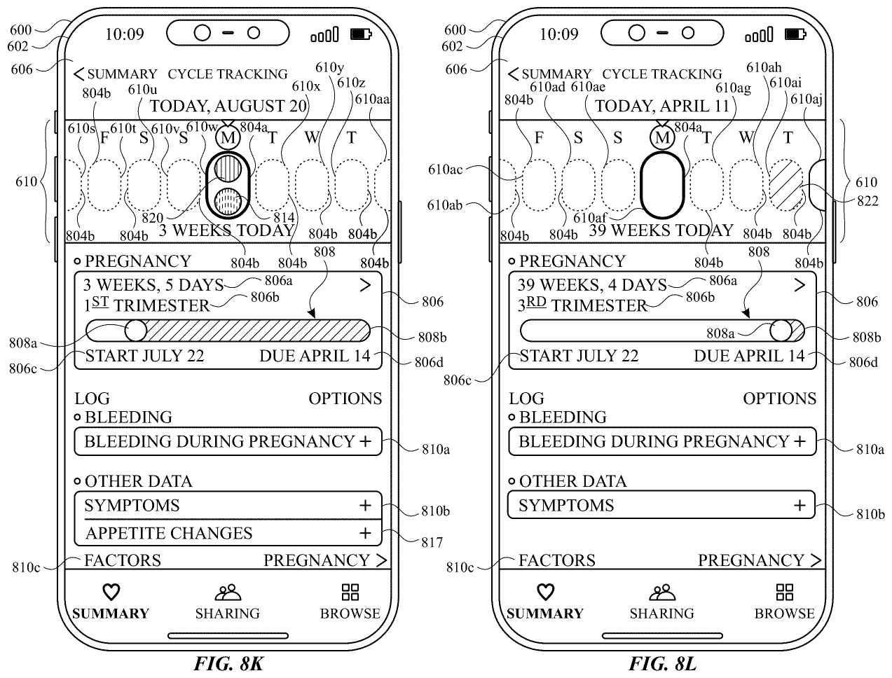

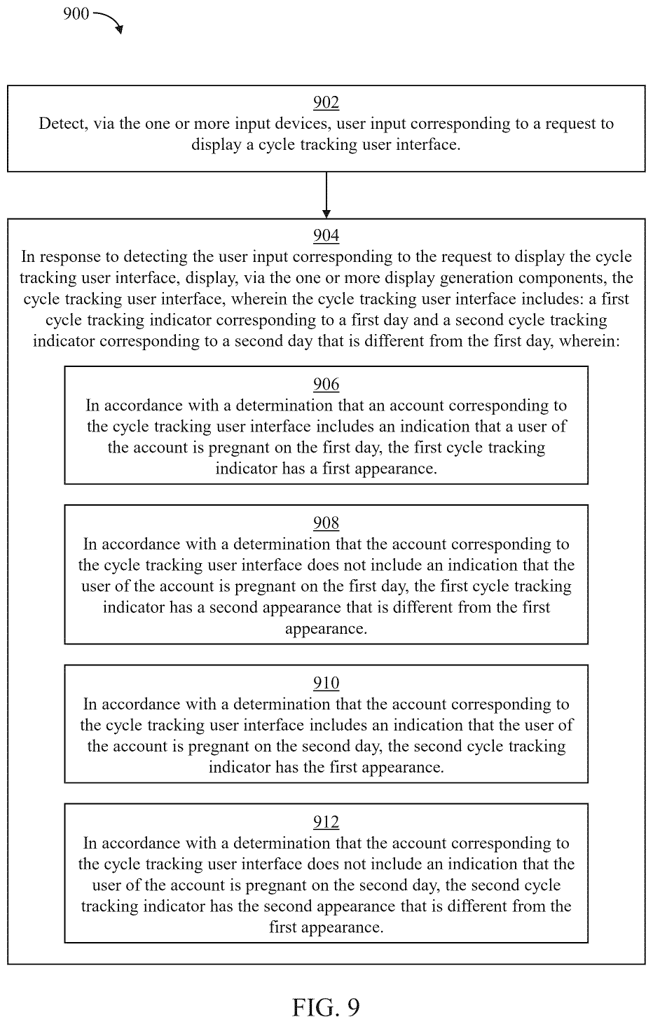

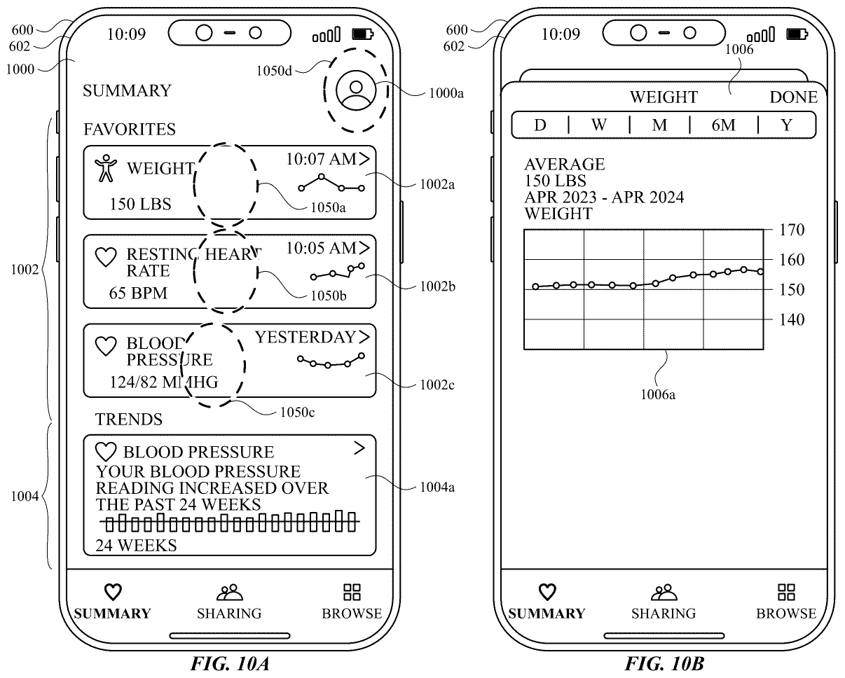

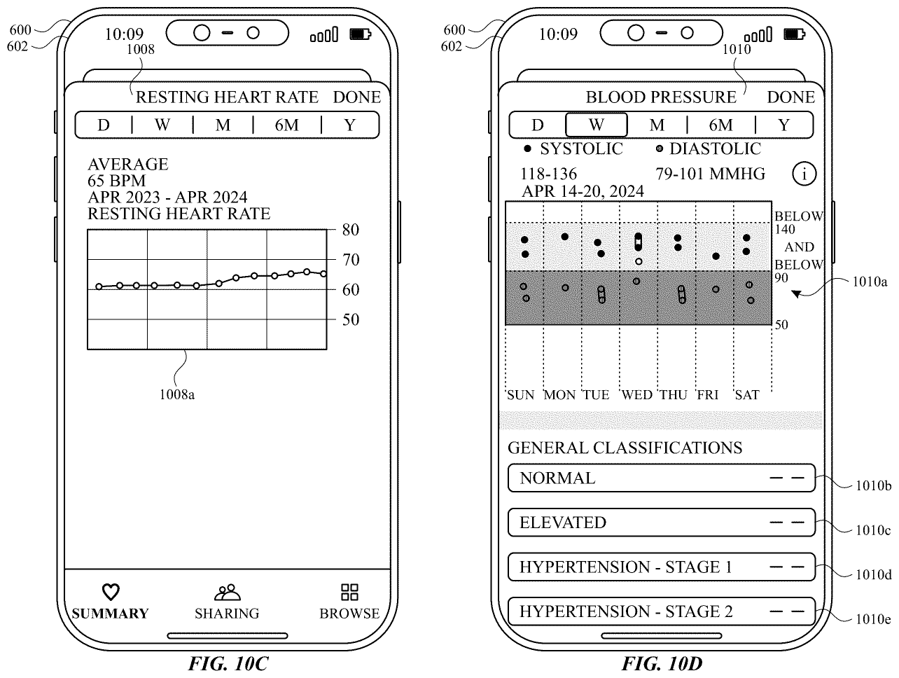

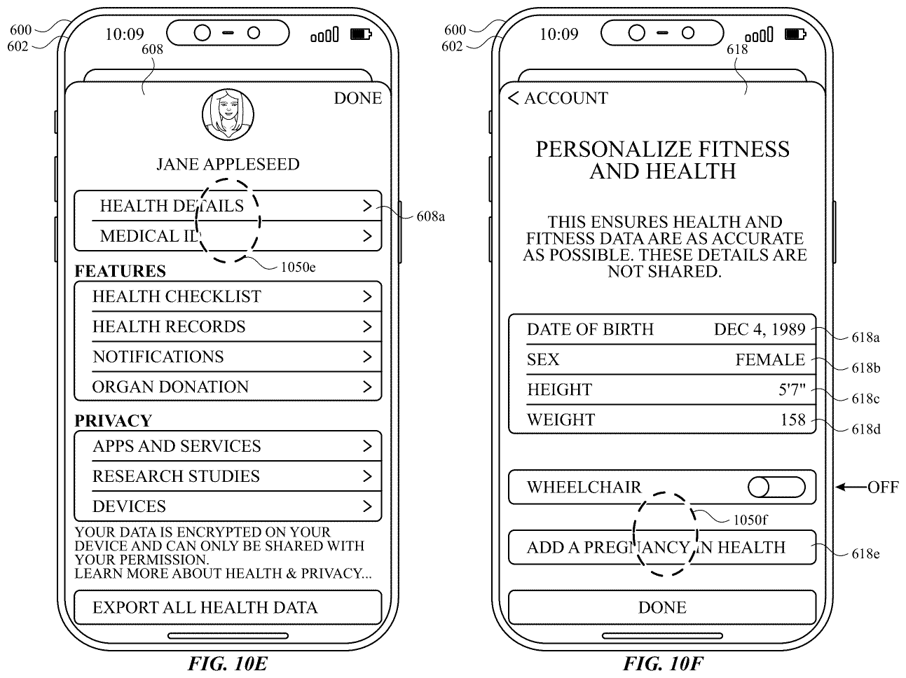

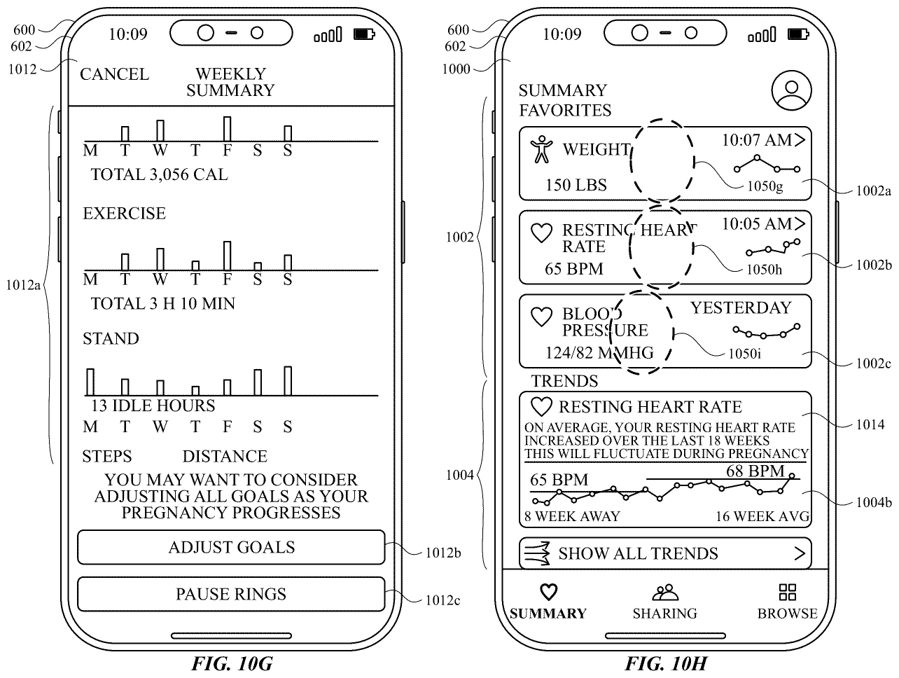

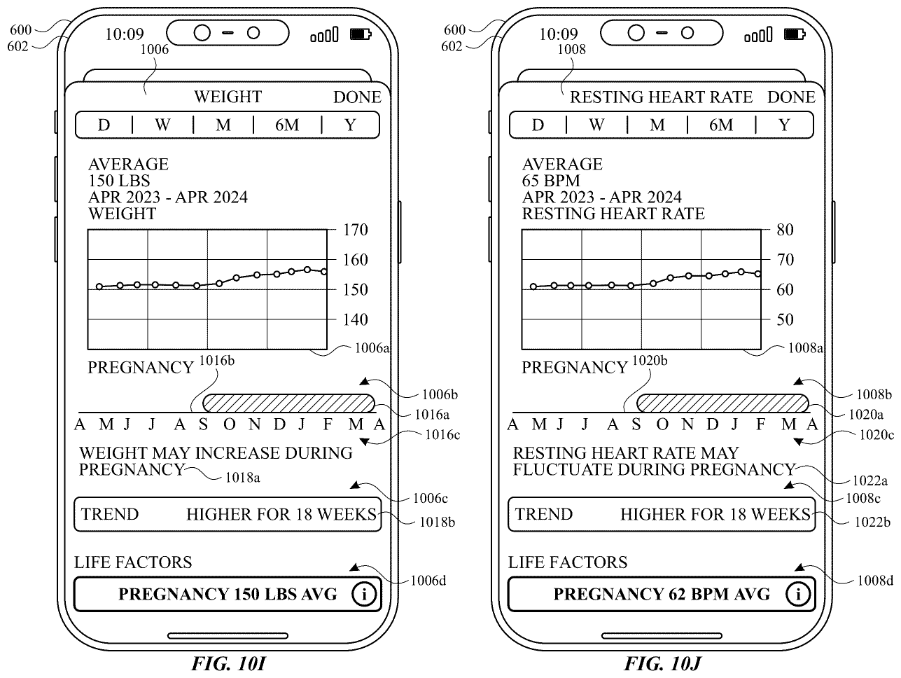

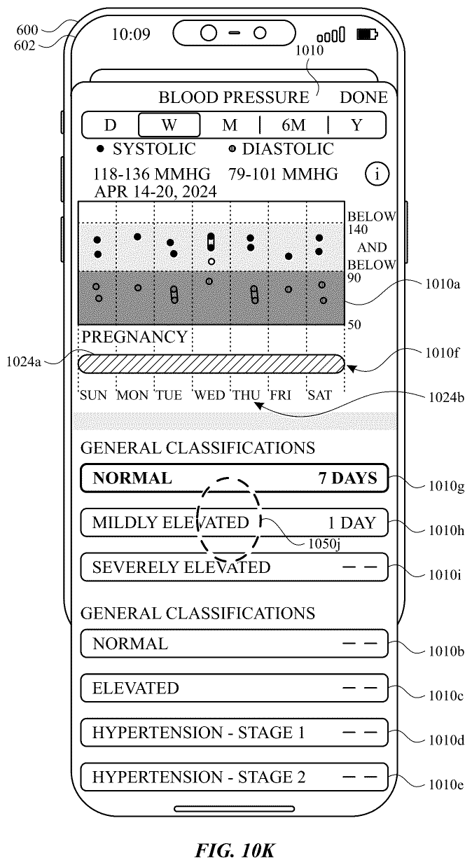

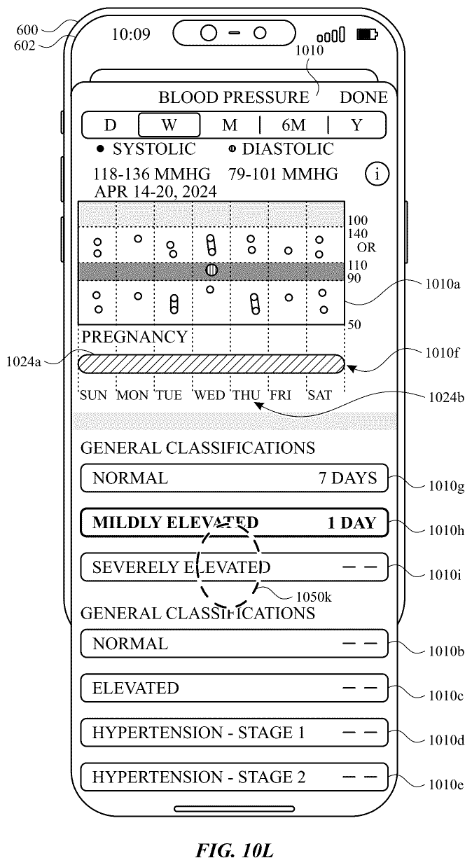

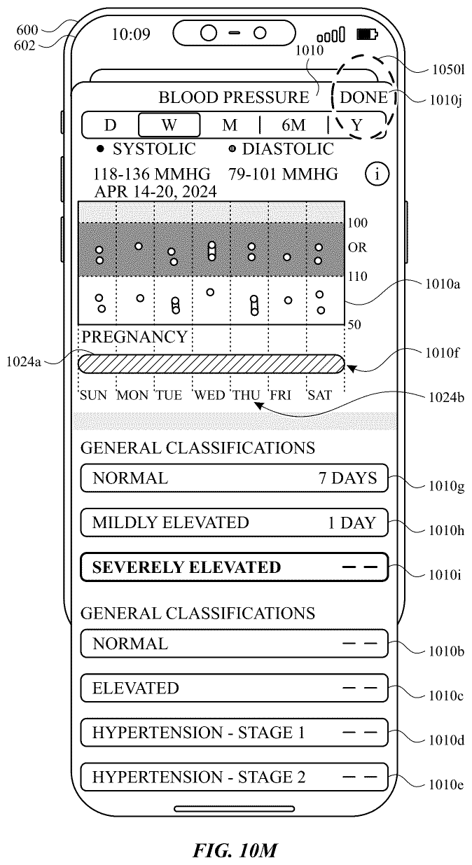

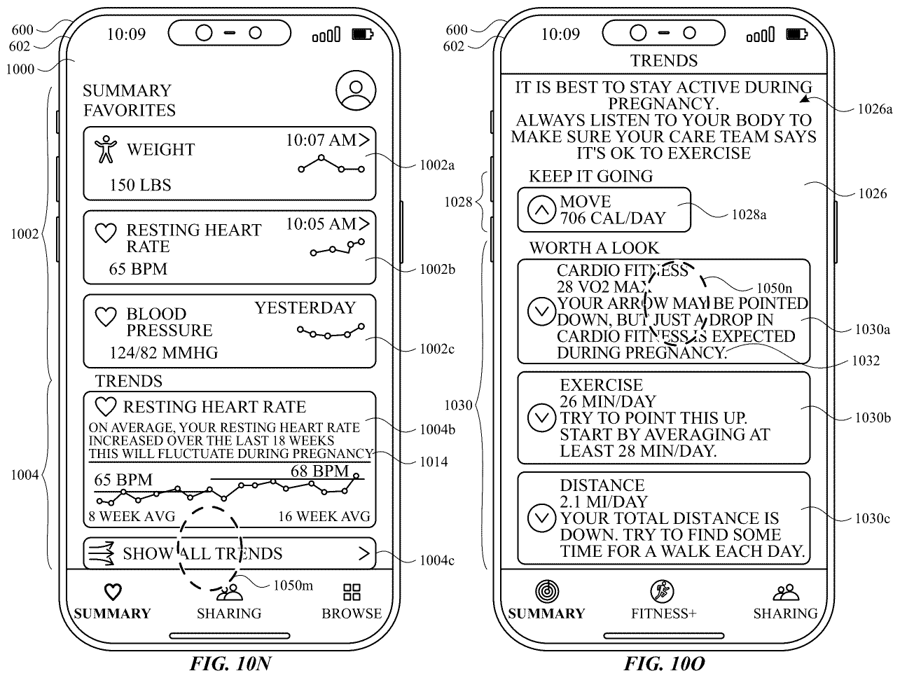

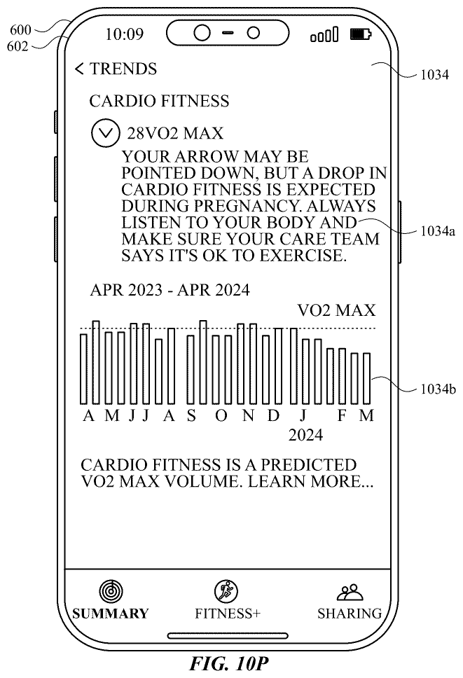

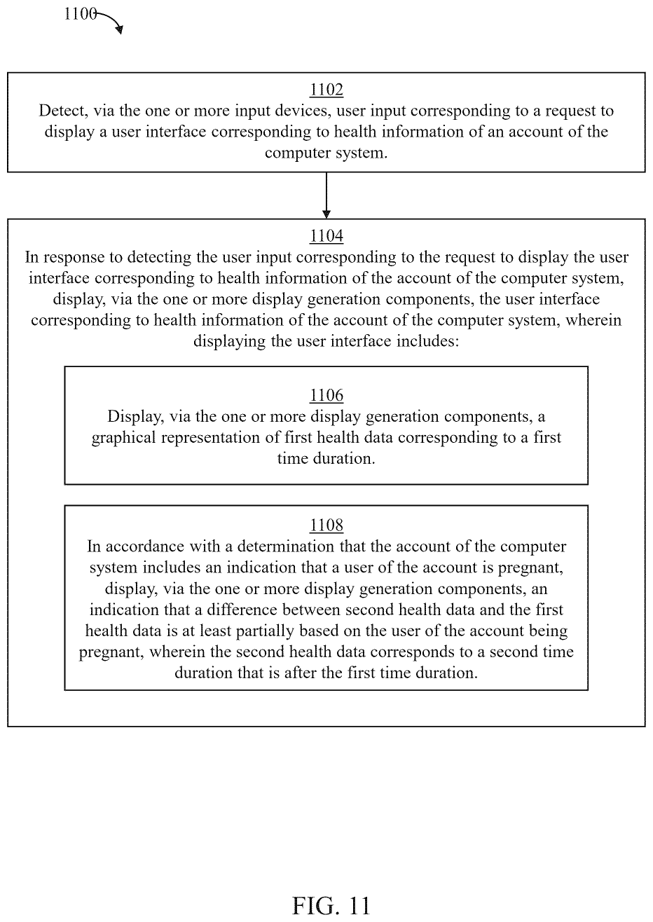

Below, FIGS. 1A-1B, 2, 3A-3G, 4A-4B, and 5A-5B provide a description of exemplary devices for performing the techniques for managing event notifications. FIGS. 6A-6W illustrate exemplary user interfaces for managing notifications related to a pregnancy event. FIG. 7 is a flow diagram illustrating methods of managing notifications related to a pregnancy event in accordance with some embodiments. The user interfaces in FIGS. 6A-6W are used to illustrate the processes described below, including the processes in FIG. 7. FIGS. 8A-8L illustrate exemplary user interfaces for tracking a pregnancy event. FIG. 9 is a flow diagram illustrating methods of tracking a pregnancy event in accordance with some embodiments. The user interfaces in FIGS. 8A-8L are used to illustrate the processes described below, including the processes in FIG. 9. FIGS. 10A-10P illustrate exemplary user interfaces for tracking health information during a pregnancy event. FIG. 11 is a flow diagram illustrating methods of tracking health information during a pregnancy event in accordance with some embodiments.

The processes described below enhance the operability of the devices and make the user-device interfaces more efficient (e.g., by helping the user to provide proper inputs and reducing user mistakes when operating/interacting with the device) through various techniques, including by providing improved visual feedback to the user, reducing the number of inputs needed to perform an operation, providing additional control options without cluttering the user interface with additional displayed controls, performing an operation when a set of conditions has been met without requiring further user input, and/or additional techniques. These techniques also reduce power usage and improve battery life of the device by enabling the user to use the device more quickly and efficiently.

In addition, in methods described herein where one or more steps are contingent upon one or more conditions having been met, it should be understood that the described method can be repeated in multiple repetitions so that over the course of the repetitions all of the conditions upon which steps in the method are contingent have been met in different repetitions of the method. For example, if a method requires performing a first step if a condition is satisfied, and a second step if the condition is not satisfied, then a person of ordinary skill would appreciate that the claimed steps are repeated until the condition has been both satisfied and not satisfied, in no particular order. Thus, a method described with one or more steps that are contingent upon one or more conditions having been met could be rewritten as a method that is repeated until each of the conditions described in the method has been met. This, however, is not required of system or computer readable medium claims where the system or computer readable medium contains instructions for performing the contingent operations based on the satisfaction of the corresponding one or more conditions and thus is capable of determining whether the contingency has or has not been satisfied without explicitly repeating steps of a method until all of the conditions upon which steps in the method are contingent have been met. A person having ordinary skill in the art would also understand that, similar to a method with contingent steps, a system or computer readable storage medium can repeat the steps of a method as many times as are needed to ensure that all of the contingent steps have been performed.

Although the following description uses terms “first,” “second,” etc. to describe various elements, these elements should not be limited by the terms. In some embodiments, these terms are used to distinguish one element from another. For example, a first touch could be termed a second touch, and, similarly, a second touch could be termed a first touch, without departing from the scope of the various described embodiments. In some embodiments, the first touch and the second touch are two separate references to the same touch. In some embodiments, the first touch and the second touch are both touches, but they are not the same touch.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

Embodiments of electronic devices, user interfaces for such devices, and associated processes for using such devices are described. In some embodiments, the device is a portable communications device, such as a mobile telephone, that also contains other functions, such as PDA and/or music player functions. Exemplary embodiments of portable multifunction devices include, without limitation, the iPhone®, iPod Touch®, and iPad® devices from Apple Inc. of Cupertino, California. Other portable electronic devices, such as laptops or tablet computers with touch-sensitive surfaces (e.g., touch screen displays and/or touchpads), are, optionally, used. It should also be understood that, in some embodiments, the device is not a portable communications device, but is a desktop computer with a touch-sensitive surface (e.g., a touch screen display and/or a touchpad). In some embodiments, the electronic device is a computer system that is in communication (e.g., via wireless communication, via wired communication) with a display generation component (e.g., a display device such as a head-mounted display (HM D), a display, a projector, a touch-sensitive display, or other device or component that presents visual content to a user, for example on or in the display generation component itself or produced from the display generation component and visible elsewhere). The display generation component is configured to provide visual output, such as display via a CRT display, display via an LED display, or display via image projection. In some embodiments, the display generation component is integrated with the computer system. In some embodiments, the display generation component is separate from the computer system. As used herein, “displaying” content includes causing to display the content (e.g., video data rendered or decoded by display controller 156) by transmitting, via a wired or wireless connection, data (e.g., image data or video data) to an integrated or external display generation component to visually produce the content.

In the discussion that follows, an electronic device that includes a display and a touch-sensitive surface is described. It should be understood, however, that the electronic device optionally includes one or more other physical user-interface devices, such as a physical keyboard, a mouse, and/or a joystick.

The device typically supports a variety of applications, such as one or more of the following: a drawing application, a presentation application, a word processing application, a website creation application, a disk authoring application, a spreadsheet application, a gaming application, a telephone application, a video conferencing application, an e-mail application, an instant messaging application, a workout support application, a photo management application, a digital camera application, a digital video camera application, a web browsing application, a digital music player application, and/or a digital video player application.

The various applications that are executed on the device optionally use at least one common physical user-interface device, such as the touch-sensitive surface. One or more functions of the touch-sensitive surface as well as corresponding information displayed on the device are, optionally, adjusted and/or varied from one application to the next and/or within a respective application. In this way, a common physical architecture (such as the touch-sensitive surface) of the device optionally supports the variety of applications with user interfaces that are intuitive and transparent to the user.

Attention is now directed toward embodiments of portable devices with touch-sensitive displays. FIG. 1A is a block diagram illustrating portable multifunction device 100 with touch-sensitive display system 112 in accordance with some embodiments. Touch-sensitive display 112 is sometimes called a “touch screen” for convenience and is sometimes known as or called a “touch-sensitive display system.” Device 100 includes memory 102 (which optionally includes one or more computer-readable storage media), memory controller 122, one or more processing units (CPUs) 120, peripherals interface 118, RF circuitry 108, audio circuitry 110, speaker 111, microphone 113, input/output (I/O) subsystem 106, other input control devices 116, and external port 124. Device 100 optionally includes one or more optical sensors 164. Device 100 optionally includes one or more contact intensity sensors 165 for detecting intensity of contacts on device 100 (e.g., a touch-sensitive surface such as touch-sensitive display system 112 of device 100). Device 100 optionally includes one or more tactile output generators 167 for generating tactile outputs on device 100 (e.g., generating tactile outputs on a touch-sensitive surface such as touch-sensitive display system 112 of device 100 or touchpad 355 of device 300). These components optionally communicate over one or more communication buses or signal lines 103.

As used in the specification and claims, the term “intensity” of a contact on a touch-sensitive surface refers to the force or pressure (force per unit area) of a contact (e.g., a finger contact) on the touch-sensitive surface, or to a substitute (proxy) for the force or pressure of a contact on the touch-sensitive surface. The intensity of a contact has a range of values that includes at least four distinct values and more typically includes hundreds of distinct values (e.g., at least 256). Intensity of a contact is, optionally, determined (or measured) using various approaches and various sensors or combinations of sensors. For example, one or more force sensors underneath or adjacent to the touch-sensitive surface are, optionally, used to measure force at various points on the touch-sensitive surface. In some implementations, force measurements from multiple force sensors are combined (e.g., a weighted average) to determine an estimated force of a contact. Similarly, a pressure-sensitive tip of a stylus is, optionally, used to determine a pressure of the stylus on the touch-sensitive surface. Alternatively, the size of the contact area detected on the touch-sensitive surface and/or changes thereto, the capacitance of the touch-sensitive surface proximate to the contact and/or changes thereto, and/or the resistance of the touch-sensitive surface proximate to the contact and/or changes thereto are, optionally, used as a substitute for the force or pressure of the contact on the touch-sensitive surface. In some implementations, the substitute measurements for contact force or pressure are used directly to determine whether an intensity threshold has been exceeded (e.g., the intensity threshold is described in units corresponding to the substitute measurements). In some implementations, the substitute measurements for contact force or pressure are converted to an estimated force or pressure, and the estimated force or pressure is used to determine whether an intensity threshold has been exceeded (e.g., the intensity threshold is a pressure threshold measured in units of pressure). Using the intensity of a contact as an attribute of a user input allows for user access to additional device functionality that may otherwise not be accessible by the user on a reduced-size device with limited real estate for displaying affordances (e.g., on a touch-sensitive display) and/or receiving user input (e.g., via a touch-sensitive display, a touch-sensitive surface, or a physical/mechanical control such as a knob or a button).

As used in the specification and claims, the term “tactile output” refers to physical displacement of a device relative to a previous position of the device, physical displacement of a component (e.g., a touch-sensitive surface) of a device relative to another component (e.g., housing) of the device, or displacement of the component relative to a center of mass of the device that will be detected by a user with the user's sense of touch. For example, in situations where the device or the component of the device is in contact with a surface of a user that is sensitive to touch (e.g., a finger, palm, or other part of a user's hand), the tactile output generated by the physical displacement will be interpreted by the user as a tactile sensation corresponding to a perceived change in physical characteristics of the device or the component of the device. For example, movement of a touch-sensitive surface (e.g., a touch-sensitive display or trackpad) is, optionally, interpreted by the user as a “down click” or “up click” of a physical actuator button. In some cases, a user will feel a tactile sensation such as an “down click” or “up click” even when there is no movement of a physical actuator button associated with the touch-sensitive surface that is physically pressed (e.g., displaced) by the user's movements. As another example, movement of the touch-sensitive surface is, optionally, interpreted or sensed by the user as “roughness” of the touch-sensitive surface, even when there is no change in smoothness of the touch-sensitive surface. While such interpretations of touch by a user will be subject to the individualized sensory perceptions of the user, there are many sensory perceptions of touch that are common to a large majority of users. Thus, when a tactile output is described as corresponding to a particular sensory perception of a user (e.g., an “up click,” a “down click,” “roughness”), unless otherwise stated, the generated tactile output corresponds to physical displacement of the device or a component thereof that will generate the described sensory perception for a typical (or average) user.

It should be appreciated that device 100 is only one example of a portable multifunction device, and that device 100 optionally has more or fewer components than shown, optionally combines two or more components, or optionally has a different configuration or arrangement of the components. The various components shown in FIG. 1A are implemented in hardware, software, or a combination of both hardware and software, including one or more signal processing and/or application-specific integrated circuits.

Memory 102 optionally includes high-speed random access memory and optionally also includes non-volatile memory, such as one or more magnetic disk storage devices, flash memory devices, or other non-volatile solid-state memory devices. Memory controller 122 optionally controls access to memory 102 by other components of device 100.

Peripherals interface 118 can be used to couple input and output peripherals of the device to CPU 120 and memory 102. The one or more processors 120 run or execute various software programs (such as computer programs (e.g., including instructions)) and/or sets of instructions stored in memory 102 to perform various functions for device 100 and to process data. In some embodiments, peripherals interface 118, CPU 120, and memory controller 122 are, optionally, implemented on a single chip, such as chip 104. In some other embodiments, they are, optionally, implemented on separate chips.

RF (radio frequency) circuitry 108 receives and sends RF signals, also called electromagnetic signals. RF circuitry 108 converts electrical signals to/from electromagnetic signals and communicates with communications networks and other communications devices via the electromagnetic signals. RF circuitry 108 optionally includes well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth. RF circuitry 108 optionally communicates with networks, such as the Internet, also referred to as the World Wide Web (WWW), an intranet and/or a wireless network, such as a cellular telephone network, a wireless local area network (LAN) and/or a metropolitan area network (MAN), and other devices by wireless communication. The RF circuitry 108 optionally includes well-known circuitry for detecting near field communication (NFC) fields, such as by a short-range communication radio. The wireless communication optionally uses any of a plurality of communications standards, protocols, and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDM A), Bluetooth, Bluetooth Low Energy (BTLE), Wireless Fidelity (Wi-Fi) (e.g., IEEE 802.11a, IEEE 802.11b, IEEE 802.11g, IEEE 802.11n, and/or IEEE 802.11ac), voice over Internet Protocol (VoIP), Wi-MAX, a protocol for e-mail (e.g., Internet message access protocol (IM A P) and/or post office protocol (POP)), instant messaging (e.g., extensible messaging and presence protocol (XM PP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

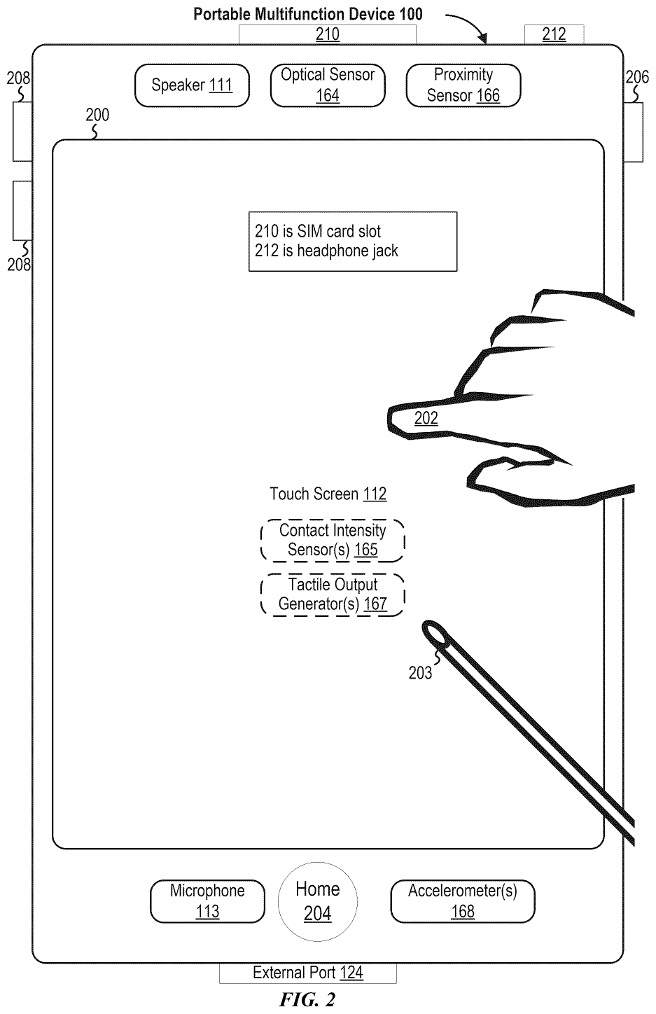

Audio circuitry 110, speaker 111, and microphone 113 provide an audio interface between a user and device 100. Audio circuitry 110 receives audio data from peripherals interface 118, converts the audio data to an electrical signal, and transmits the electrical signal to speaker 111. Speaker 111 converts the electrical signal to human-audible sound waves. Audio circuitry 110 also receives electrical signals converted by microphone 113 from sound waves. Audio circuitry 110 converts the electrical signal to audio data and transmits the audio data to peripherals interface 118 for processing. Audio data is, optionally, retrieved from and/or transmitted to memory 102 and/or RF circuitry 108 by peripherals interface 118. In some embodiments, audio circuitry 110 also includes a headset jack (e.g., 212, FIG. 2). The headset jack provides an interface between audio circuitry 110 and removable audio input/output peripherals, such as output-only headphones or a headset with both output (e.g., a headphone for one or both ears) and input (e.g., a microphone).

I/O subsystem 106 couples input/output peripherals on device 100, such as touch screen 112 and other input control devices 116, to peripherals interface 118. I/O subsystem 106 optionally includes display controller 156, optical sensor controller 158, depth camera controller 169, intensity sensor controller 159, haptic feedback controller 161, and one or more input controllers 160 for other input or control devices. The one or more input controllers 160 receive/send electrical signals from/to other input control devices 116. The other input control devices 116 optionally include physical buttons (e.g., push buttons, rocker buttons, etc.), dials, slider switches, joysticks, click wheels, and so forth. In some embodiments, input controller(s) 160 are, optionally, coupled to any (or none) of the following: a keyboard, an infrared port, a USB port, and a pointer device such as a mouse. The one or more buttons (e.g., 208, FIG. 2) optionally include an up/down button for volume control of speaker 111 and/or microphone 113. The one or more buttons optionally include a push button (e.g., 206, FIG. 2). In some embodiments, the electronic device is a computer system that is in communication (e.g., via wireless communication, via wired communication) with one or more input devices. In some embodiments, the one or more input devices include a touch-sensitive surface (e.g., a trackpad, as part of a touch-sensitive display). In some embodiments, the one or more input devices include one or more camera sensors (e.g., one or more optical sensors 164 and/or one or more depth camera sensors 175), such as for tracking a user's gestures (e.g., hand gestures and/or air gestures) as input. In some embodiments, the one or more input devices are integrated with the computer system. In some embodiments, the one or more input devices are separate from the computer system. In some embodiments, an air gesture is a gesture that is detected without the user touching an input element that is part of the device (or independently of an input element that is a part of the device) and is based on detected motion of a portion of the user's body through the air including motion of the user's body relative to an absolute reference (e.g., an angle of the user's arm relative to the ground or a distance of the user's hand relative to the ground), relative to another portion of the user's body (e.g., movement of a hand of the user relative to a shoulder of the user, movement of one hand of the user relative to another hand of the user, and/or movement of a finger of the user relative to another finger or portion of a hand of the user), and/or absolute motion of a portion of the user's body (e.g., a tap gesture that includes movement of a hand in a predetermined pose by a predetermined amount and/or speed, or a shake gesture that includes a predetermined speed or amount of rotation of a portion of the user's body).

A quick press of the push button optionally disengages a lock of touch screen 112 or optionally begins a process that uses gestures on the touch screen to unlock the device, as described in U.S. patent application Ser. No. 11/322,549, “Unlocking a Device by Performing Gestures on an Unlock Image,” filed Dec. 23, 2005, U.S. Pat. No. 7,657,849, which is hereby incorporated by reference in its entirety. A longer press of the push button (e.g., 206) optionally turns power to device 100 on or off. The functionality of one or more of the buttons are, optionally, user-customizable. Touch screen 112 is used to implement virtual or soft buttons and one or more soft keyboards.

Touch-sensitive display 112 provides an input interface and an output interface between the device and a user. Display controller 156 receives and/or sends electrical signals from/to touch screen 112. Touch screen 112 displays visual output to the user. The visual output optionally includes graphics, text, icons, video, and any combination thereof (collectively termed “graphics”). In some embodiments, some or all of the visual output optionally corresponds to user-interface objects.

Touch screen 112 has a touch-sensitive surface, sensor, or set of sensors that accepts input from the user based on haptic and/or tactile contact. Touch screen 112 and display controller 156 (along with any associated modules and/or sets of instructions in memory 102) detect contact (and any movement or breaking of the contact) on touch screen 112 and convert the detected contact into interaction with user-interface objects (e.g., one or more soft keys, icons, web pages, or images) that are displayed on touch screen 112. In an exemplary embodiment, a point of contact between touch screen 112 and the user corresponds to a finger of the user.

Touch screen 112 optionally uses LCD (liquid crystal display) technology, LPD (light emitting polymer display) technology, or LED (light emitting diode) technology, although other display technologies are used in other embodiments. Touch screen 112 and display controller 156 optionally detect contact and any movement or breaking thereof using any of a plurality of touch sensing technologies now known or later developed, including but not limited to capacitive, resistive, infrared, and surface acoustic wave technologies, as well as other proximity sensor arrays or other elements for determining one or more points of contact with touch screen 112. In an exemplary embodiment, projected mutual capacitance sensing technology is used, such as that found in the iPhone® and iPod Touch® from Apple Inc. of Cupertino, California.

A touch-sensitive display in some embodiments of touch screen 112 is, optionally, analogous to the multi-touch sensitive touchpads described in the following U.S. Pat. No. 6,323,846 (Westerman et al.), 6,570,557 (Westerman et al.), and/or 6,677,932 (Westerman), and/or U.S. Patent Publication 2002/0015024A1, each of which is hereby incorporated by reference in its entirety. However, touch screen 112 displays visual output from device 100, whereas touch-sensitive touchpads do not provide visual output.

A touch-sensitive display in some embodiments of touch screen 112 is described in the following applications: (1) U.S. patent application Ser. No. 11/381,313, “Multipoint Touch Surface Controller,” filed May 2, 2006; (2) U.S. patent application Ser. No. 10/840,862, “Multipoint Touchscreen,” filed May 6, 2004; (3) U.S. patent application Ser. No. 10/903,964, “Gestures For Touch Sensitive Input Devices,” filed Jul. 30, 2004; (4) U.S. patent application Ser. No. 11/048,264, “Gestures For Touch Sensitive Input Devices,” filed Jan. 31, 2005; (5) U.S. patent application Ser. No. 11/038,590, “Mode-Based Graphical User Interfaces For Touch Sensitive Input Devices,” filed Jan. 18, 2005; (6) U.S. patent application Ser. No. 11/228,758, “Virtual Input Device Placement On A Touch Screen User Interface,” filed Sep. 16, 2005; (7) U.S. patent application Ser. No. 11/228,700, “Operation Of A Computer With A Touch Screen Interface,” filed Sep. 16, 2005; (8) U.S. patent application Ser. No. 11/228,737, “Activating Virtual Keys Of A Touch-Screen Virtual Keyboard,” filed Sep. 16, 2005; and (9) U.S. patent application Ser. No. 11/367,749, “Multi-Functional Hand-Held Device,” filed Mar. 3, 2006. All of these applications are incorporated by reference herein in their entirety.

Touch screen 112 optionally has a video resolution in excess of 100 dpi. In some embodiments, the touch screen has a video resolution of approximately 160 dpi. The user optionally makes contact with touch screen 112 using any suitable object or appendage, such as a stylus, a finger, and so forth. In some embodiments, the user interface is designed to work primarily with finger-based contacts and gestures, which can be less precise than stylus-based input due to the larger area of contact of a finger on the touch screen. In some embodiments, the device translates the rough finger-based input into a precise pointer/cursor position or command for performing the actions desired by the user.

In some embodiments, in addition to the touch screen, device 100 optionally includes a touchpad for activating or deactivating particular functions. In some embodiments, the touchpad is a touch-sensitive area of the device that, unlike the touch screen, does not display visual output. The touchpad is, optionally, a touch-sensitive surface that is separate from touch screen 112 or an extension of the touch-sensitive surface formed by the touch screen.

Device 100 also includes power system 162 for powering the various components. Power system 162 optionally includes a power management system, one or more power sources (e.g., battery, alternating current (AC)), a recharging system, a power failure detection circuit, a power converter or inverter, a power status indicator (e.g., a light-emitting diode (LED)) and any other components associated with the generation, management and distribution of power in portable devices.

Device 100 optionally also includes secure element 163 for securely storing information. In some embodiments, secure element 163 is a hardware component (e.g., a secure microcontroller chip) configured to securely store data or an algorithm. In some embodiments, secure element 163 provides (e.g., releases) secure information (e.g., payment information (e.g., an account number and/or a transaction-specific dynamic security code), identification information (e.g., credentials of a state-approved digital identification), and/or authentication information (e.g., data generated using a cryptography engine and/or by performing asymmetric cryptography operations)). In some embodiments, secure element 163 provides (or releases) the secure information in response to device 100 receiving authorization, such as a user authentication (e.g., fingerprint authentication; passcode authentication; detecting double-press of a hardware button when device 100 is in an unlocked state, and optionally, while device 100 has been continuously on a user's wrist since device 100 was unlocked by providing authentication credentials to device 100, where the continuous presence of device 100 on the user's wrist is determined by periodically checking that the device is in contact with the user's skin). For example, device 100 detects a fingerprint at a fingerprint sensor (e.g., a fingerprint sensor integrated into a button) of device 100. Device 100 determines whether the detected fingerprint is consistent with an enrolled fingerprint. In accordance with a determination that the fingerprint is consistent with the enrolled fingerprint, secure element 163 provides (e.g., releases) the secure information. In accordance with a determination that the fingerprint is not consistent with the enrolled fingerprint, secure element 163 forgoes providing (e.g., releasing) the secure information.

Device 100 optionally also includes one or more optical sensors 164. FIG. 1A shows an optical sensor coupled to optical sensor controller 158 in I/O subsystem 106. Optical sensor 164 optionally includes charge-coupled device (CCD) or complementary metal-oxide semiconductor (CM OS) phototransistors. Optical sensor 164 receives light from the environment, projected through one or more lenses, and converts the light to data representing an image. In conjunction with imaging module 143 (also called a camera module), optical sensor 164 optionally captures still images or video. In some embodiments, an optical sensor is located on the back of device 100, opposite touch screen display 112 on the front of the device so that the touch screen display is enabled for use as a viewfinder for still and/or video image acquisition. In some embodiments, an optical sensor is located on the front of the device so that the user's image is, optionally, obtained for video conferencing while the user views the other video conference participants on the touch screen display. In some embodiments, the position of optical sensor 164 can be changed by the user (e.g., by rotating the lens and the sensor in the device housing) so that a single optical sensor 164 is used along with the touch screen display for both video conferencing and still and/or video image acquisition.

Device 100 optionally also includes one or more depth camera sensors 175. FIG. 1A shows a depth camera sensor coupled to depth camera controller 169 in I/O subsystem 106. Depth camera sensor 175 receives data from the environment to create a three dimensional model of an object (e.g., a face) within a scene from a viewpoint (e.g., a depth camera sensor). In some embodiments, in conjunction with imaging module 143 (also called a camera module), depth camera sensor 175 is optionally used to determine a depth map of different portions of an image captured by the imaging module 143. In some embodiments, a depth camera sensor is located on the front of device 100 so that the user's image with depth information is, optionally, obtained for video conferencing while the user views the other video conference participants on the touch screen display and to capture selfies with depth map data. In some embodiments, the depth camera sensor 175 is located on the back of device, or on the back and the front of the device 100. In some embodiments, the position of depth camera sensor 175 can be changed by the user (e.g., by rotating the lens and the sensor in the device housing) so that a depth camera sensor 175 is used along with the touch screen display for both video conferencing and still and/or video image acquisition.

Device 100 optionally also includes one or more contact intensity sensors 165. FIG. 1A shows a contact intensity sensor coupled to intensity sensor controller 159 in I/O subsystem 106. Contact intensity sensor 165 optionally includes one or more piezoresistive strain gauges, capacitive force sensors, electric force sensors, piezoelectric force sensors, optical force sensors, capacitive touch-sensitive surfaces, or other intensity sensors (e.g., sensors used to measure the force (or pressure) of a contact on a touch-sensitive surface). Contact intensity sensor 165 receives contact intensity information (e.g., pressure information or a proxy for pressure information) from the environment. In some embodiments, at least one contact intensity sensor is collocated with, or proximate to, a touch-sensitive surface (e.g., touch-sensitive display system 112). In some embodiments, at least one contact intensity sensor is located on the back of device 100, opposite touch screen display 112, which is located on the front of device 100.

Device 100 optionally also includes one or more proximity sensors 166. FIG. 1A shows proximity sensor 166 coupled to peripherals interface 118. Alternately, proximity sensor 166 is, optionally, coupled to input controller 160 in I/O subsystem 106. Proximity sensor 166 optionally performs as described in U.S. patent application Ser. No. 11/241,839, “Proximity Detector In Handheld Device”; Ser. No. 11/240,788, “Proximity Detector In Handheld Device”; Ser. No. 11/620,702, “Using Ambient Light Sensor To Augment Proximity Sensor Output”; Ser. No. 11/586,862, “Automated Response To And Sensing Of User Activity In Portable Devices”; and Ser. No. 11/638,251, “Methods And Systems For Automatic Configuration Of Peripherals,” which are hereby incorporated by reference in their entirety. In some embodiments, the proximity sensor turns off and disables touch screen 112 when the multifunction device is placed near the user's ear (e.g., when the user is making a phone call).

Device 100 optionally also includes one or more tactile output generators 167. FIG. 1A shows a tactile output generator coupled to haptic feedback controller 161 in I/O subsystem 106. Tactile output generator 167 optionally includes one or more electroacoustic devices such as speakers or other audio components and/or electromechanical devices that convert energy into linear motion such as a motor, solenoid, electroactive polymer, piezoelectric actuator, electrostatic actuator, or other tactile output generating component (e.g., a component that converts electrical signals into tactile outputs on the device). Contact intensity sensor 165 receives tactile feedback generation instructions from haptic feedback module 133 and generates tactile outputs on device 100 that are capable of being sensed by a user of device 100. In some embodiments, at least one tactile output generator is collocated with, or proximate to, a touch-sensitive surface (e.g., touch-sensitive display system 112) and, optionally, generates a tactile output by moving the touch-sensitive surface vertically (e.g., in/out of a surface of device 100) or laterally (e.g., back and forth in the same plane as a surface of device 100). In some embodiments, at least one tactile output generator sensor is located on the back of device 100, opposite touch screen display 112, which is located on the front of device 100.

Device 100 optionally also includes one or more accelerometers 168. FIG. 1A shows accelerometer 168 coupled to peripherals interface 118. Alternately, accelerometer 168 is, optionally, coupled to an input controller 160 in I/O subsystem 106. Accelerometer 168 optionally performs as described in U.S. Patent Publication No. 20050190059, “Acceleration-based Theft Detection System for Portable Electronic Devices,” and U.S. Patent Publication No. 20060017692, “Methods And Apparatuses For Operating A Portable Device Based On An Accelerometer,” both of which are incorporated by reference herein in their entirety. In some embodiments, information is displayed on the touch screen display in a portrait view or a landscape view based on an analysis of data received from the one or more accelerometers. Device 100 optionally includes, in addition to accelerometer(s) 168, a magnetometer and a GPS (or GLONASS or other global navigation system) receiver for obtaining information concerning the location and orientation (e.g., portrait or landscape) of device 100.

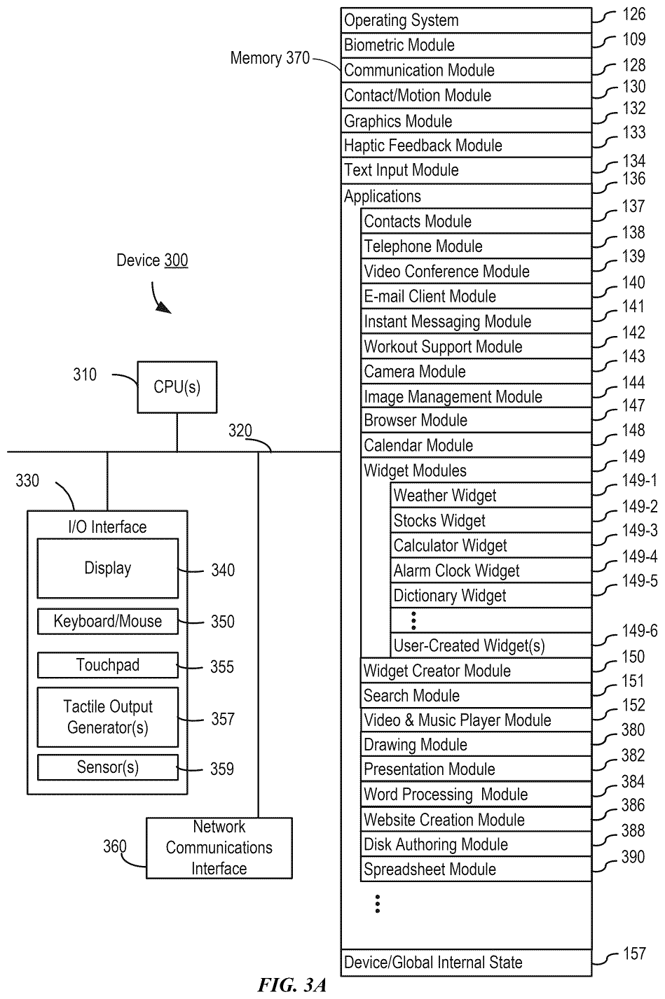

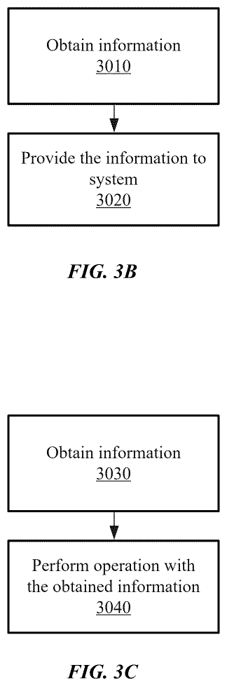

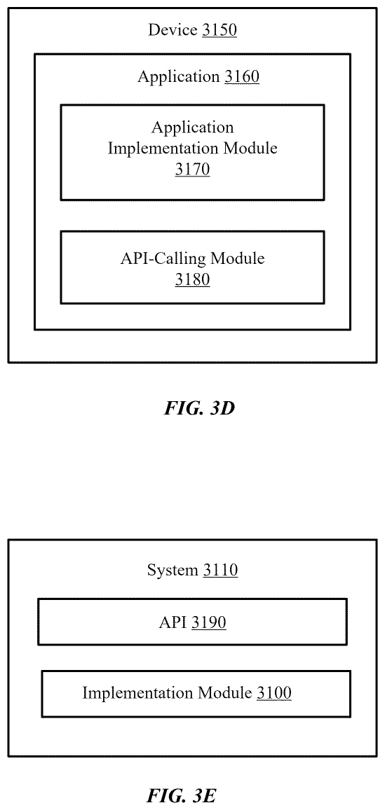

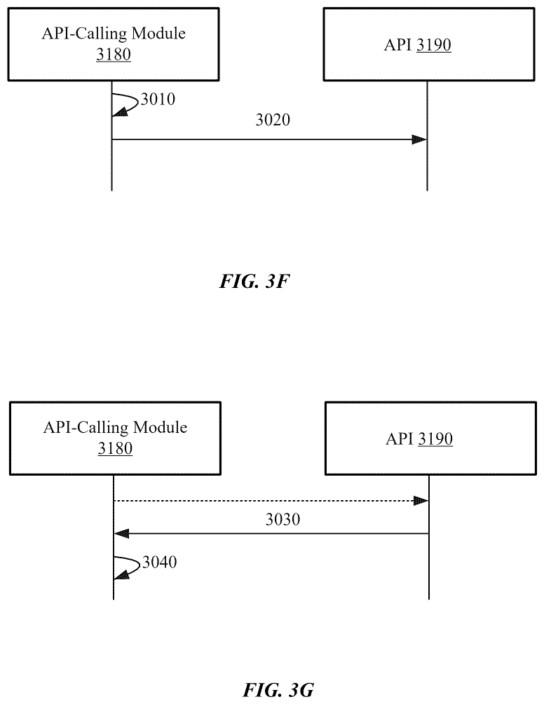

In some embodiments, the software components stored in memory 102 include operating system 126, biometric module 109, communication module (or set of instructions) 128, contact/motion module (or set of instructions) 130, graphics module (or set of instructions) 132, text input module (or set of instructions) 134, Global Positioning System (GPS) module (or set of instructions) 135, authentication module 105, and applications (or sets of instructions) 136. Furthermore, in some embodiments, memory 102 (FIG. 1A) or 370 (FIG. 3A) stores device/global internal state 157, as shown in FIGS. 1A and 3A. Device/global internal state 157 includes one or more of: active application state, indicating which applications, if any, are currently active; display state, indicating what applications, views or other information occupy various regions of touch screen display 112; sensor state, including information obtained from the device's various sensors and input control devices 116; and location information concerning the device's location and/or attitude.

Operating system 126 (e.g., Darwin, RTXC, LINUX, UNIX, OS X, IOS, WINDOWS, or an embedded operating system such as VxWorks) includes various software components and/or drivers for controlling and managing general system tasks (e.g., memory management, storage device control, power management, etc.) and facilitates communication between various hardware and software components.

Communication module 128 facilitates communication with other devices over one or more external ports 124 and also includes various software components for handling data received by RF circuitry 108 and/or external port 124. External port 124 (e.g., Universal Serial Bus (USB), FIREWIRE®, etc.) is adapted for coupling directly to other devices or indirectly over a network (e.g., the Internet, wireless LAN, etc.). In some embodiments, the external port is a multi-pin (e.g., 30-pin) connector that is the same as, or similar to and/or compatible with, the 30-pin connector used on iPod® (trademark of Apple Inc.) devices.

Biometric module 109 optionally stores information about one or more enrolled biometric features (e.g., fingerprint feature information, facial recognition feature information, eye and/or iris feature information) for use to verify whether received biometric information matches the enrolled biometric features. In some embodiments, the information stored about the one or more enrolled biometric features includes data that enables the comparison between the stored information and received biometric information without including enough information to reproduce the enrolled biometric features. In some embodiments, biometric module 109 stores the information about the enrolled biometric features in association with a user account of device 100. In some embodiments, biometric module 109 compares the received biometric information to an enrolled biometric feature to determine whether the received biometric information matches the enrolled biometric feature.

Contact/motion module 130 optionally detects contact with touch screen 112 (in conjunction with display controller 156) and other touch-sensitive devices (e.g., a touchpad or physical click wheel). Contact/motion module 130 includes various software components for performing various operations related to detection of contact, such as determining if contact has occurred (e.g., detecting a finger-down event), determining an intensity of the contact (e.g., the force or pressure of the contact or a substitute for the force or pressure of the contact), determining if there is movement of the contact and tracking the movement across the touch-sensitive surface (e.g., detecting one or more finger-dragging events), and determining if the contact has ceased (e.g., detecting a finger-up event or a break in contact). Contact/motion module 130 receives contact data from the touch-sensitive surface. Determining movement of the point of contact, which is represented by a series of contact data, optionally includes determining speed (magnitude), velocity (magnitude and direction), and/or an acceleration (a change in magnitude and/or direction) of the point of contact. These operations are, optionally, applied to single contacts (e.g., one finger contacts) or to multiple simultaneous contacts (e.g., “multitouch”/multiple finger contacts). In some embodiments, contact/motion module 130 and display controller 156 detect contact on a touchpad.

In some embodiments, contact/motion module 130 uses a set of one or more intensity thresholds to determine whether an operation has been performed by a user (e.g., to determine whether a user has “clicked” on an icon). In some embodiments, at least a subset of the intensity thresholds are determined in accordance with software parameters (e.g., the intensity thresholds are not determined by the activation thresholds of particular physical actuators and can be adjusted without changing the physical hardware of device 100). For example, a mouse “click” threshold of a trackpad or touch screen display can be set to any of a large range of predefined threshold values without changing the trackpad or touch screen display hardware. Additionally, in some implementations, a user of the device is provided with software settings for adjusting one or more of the set of intensity thresholds (e.g., by adjusting individual intensity thresholds and/or by adjusting a plurality of intensity thresholds at once with a system-level click “intensity” parameter).

Contact/motion module 130 optionally detects a gesture input by a user. Different gestures on the touch-sensitive surface have different contact patterns (e.g., different motions, timings, and/or intensities of detected contacts). Thus, a gesture is, optionally, detected by detecting a particular contact pattern. For example, detecting a finger tap gesture includes detecting a finger-down event followed by detecting a finger-up (liftoff) event at the same position (or substantially the same position) as the finger-down event (e.g., at the position of an icon). As another example, detecting a finger swipe gesture on the touch-sensitive surface includes detecting a finger-down event followed by detecting one or more finger-dragging events, and subsequently followed by detecting a finger-up (liftoff) event.

Graphics module 132 includes various known software components for rendering and displaying graphics on touch screen 112 or other display, including components for changing the visual impact (e.g., brightness, transparency, saturation, contrast, or other visual property) of graphics that are displayed. As used herein, the term “graphics” includes any object that can be displayed to a user, including, without limitation, text, web pages, icons (such as user-interface objects including soft keys), digital images, videos, animations, and the like.

In some embodiments, graphics module 132 stores data representing graphics to be used. Each graphic is, optionally, assigned a corresponding code. Graphics module 132 receives, from applications etc., one or more codes specifying graphics to be displayed along with, if necessary, coordinate data and other graphic property data, and then generates screen image data to output to display controller 156.

Haptic feedback module 133 includes various software components for generating instructions used by tactile output generator(s) 167 to produce tactile outputs at one or more locations on device 100 in response to user interactions with device 100.

Text input module 134, which is, optionally, a component of graphics module 132, provides soft keyboards for entering text in various applications (e.g., contacts module 137, e-mail client module 140, IM module 141, browser module 147, and any other application that needs text input).

GPS module 135 determines the location of the device and provides this information for use in various applications (e.g., to telephone module 138 for use in location-based dialing; to camera module 143 as picture/video metadata; and to applications that provide location-based services such as weather widgets, local yellow page widgets, and map/navigation widgets).

Authentication module 105 determines whether a requested operation (e.g., requested by an application of applications 136) is authorized to be performed. In some embodiments, authentication module 105 receives for an operation to be perform that optionally requires authentication. Authentication module 105 determines whether the operation is authorized to be performed, such as based on a series of factors, including the lock status of device 100, the location of device 100, whether a security delay has elapsed, whether received biometric information matches enrolled biometric features, and/or other factors. Once authentication module 105 determines that the operation is authorized to be performed, authentication module 105 triggers performance of the operation.

Applications 136 optionally include the following modules (or sets of instructions), or a subset or superset thereof:
Contacts module 137 (sometimes called an address book or contact list);
Telephone module 138;
Video conference module 139;
E-mail client module 140;
Instant messaging (IM) module 141;
Workout support module 142;
Camera module 143 for still and/or video images;
Image management module 144;
Video player module;
Music player module;
Browser module 147;
Calendar module 148;
Widget modules 149, which optionally include one or more of: weather widget 149-1, stocks widget 149-2, calculator widget 149-3, alarm clock widget 149-4, dictionary widget 149-5, and other widgets obtained by the user, as well as user-created widgets 149-6;
Widget creator module 150 for making user-created widgets 149-6;
Search module 151;
Video and music player module 152, which merges video player module and music player module;
Notes module 153;
Map module 154; and/or
Online video module 155.

Examples of other applications 136 that are, optionally, stored in memory 102 include other word processing applications, other image editing applications, drawing applications, presentation applications, JAVA-enabled applications, encryption, digital rights management, voice recognition, and voice replication.

In conjunction with touch screen 112, display controller 156, contact/motion module 130, graphics module 132, and text input module 134, contacts module 137 are, optionally, used to manage an address book or contact list (e.g., stored in application internal state 192 of contacts module 137 in memory 102 or memory 370), including: adding name(s) to the address book; deleting name(s) from the address book; associating telephone number(s), e-mail address(es), physical address(es) or other information with a name; associating an image with a name; categorizing and sorting names; providing telephone numbers or e-mail addresses to initiate and/or facilitate communications by telephone module 138, video conference module 139, e-mail client module 140, or IM module 141; and so forth.

In conjunction with RF circuitry 108, audio circuitry 110, speaker 111, microphone 113, touch screen 112, display controller 156, contact/motion module 130, graphics module 132, and text input module 134, telephone module 138 are optionally, used to enter a sequence of characters corresponding to a telephone number, access one or more telephone numbers in contacts module 137, modify a telephone number that has been entered, dial a respective telephone number, conduct a conversation, and disconnect or hang up when the conversation is completed. As noted above, the wireless communication optionally uses any of a plurality of communications standards, protocols, and technologies.

In conjunction with RF circuitry 108, audio circuitry 110, speaker 111, microphone 113, touch screen 112, display controller 156, optical sensor 164, optical sensor controller 158, contact/motion module 130, graphics module 132, text input module 134, contacts module 137, and telephone module 138, video conference module 139 includes executable instructions to initiate, conduct, and terminate a video conference between a user and one or more other participants in accordance with user instructions.

In conjunction with RF circuitry 108, touch screen 112, display controller 156, contact/motion module 130, graphics module 132, and text input module 134, e-mail client module 140 includes executable instructions to create, send, receive, and manage e-mail in response to user instructions. In conjunction with image management module 144, e-mail client module 140 makes it very easy to create and send e-mails with still or video images taken with camera module 143.

In conjunction with RF circuitry 108, touch screen 112, display controller 156, contact/motion module 130, graphics module 132, and text input module 134, the instant messaging module 141 includes executable instructions to enter a sequence of characters corresponding to an instant message, to modify previously entered characters, to transmit a respective instant message (for example, using a Short Message Service (SMS) or Multimedia Message Service (MMS) protocol for telephony-based instant messages or using XMPP, SIMPLE, or IMPS for Internet-based instant messages), to receive instant messages, and to view received instant messages. In some embodiments, transmitted and/or received instant messages optionally include graphics, photos, audio files, video files and/or other attachments as are supported in an MMS and/or an Enhanced Messaging Service (EMS). As used herein, “instant messaging” refers to both telephony-based messages (e.g., messages sent using SMS or MMS) and Internet-based messages (e.g., messages sent using XMPP, SIMPLE, or IMPS).

In conjunction with RF circuitry 108, touch screen 112, display controller 156, contact/motion module 130, graphics module 132, text input module 134, GPS module 135, map module 154, and music player module, workout support module 142 includes executable instructions to create workouts (e.g., with time, distance, and/or calorie burning goals); communicate with workout sensors (sports devices); receive workout sensor data; calibrate sensors used to monitor a workout; select and play music for a workout; and display, store, and transmit workout data.

In conjunction with touch screen 112, display controller 156, optical sensor(s) 164, optical sensor controller 158, contact/motion module 130, graphics module 132, and image management module 144, camera module 143 includes executable instructions to capture still images or video (including a video stream) and store them into memory 102, modify characteristics of a still image or video, or delete a still image or video from memory 102.

In conjunction with touch screen 112, display controller 156, contact/motion module 130, graphics module 132, text input module 134, and camera module 143, image management module 144 includes executable instructions to arrange, modify (e.g., edit), or otherwise manipulate, label, delete, present (e.g., in a digital slide show or album), and store still and/or video images.

In conjunction with RF circuitry 108, touch screen 112, display controller 156, contact/motion module 130, graphics module 132, and text input module 134, browser module 147 includes executable instructions to browse the Internet in accordance with user instructions, including searching, linking to, receiving, and displaying web pages or portions thereof, as well as attachments and other files linked to web pages.

In conjunction with RF circuitry 108, touch screen 112, display controller 156, contact/motion module 130, graphics module 132, text input module 134, e-mail client module 140, and browser module 147, calendar module 148 includes executable instructions to create, display, modify, and store calendars and data associated with calendars (e.g., calendar entries, to-do lists, etc.) in accordance with user instructions.

In conjunction with RF circuitry 108, touch screen 112, display controller 156, contact/motion module 130, graphics module 132, text input module 134, and browser module 147, widget modules 149 are mini-applications that are, optionally, downloaded and used by a user (e.g., weather widget 149-1, stocks widget 149-2, calculator widget 149-3, alarm clock widget 149-4, and dictionary widget 149-5) or created by the user (e.g., user-created widget 149-6). In some embodiments, a widget includes an HTML (Hypertext Markup Language) file, a CSS (Cascading Style Sheets) file, and a JavaScript® file. In some embodiments, a widget includes an XML (Extensible Markup Language) file and a JavaScript® file (e.g., Yahoo!® Widgets).

In conjunction with RF circuitry 108, touch screen 112, display controller 156, contact/motion module 130, graphics module 132, text input module 134, and browser module 147, the widget creator module 150 are, optionally, used by a user to create widgets (e.g., turning a user-specified portion of a web page into a widget).

In conjunction with touch screen 112, display controller 156, contact/motion module 130, graphics module 132, and text input module 134, search module 151 includes executable instructions to search for text, music, sound, image, video, and/or other files in memory 102 that match one or more search criteria (e.g., one or more user-specified search terms) in accordance with user instructions.

In conjunction with touch screen 112, display controller 156, contact/motion module 130, graphics module 132, audio circuitry 110, speaker 111, RF circuitry 108, and browser module 147, video and music player module 152 includes executable instructions that allow the user to download and play back recorded music and other sound files stored in one or more file formats, such as MP3 or AAC files, and executable instructions to display, present, or otherwise play back videos (e.g., on touch screen 112 or on an external, connected display via external port 124). In some embodiments, device 100 optionally includes the functionality of an MP3 player, such as an iPod (trademark of Apple Inc.).

In conjunction with touch screen 112, display controller 156, contact/motion module 130, graphics module 132, and text input module 134, notes module 153 includes executable instructions to create and manage notes, to-do lists, and the like in accordance with user instructions.

In conjunction with RF circuitry 108, touch screen 112, display controller 156, contact/motion module 130, graphics module 132, text input module 134, GPS module 135, and browser module 147, map module 154 are, optionally, used to receive, display, modify, and store maps and data associated with maps (e.g., driving directions, data on stores and other points of interest at or near a particular location, and other location-based data) in accordance with user instructions.

In conjunction with touch screen 112, display controller 156, contact/motion module 130, graphics module 132, audio circuitry 110, speaker 111, RF circuitry 108, text input module 134, e-mail client module 140, and browser module 147, online video module 155 includes instructions that allow the user to access, browse, receive (e.g., by streaming and/or download), play back (e.g., on the touch screen or on an external, connected display via external port 124), send an e-mail with a link to a particular online video, and otherwise manage online videos in one or more file formats, such as H.264. In some embodiments, instant messaging module 141, rather than e-mail client module 140, is used to send a link to a particular online video. Additional description of the online video application can be found in U.S. Provisional Patent Application No. 60/936,562, “Portable Multifunction Device, Method, and Graphical User Interface for Playing Online Videos,” filed Jun. 20, 2007, and U.S. patent application Ser. No. 11/968,067, “Portable Multifunction Device, Method, and Graphical User Interface for Playing Online Videos,” filed Dec. 31, 2007, the contents of which are hereby incorporated by reference in their entirety.

Each of the above-identified modules and applications corresponds to a set of executable instructions for performing one or more functions described above and the methods described in this application (e.g., the computer-implemented methods and other information processing methods described herein). These modules (e.g., sets of instructions) need not be implemented as separate software programs (such as computer programs (e.g., including instructions)), procedures, or modules, and thus various subsets of these modules are, optionally, combined or otherwise rearranged in various embodiments. For example, video player module is, optionally, combined with music player module into a single module (e.g., video and music player module 152, FIG. 1A). In some embodiments, memory 102 optionally stores a subset of the modules and data structures identified above. Furthermore, memory 102 optionally stores additional modules and data structures not described above.

In some embodiments, device 100 is a device where operation of a predefined set of functions on the device is performed exclusively through a touch screen and/or a touchpad. By using a touch screen and/or a touchpad as the primary input control device for operation of device 100, the number of physical input control devices (such as push buttons, dials, and the like) on device 100 is, optionally, reduced.

The predefined set of functions that are performed exclusively through a touch screen and/or a touchpad optionally include navigation between user interfaces. In some embodiments, the touchpad, when touched by the user, navigates device 100 to a main, home, or root menu from any user interface that is displayed on device 100. In such embodiments, a “menu button” is implemented using a touchpad. In some other embodiments, the menu button is a physical push button or other physical input control device instead of a touchpad.

FIG. 1B is a block diagram illustrating exemplary components for event handling in accordance with some embodiments. In some embodiments, memory 102 (FIG. 1A) or 370 (FIG. 3A) includes event sorter 170 (e.g., in operating system 126) and a respective application 136-1 (e.g., any of the aforementioned applications 137-151, 155, 380-390).

Event sorter 170 receives event information and determines the application 136-1 and application view 191 of application 136-1 to which to deliver the event information. Event sorter 170 includes event monitor 171 and event dispatcher module 174. In some embodiments, application 136-1 includes application internal state 192, which indicates the current application view(s) displayed on touch-sensitive display 112 when the application is active or executing. In some embodiments, device/global internal state 157 is used by event sorter 170 to determine which application(s) is (are) currently active, and application internal state 192 is used by event sorter 170 to determine application views 191 to which to deliver event information.

In some embodiments, application internal state 192 includes additional information, such as one or more of: resume information to be used when application 136-1 resumes execution, user interface state information that indicates information being displayed or that is ready for display by application 136-1, a state queue for enabling the user to go back to a prior state or view of application 136-1, and a redo/undo queue of previous actions taken by the user.

Event monitor 171 receives event information from peripherals interface 118. Event information includes information about a sub-event (e.g., a user touch on touch-sensitive display 112, as part of a multi-touch gesture). Peripherals interface 118 transmits information it receives from I/O subsystem 106 or a sensor, such as proximity sensor 166, accelerometer(s) 168, and/or microphone 113 (through audio circuitry 110). Information that peripherals interface 118 receives from I/O subsystem 106 includes information from touch-sensitive display 112 or a touch-sensitive surface.

In some embodiments, event monitor 171 sends requests to the peripherals interface 118 at predetermined intervals. In response, peripherals interface 118 transmits event information. In other embodiments, peripherals interface 118 transmits event information only when there is a significant event (e.g., receiving an input above a predetermined noise threshold and/or for more than a predetermined duration).

In some embodiments, event sorter 170 also includes a hit view determination module 172 and/or an active event recognizer determination module 173.

Hit view determination module 172 provides software procedures for determining where a sub-event has taken place within one or more views when touch-sensitive display 112 displays more than one view. Views are made up of controls and other elements that a user can see on the display.

Another aspect of the user interface associated with an application is a set of views, sometimes herein called application views or user interface windows, in which information is displayed and touch-based gestures occur. The application views (of a respective application) in which a touch is detected optionally correspond to programmatic levels within a programmatic or view hierarchy of the application. For example, the lowest level view in which a touch is detected is, optionally, called the hit view, and the set of events that are recognized as proper inputs are, optionally, determined based, at least in part, on the hit view of the initial touch that begins a touch-based gesture.

Hit view determination module 172 receives information related to sub-events of a touch-based gesture. When an application has multiple views organized in a hierarchy, hit view determination module 172 identifies a hit view as the lowest view in the hierarchy which should handle the sub-event. In most circumstances, the hit view is the lowest level view in which an initiating sub-event occurs (e.g., the first sub-event in the sequence of sub-events that form an event or potential event). Once the hit view is identified by the hit view determination module 172, the hit view typically receives all sub-events related to the same touch or input source for which it was identified as the hit view.

Active event recognizer determination module 173 determines which view or views within a view hierarchy should receive a particular sequence of sub-events. In some embodiments, active event recognizer determination module 173 determines that only the hit view should receive a particular sequence of sub-events. In other embodiments, active event recognizer determination module 173 determines that all views that include the physical location of a sub-event are actively involved views, and therefore determines that all actively involved views should receive a particular sequence of sub-events. In other embodiments, even if touch sub-events were entirely confined to the area associated with one particular view, views higher in the hierarchy would still remain as actively involved views.

Event dispatcher module 174 dispatches the event information to an event recognizer (e.g., event recognizer 180). In embodiments including active event recognizer determination module 173, event dispatcher module 174 delivers the event information to an event recognizer determined by active event recognizer determination module 173. In some embodiments, event dispatcher module 174 stores in an event queue the event information, which is retrieved by a respective event receiver 182.

In some embodiments, operating system 126 includes event sorter 170. Alternatively, application 136-1 includes event sorter 170. In yet other embodiments, event sorter 170 is a stand-alone module, or a part of another module stored in memory 102, such as contact/motion module 130.

In some embodiments, application 136-1 includes a plurality of event handlers 190 and one or more application views 191, each of which includes instructions for handling touch events that occur within a respective view of the application's user interface. Each application view 191 of the application 136-1 includes one or more event recognizers 180. Typically, a respective application view 191 includes a plurality of event recognizers 180. In other embodiments, one or more of event recognizers 180 are part of a separate module, such as a user interface kit or a higher level object from which application 136-1 inherits methods and other properties. In some embodiments, a respective event handler 190 includes one or more of: data updater 176, object updater 177, GUI updater 178, and/or event data 179 received from event sorter 170. Event handler 190 optionally utilizes or calls data updater 176, object updater 177, or GUI updater 178 to update the application internal state 192. Alternatively, one or more of the application views 191 include one or more respective event handlers 190. Also, in some embodiments, one or more of data updater 176, object updater 177, and GUI updater 178 are included in a respective application view 191.

A respective event recognizer 180 receives event information (e.g., event data 179) from event sorter 170 and identifies an event from the event information. Event recognizer 180 includes event receiver 182 and event comparator 184. In some embodiments, event recognizer 180 also includes at least a subset of: metadata 183, and event delivery instructions 188 (which optionally include sub-event delivery instructions).

Event receiver 182 receives event information from event sorter 170. The event information includes information about a sub-event, for example, a touch or a touch movement. Depending on the sub-event, the event information also includes additional information, such as location of the sub-event. When the sub-event concerns motion of a touch, the event information optionally also includes speed and direction of the sub-event. In some embodiments, events include rotation of the device from one orientation to another (e.g., from a portrait orientation to a landscape orientation, or vice versa), and the event information includes corresponding information about the current orientation (also called device attitude) of the device.

Event comparator 184 compares the event information to predefined event or sub-event definitions and, based on the comparison, determines an event or sub-event, or determines or updates the state of an event or sub-event. In some embodiments, event comparator 184 includes event definitions 186. Event definitions 186 contain definitions of events (e.g., predefined sequences of sub-events), for example, event 1 (187-1), event 2 (187-2), and others. In some embodiments, sub-events in an event (e.g., 187-1 and/or 187-2) include, for example, touch begin, touch end, touch movement, touch cancellation, and multiple touching. In one example, the definition for event 1 (187-1) is a double tap on a displayed object. The double tap, for example, comprises a first touch (touch begin) on the displayed object for a predetermined phase, a first liftoff (touch end) for a predetermined phase, a second touch (touch begin) on the displayed object for a predetermined phase, and a second liftoff (touch end) for a predetermined phase. In another example, the definition for event 2 (187-2) is a dragging on a displayed object. The dragging, for example, comprises a touch (or contact) on the displayed object for a predetermined phase, a movement of the touch across touch-sensitive display 112, and liftoff of the touch (touch end). In some embodiments, the event also includes information for one or more associated event handlers 190.

In some embodiments, event definitions 186 include a definition of an event for a respective user-interface object. In some embodiments, event comparator 184 performs a hit test to determine which user-interface object is associated with a sub-event. For example, in an application view in which three user-interface objects are displayed on touch-sensitive display 112, when a touch is detected on touch-sensitive display 112, event comparator 184 performs a hit test to determine which of the three user-interface objects is associated with the touch (sub-event). If each displayed object is associated with a respective event handler 190, the event comparator uses the result of the hit test to determine which event handler 190 should be activated. For example, event comparator 184 selects an event handler associated with the sub-event and the object triggering the hit test.

In some embodiments, the definition for a respective event (187-1) also includes delayed actions that delay delivery of the event information until after it has been determined whether the sequence of sub-events does or does not correspond to the event recognizer's event type.

When a respective event recognizer 180 determines that the series of sub-events do not match any of the events in event definitions 186, the respective event recognizer 180 enters an event impossible, event failed, or event ended state, after which it disregards subsequent sub-events of the touch-based gesture. In this situation, other event recognizers, if any, that remain active for the hit view continue to track and process sub-events of an ongoing touch-based gesture.

In some embodiments, a respective event recognizer 180 includes metadata 183 with configurable properties, flags, and/or lists that indicate how the event delivery system should perform sub-event delivery to actively involved event recognizers. In some embodiments, metadata 183 includes configurable properties, flags, and/or lists that indicate how event recognizers interact, or are enabled to interact, with one another. In some embodiments, metadata 183 includes configurable properties, flags, and/or lists that indicate whether sub-events are delivered to varying levels in the view or programmatic hierarchy.

In some embodiments, a respective event recognizer 180 activates event handler 190 associated with an event when one or more particular sub-events of an event are recognized. In some embodiments, a respective event recognizer 180 delivers event information associated with the event to event handler 190. Activating an event handler 190 is distinct from sending (and deferred sending) sub-events to a respective hit view. In some embodiments, event recognizer 180 throws a flag associated with the recognized event, and event handler 190 associated with the flag catches the flag and performs a predefined process.

In some embodiments, event delivery instructions 188 include sub-event delivery instructions that deliver event information about a sub-event without activating an event handler. Instead, the sub-event delivery instructions deliver event information to event handlers associated with the series of sub-events or to actively involved views. Event handlers associated with the series of sub-events or with actively involved views receive the event information and perform a predetermined process.

In some embodiments, data updater 176 creates and updates data used in application 136-1. For example, data updater 176 updates the telephone number used in contacts module 137, or stores a video file used in video player module. In some embodiments, object updater 177 creates and updates objects used in application 136-1. For example, object updater 177 creates a new user-interface object or updates the position of a user-interface object. GUI updater 178 updates the GUI. For example, GUI updater 178 prepares display information and sends it to graphics module 132 for display on a touch-sensitive display.

In some embodiments, event handler(s) 190 includes or has access to data updater 176, object updater 177, and GUI updater 178. In some embodiments, data updater 176, object updater 177, and GUI updater 178 are included in a single module of a respective application 136-1 or application view 191. In other embodiments, they are included in two or more software modules.

It shall be understood that the foregoing discussion regarding event handling of user touches on touch-sensitive displays also applies to other forms of user inputs to operate multifunction devices 100 with input devices, not all of which are initiated on touch screens. For example, mouse movement and mouse button presses, optionally coordinated with single or multiple keyboard presses or holds; contact movements such as taps, drags, scrolls, etc. on touchpads; pen stylus inputs; movement of the device; oral instructions; detected eye movements; biometric inputs; and/or any combination thereof are optionally utilized as inputs corresponding to sub-events which define an event to be recognized.

FIG. 2 illustrates a portable multifunction device 100 having a touch screen 112 in accordance with some embodiments. The touch screen optionally displays one or more graphics within user interface (UI) 200. In this embodiment, as well as others described below, a user is enabled to select one or more of the graphics by making a gesture on the graphics, for example, with one or more fingers 202 (not drawn to scale in the figure) or one or more styluses 203 (not drawn to scale in the figure). In some embodiments, selection of one or more graphics occurs when the user breaks contact with the one or more graphics. In some embodiments, the gesture optionally includes one or more taps, one or more swipes (from left to right, right to left, upward and/or downward), and/or a rolling of a finger (from right to left, left to right, upward and/or downward) that has made contact with device 100. In some implementations or circumstances, inadvertent contact with a graphic does not select the graphic. For example, a swipe gesture that sweeps over an application icon optionally does not select the corresponding application when the gesture corresponding to selection is a tap.

Device 100 optionally also include one or more physical buttons, such as “home” or menu button 204. As described previously, menu button 204 is, optionally, used to navigate to any application 136 in a set of applications that are, optionally, executed on device 100. Alternatively, in some embodiments, the menu button is implemented as a soft key in a GUI displayed on touch screen 112.

In some embodiments, device 100 includes touch screen 112, menu button 204, push button 206 for powering the device on/off and locking the device, volume adjustment button(s) 208, subscriber identity module (SIM) card slot 210, headset jack 212, and docking/charging external port 124. Push button 206 is, optionally, used to turn the power on/off on the device by depressing the button and holding the button in the depressed state for a predefined time interval; to lock the device by depressing the button and releasing the button before the predefined time interval has elapsed; and/or to unlock the device or initiate an unlock process. In an alternative embodiment, device 100 also accepts verbal input for activation or deactivation of some functions through microphone 113. Device 100 also, optionally, includes one or more contact intensity sensors 165 for detecting intensity of contacts on touch screen 112 and/or one or more tactile output generators 167 for generating tactile outputs for a user of device 100.

FIG. 3A is a block diagram of an exemplary multifunction device with a display and a touch-sensitive surface in accordance with some embodiments. Device 300 need not be portable. In some embodiments, device 300 is a laptop computer, a desktop computer, a tablet computer, a multimedia player device, a navigation device, an educational device (such as a child's learning toy), a gaming system, or a control device (e.g., a home or industrial controller). Device 300 typically includes one or more processing units (CPUs) 310, one or more network or other communications interfaces 360, memory 370, and one or more communication buses 320 for interconnecting these components. Communication buses 320 optionally include circuitry (sometimes called a chipset) that interconnects and controls communications between system components. Device 300 includes input/output (I/O) interface 330 comprising display 340, which is typically a touch screen display. I/O interface 330 also optionally includes a keyboard and/or mouse (or other pointing device) 350 and touchpad 355, tactile output generator 357 for generating tactile outputs on device 300 (e.g., similar to tactile output generator(s) 167 described above with reference to FIG. 1A), sensors 359 (e.g., optical, acceleration, proximity, touch-sensitive, and/or contact intensity sensors similar to contact intensity sensor(s) 165 described above with reference to FIG. 1A). Memory 370 includes high-speed random access memory, such as DRAM, SRAM, DDR RAM, or other random access solid state memory devices; and optionally includes non-volatile memory, such as one or more magnetic disk storage devices, optical disk storage devices, flash memory devices, or other non-volatile solid state storage devices. Memory 370 optionally includes one or more storage devices remotely located from CPU(s) 310. In some embodiments, memory 370 stores programs, modules, and data structures analogous to the programs, modules, and data structures stored in memory 102 of portable multifunction device 100 (FIG. 1A), or a subset thereof. Furthermore, memory 370 optionally stores additional programs, modules, and data structures not present in memory 102 of portable multifunction device 100. For example, memory 370 of device 300 optionally stores drawing module 380, presentation module 382, word processing module 384, website creation module 386, disk authoring module 388, and/or spreadsheet module 390, while memory 102 of portable multifunction device 100 (FIG. 1A) optionally does not store these modules.

Each of the above-identified elements in FIG. 3A is, optionally, stored in one or more of the previously mentioned memory devices. Each of the above-identified modules corresponds to a set of instructions for performing a function described above. The above-identified modules or computer programs (e.g., sets of instructions or including instructions) need not be implemented as separate software programs (such as computer programs (e.g., including instructions)), procedures, or modules, and thus various subsets of these modules are, optionally, combined or otherwise rearranged in various embodiments. In some embodiments, memory 370 optionally stores a subset of the modules and data structures identified above. Furthermore, memory 370 optionally stores additional modules and data structures not described above.

Implementations within the scope of the present disclosure can be partially or entirely realized using a tangible computer-readable storage medium (or multiple tangible computer-readable storage media of one or more types) encoding one or more computer-readable instructions. It should be recognized that computer-readable instructions can be organized in any format, including applications, widgets, processes, software, and/or components.

Implementations within the scope of the present disclosure include a computer-readable storage medium that encodes instructions organized as an application (e.g., application 3160) that, when executed by one or more processing units, control an electronic device (e.g., device 3150) to perform the method of FIG. 3B, the method of FIG. 3C, and/or one or more other processes and/or methods described herein.

It should be recognized that application 3160 (shown in FIG. 3D) can be any suitable type of application, including, for example, one or more of: a browser application, an application that functions as an execution environment for plug-ins, widgets or other applications, a fitness application, a health application, a digital payments application, a media application, a social network application, a messaging application, and/or a maps application. In some embodiments, application 3160 is an application that is pre-installed on device 3150 at purchase (e.g., a first-party application). In some embodiments, application 3160 is an application that is provided to device 3150 via an operating system update file (e.g., a first-party application or a second-party application). In some embodiments, application 3160 is an application that is provided via an application store. In some embodiments, the application store can be an application store that is pre-installed on device 3150 at purchase (e.g., a first-party application store). In some embodiments, the application store is a third-party application store (e.g., an application store that is provided by another application store, downloaded via a network, and/or read from a storage device).

Referring to FIG. 3B and FIG. 3F, application 3160 obtains information (e.g., 3010). In some embodiments, at 3010, information is obtained from at least one hardware component of device 3150. In some embodiments, at 3010, information is obtained from at least one software module of device 3150. In some embodiments, at 3010, information is obtained from at least one hardware component external to device 3150 (e.g., a peripheral device, an accessory device, and/or a server). In some embodiments, the information obtained at 3010 includes positional information, time information, notification information, user information, environment information, electronic device state information, weather information, media information, historical information, event information, hardware information, and/or motion information. In some embodiments, in response to and/or after obtaining the information at 3010, application 3160 provides the information to a system (e.g., 3020).

In some embodiments, the system (e.g., 3110 shown in FIG. 3E) is an operating system hosted on device 3150. In some embodiments, the system (e.g., 3110 shown in FIG. 3E) is an external device (e.g., a server, a peripheral device, an accessory, and/or a personal computing device) that includes an operating system.

Referring to FIG. 3C and FIG. 3G, application 3160 obtains information (e.g., 3030). In some embodiments, the information obtained at 3030 includes positional information, time information, notification information, user information, environment information electronic device state information, weather information, media information, historical information, event information, hardware information, and/or motion information. In response to and/or after obtaining the information at 3030, application 3160 performs an operation with the information (e.g., 3040). In some embodiments, the operation performed at 3040 includes: providing a notification based on the information, sending a message based on the information, displaying the information, controlling a user interface of a fitness application based on the information, controlling a user interface of a health application based on the information, controlling a focus mode based on the information, setting a reminder based on the information, adding a calendar entry based on the information, and/or calling an API of system 3110 based on the information.

In some embodiments, one or more steps of the method of FIG. 3B and/or the method of FIG. 3C is performed in response to a trigger. In some embodiments, the trigger includes detection of an event, a notification received from system 3110, a user input, and/or a response to a call to an API provided by system 3110.

In some embodiments, the instructions of application 3160, when executed, control device 3150 to perform the method of FIG. 3B and/or the method of FIG. 3C by calling an application programming interface (API) (e.g., API 3190) provided by system 3110. In some embodiments, application 3160 performs at least a portion of the method of FIG. 3B and/or the method of FIG. 3C without calling API 3190.

In some embodiments, one or more steps of the method of FIG. 3B and/or the method of FIG. 3C includes calling an API (e.g., API 3190) using one or more parameters defined by the API. In some embodiments, the one or more parameters include a constant, a key, a data structure, an object, an object class, a variable, a data type, a pointer, an array, a list or a pointer to a function or method, and/or another way to reference a data or other item to be passed via the API.

Referring to FIG. 3D, device 3150 is illustrated. In some embodiments, device 3150 is a personal computing device, a smart phone, a smart watch, a fitness tracker, a head mounted display (HM D) device, a media device, a communal device, a speaker, a television, and/or a tablet. As illustrated in FIG. 3D, device 3150 includes application 3160 and an operating system (e.g., system 3110 shown in FIG. 3E). Application 3160 includes application implementation module 3170 and API-calling module 3180. System 3110 includes API 3190 and implementation module 3100. It should be recognized that device 3150, application 3160, and/or system 3110 can include more, fewer, and/or different components than illustrated in FIGS. 3D and 3E.

In some embodiments, application implementation module 3170 includes a set of one or more instructions corresponding to one or more operations performed by application 3160. For example, when application 3160 is a messaging application, application implementation module 3170 can include operations to receive and send messages. In some embodiments, application implementation module 3170 communicates with API-calling module 3180 to communicate with system 3110 via API 3190 (shown in FIG. 3E).

In some embodiments, API 3190 is a software module (e.g., a collection of computer-readable instructions) that provides an interface that allows a different module (e.g., API-calling module 3180) to access and/or use one or more functions, methods, procedures, data structures, classes, and/or other services provided by implementation module 3100 of system 3110. For example, API-calling module 3180 can access a feature of implementation module 3100 through one or more API calls or invocations (e.g., embodied by a function or a method call) exposed by API 3190 (e.g., a software and/or hardware module that can receive API calls, respond to API calls, and/or send API calls) and can pass data and/or control information using one or more parameters via the API calls or invocations. In some embodiments, API 3190 allows application 3160 to use a service provided by a Software Development Kit (SDK) library. In some embodiments, application 3160 incorporates a call to a function or method provided by the SDK library and provided by API 3190 or uses data types or objects defined in the SDK library and provided by API 3190. In some embodiments, API-calling module 3180 makes an API call via API 3190 to access and use a feature of implementation module 3100 that is specified by API 3190. In such embodiments, implementation module 3100 can return a value via API 3190 to API-calling module 3180 in response to the API call. The value can report to application 3160 the capabilities or state of a hardware component of device 3150, including those related to aspects such as input capabilities and state, output capabilities and state, processing capability, power state, storage capacity and state, and/or communications capability. In some embodiments, API 3190 is implemented in part by firmware, microcode, or other low level logic that executes in part on the hardware component.

In some embodiments, API 3190 allows a developer of API-calling module 3180 (which can be a third-party developer) to leverage a feature provided by implementation module 3100. In such embodiments, there can be one or more API-calling modules (e.g., including API-calling module 3180) that communicate with implementation module 3100. In some embodiments, API 3190 allows multiple API-calling modules written in different programming languages to communicate with implementation module 3100 (e.g., API 3190 can include features for translating calls and returns between implementation module 3100 and API-calling module 3180) while API 3190 is implemented in terms of a specific programming language. In some embodiments, API-calling module 3180 calls APIs from different providers such as a set of APIs from an OS provider, another set of APIs from a plug-in provider, and/or another set of APIs from another provider (e.g., the provider of a software library) or creator of the another set of APIs.

Examples of API 3190 can include one or more of: a pairing API (e.g., for establishing secure connection, e.g., with an accessory), a device detection API (e.g., for locating nearby devices, e.g., media devices and/or smartphone), a payment API, a UIK it API (e.g., for generating user interfaces), a location detection API, a locator API, a maps API, a health sensor API, a sensor API, a messaging API, a push notification API, a streaming API, a collaboration API, a video conferencing API, an application store API, an advertising services API, a web browser API (e.g., WebKit API), a vehicle API, a networking API, a WiFi API, a Bluetooth API, an NFC API, a UWB API, a fitness API, a smart home API, contact transfer API, photos API, camera API, and/or image processing API. In some embodiments, the sensor API is an API for accessing data associated with a sensor of device 3150. For example, the sensor API can provide access to raw sensor data. For another example, the sensor API can provide data derived (and/or generated) from the raw sensor data. In some embodiments, the sensor data includes temperature data, image data, video data, audio data, heart rate data, IM U (inertial measurement unit) data, lidar data, location data, GPS data, and/or camera data. In some embodiments, the sensor includes one or more of an accelerometer, temperature sensor, infrared sensor, optical sensor, heartrate sensor, barometer, gyroscope, proximity sensor, temperature sensor, and/or biometric sensor.

In some embodiments, implementation module 3100 is a system (e.g., operating system and/or server system) software module (e.g., a collection of computer-readable instructions) that is constructed to perform an operation in response to receiving an API call via API 3190. In some embodiments, implementation module 3100 is constructed to provide an API response (via API 3190) as a result of processing an API call. By way of example, implementation module 3100 and API-calling module 3180 can each be any one of an operating system, a library, a device driver, an API, an application program, or other module. It should be understood that implementation module 3100 and API-calling module 3180 can be the same or different type of module from each other. In some embodiments, implementation module 3100 is embodied at least in part in firmware, microcode, or hardware logic.

In some embodiments, implementation module 3100 returns a value through API 3190 in response to an API call from API-calling module 3180. While API 3190 defines the syntax and result of an API call (e.g., how to invoke the API call and what the API call does), API 3190 might not reveal how implementation module 3100 accomplishes the function specified by the API call. Various API calls are transferred via the one or more application programming interfaces between API-calling module 3180 and implementation module 3100. Transferring the API calls can include issuing, initiating, invoking, calling, receiving, returning, and/or responding to the function calls or messages. In other words, transferring can describe actions by either of API-calling module 3180 or implementation module 3100. In some embodiments, a function call or other invocation of API 3190 sends and/or receives one or more parameters through a parameter list or other structure.

In some embodiments, implementation module 3100 provides more than one API, each providing a different view of or with different aspects of functionality implemented by implementation module 3100. For example, one API of implementation module 3100 can provide a first set of functions and can be exposed to third-party developers, and another API of implementation module 3100 can be hidden (e.g., not exposed) and provide a subset of the first set of functions and also provide another set of functions, such as testing or debugging functions which are not in the first set of functions. In some embodiments, implementation module 3100 calls one or more other components via an underlying API and thus is both an API-calling module and an implementation module. It should be recognized that implementation module 3100 can include additional functions, methods, classes, data structures, and/or other features that are not specified through API 3190 and are not available to API-calling module 3180. It should also be recognized that API-calling module 3180 can be on the same system as implementation module 3100 or can be located remotely and access implementation module 3100 using API 3190 over a network. In some embodiments, implementation module 3100, API 3190, and/or API-calling module 3180 is stored in a machine-readable medium, which includes any mechanism for storing information in a form readable by a machine (e.g., a computer or other data processing system). For example, a machine-readable medium can include magnetic disks, optical disks, random access memory; read only memory, and/or flash memory devices.

An application programming interface (API) is an interface between a first software process and a second software process that specifies a format for communication between the first software process and the second software process. Limited APIs (e.g., private APIs or partner APIs) are APIs that are accessible to a limited set of software processes (e.g., only software processes within an operating system or only software processes that are approved to access the limited APIs). Public APIs that are accessible to a wider set of software processes. Some APIs enable software processes to communicate about or set a state of one or more input devices (e.g., one or more touch sensors, proximity sensors, visual sensors, motion/orientation sensors, pressure sensors, intensity sensors, sound sensors, wireless proximity sensors, biometric sensors, buttons, switches, rotatable elements, and/or external controllers). Some APIs enable software processes to communicate about and/or set a state of one or more output generation components (e.g., one or more audio output generation components, one or more display generation components, and/or one or more tactile output generation components). Some APIs enable particular capabilities (e.g., scrolling, handwriting, text entry, image editing, and/or image creation) to be accessed, performed, and/or used by a software process (e.g., generating outputs for use by a software process based on input from the software process). Some APIs enable content from a software process to be inserted into a template and displayed in a user interface that has a layout and/or behaviors that are specified by the template.

Many software platforms include a set of frameworks that provides the core objects and core behaviors that a software developer needs to build software applications that can be used on the software platform. Software developers use these objects to display content onscreen, to interact with that content, and to manage interactions with the software platform. Software applications rely on the set of frameworks for their basic behavior, and the set of frameworks provides many ways for the software developer to customize the behavior of the application to match the specific needs of the software application. Many of these core objects and core behaviors are accessed via an API. An API will typically specify a format for communication between software processes, including specifying and grouping available variables, functions, and protocols. An API call (sometimes referred to as an API request) will typically be sent from a sending software process to a receiving software process as a way to accomplish one or more of the following: the sending software process requesting information from the receiving software process (e.g., for the sending software process to take action on), the sending software process providing information to the receiving software process (e.g., for the receiving software process to take action on), the sending software process requesting action by the receiving software process, or the sending software process providing information to the receiving software process about action taken by the sending software process. Interaction with a device (e.g., using a user interface) will in some circumstances include the transfer and/or receipt of one or more API calls (e.g., multiple API calls) between multiple different software processes (e.g., different portions of an operating system, an application and an operating system, or different applications) via one or more APIs (e.g., via multiple different APIs). For example, when an input is detected the direct sensor data is frequently processed into one or more input events that are provided (e.g., via an API) to a receiving software process that makes some determination based on the input events, and then sends (e.g., via an API) information to a software process to perform an operation (e.g., change a device state and/or user interface) based on the determination. While a determination and an operation performed in response could be made by the same software process, alternatively the determination could be made in a first software process and relayed (e.g., via an API) to a second software process, that is different from the first software process, that causes the operation to be performed by the second software process. Alternatively, the second software process could relay instructions (e.g., via an API) to a third software process that is different from the first software process and/or the second software process to perform the operation. It should be understood that some or all user interactions with a computer system could involve one or more API calls within a step of interacting with the computer system (e.g., between different software components of the computer system or between a software component of the computer system and a software component of one or more remote computer systems). It should be understood that some or all user interactions with a computer system could involve one or more API calls between steps of interacting with the computer system (e.g., between different software components of the computer system or between a software component of the computer system and a software component of one or more remote computer systems).

In some embodiments, the application can be any suitable type of application, including, for example, one or more of: a browser application, an application that functions as an execution environment for plug-ins, widgets or other applications, a fitness application, a health application, a digital payments application, a media application, a social network application, a messaging application, and/or a maps application.

In some embodiments, the application is an application that is pre-installed on the first computer system at purchase (e.g., a first-party application). In some embodiments, the application is an application that is provided to the first computer system via an operating system update file (e.g., a first-party application). In some embodiments, the application is an application that is provided via an application store. In some embodiments, the application store is pre-installed on the first computer system at purchase (e.g., a first-party application store) and allows download of one or more applications. In some embodiments, the application store is a third-party application store (e.g., an application store that is provided by another device, downloaded via a network, and/or read from a storage device). In some embodiments, the application is a third-party application (e.g., an app that is provided by an application store, downloaded via a network, and/or read from a storage device). In some embodiments, the application controls the first computer system to perform methods 700, 900, and/or 1100 (FIGS. 7, 9, and/or 11) by calling an application programming interface (API) provided by the system process using one or more parameters.

In some embodiments, exemplary APIs provided by the system process include one or more of: a pairing API (e.g., for establishing secure connection, e.g., with an accessory), a device detection API (e.g., for locating nearby devices, e.g., media devices and/or smartphone), a payment API, a UIK it API (e.g., for generating user interfaces), a location detection API, a locator API, a maps API, a health sensor API, a sensor API, a messaging API, a push notification API, a streaming API, a collaboration API, a video conferencing API, an application store API, an advertising services API, a web browser API (e.g., WebKit API), a vehicle API, a networking API, a WiFi API, a Bluetooth API, an NFC API, a UWB API, a fitness API, a smart home API, contact transfer API, a photos API, a camera API, and/or an image processing API.

In some embodiments, at least one API is a software module (e.g., a collection of computer-readable instructions) that provides an interface that allows a different module (e.g., API-calling module 3180) to access and use one or more functions, methods, procedures, data structures, classes, and/or other services provided by an implementation module of the system process. The API can define one or more parameters that are passed between the API-calling module and the implementation module. In some embodiments, API 3190 defines a first API call that can be provided by API-calling module 3180. The implementation module is a system software module (e.g., a collection of computer-readable instructions) that is constructed to perform an operation in response to receiving an API call via the API. In some embodiments, the implementation module is constructed to provide an API response (via the API) as a result of processing an API call. In some embodiments, the implementation module is included in the device (e.g., 3150) that runs the application. In some embodiments, the implementation module is included in an electronic device that is separate from the device that runs the application.

Attention is now directed towards embodiments of user interfaces that are, optionally, implemented on, for example, portable multifunction device 100.

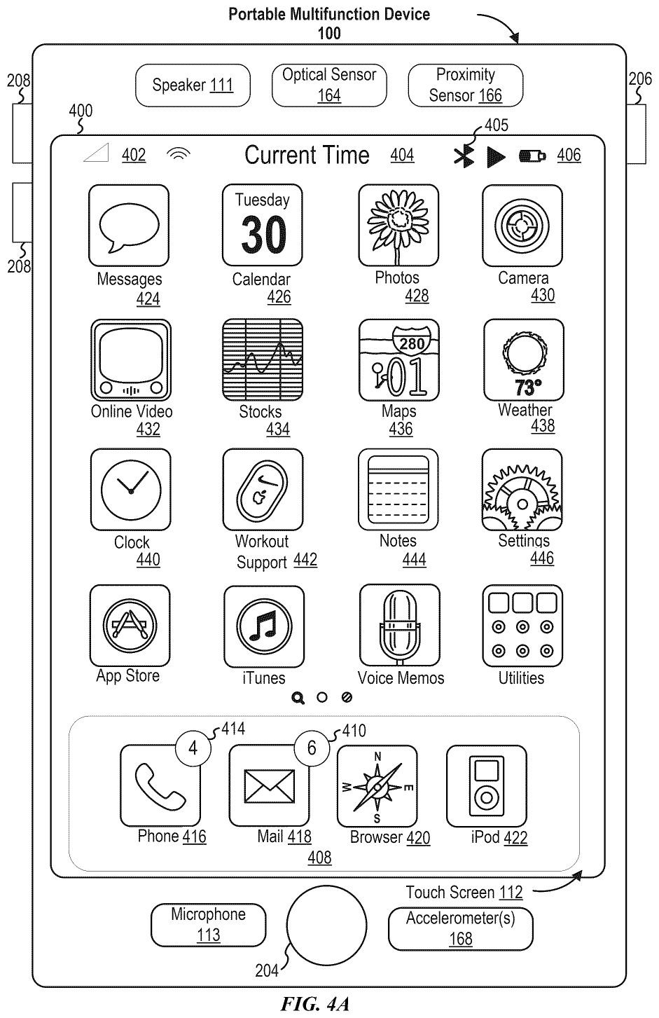

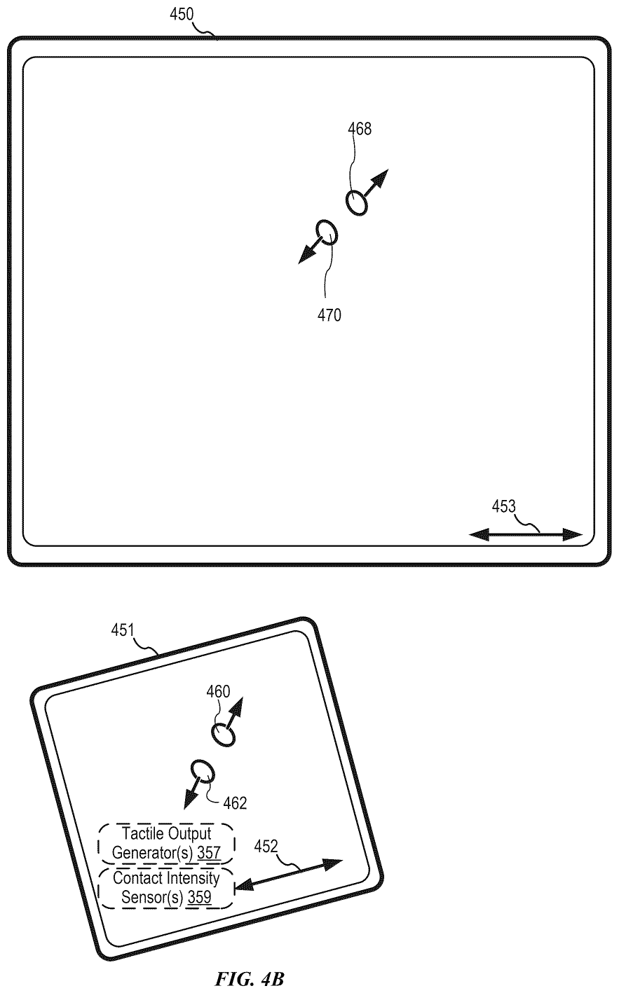

FIG. 4A illustrates an exemplary user interface for a menu of applications on portable multifunction device 100 in accordance with some embodiments. Similar user interfaces are, optionally, implemented on device 300. In some embodiments, user interface 400 includes the following elements, or a subset or superset thereof:
Signal strength indicator(s) 402 for wireless communication(s), such as cellular and Wi-Fi signals;
Time 404;
Bluetooth indicator 405;
Battery status indicator 406;
Tray 408 with icons for frequently used applications, such as:
   Icon 416 for telephone module 138, labeled “Phone,” which optionally includes an indicator 414 of the number of missed calls or voicemail messages;
   Icon 418 for e-mail client module 140, labeled “Mail,” which optionally includes an indicator 410 of the number of unread e-mails;
   Icon 420 for browser module 147, labeled “Browser;” and
   Icon 422 for video and music player module 152, also referred to as iPod (trademark of Apple Inc.) module 152, labeled “iPod;” and
Icons for other applications, such as:
   Icon 424 for IM module 141, labeled “Messages;”
   Icon 426 for calendar module 148, labeled “Calendar;”
   Icon 428 for image management module 144, labeled “Photos;”
   Icon 430 for camera module 143, labeled “Camera;”
   Icon 432 for online video module 155, labeled “Online Video;”
   Icon 434 for stocks widget 149-2, labeled “Stocks;”
   Icon 436 for map module 154, labeled “Maps;”
   Icon 438 for weather widget 149-1, labeled “Weather;”
   Icon 440 for alarm clock widget 149-4, labeled “Clock;”
   Icon 442 for workout support module 142, labeled “Workout Support;”
   Icon 444 for notes module 153, labeled “Notes;” and
   Icon 446 for a settings application or module, labeled “Settings,” which provides access to settings for device 100 and its various applications 136.

It should be noted that the icon labels illustrated in FIG. 4A are merely exemplary. For example, icon 422 for video and music player module 152 is labeled “Music” or “Music Player.” Other labels are, optionally, used for various application icons. In some embodiments, a label for a respective application icon includes a name of an application corresponding to the respective application icon. In some embodiments, a label for a particular application icon is distinct from a name of an application corresponding to the particular application icon.

FIG. 4B illustrates an exemplary user interface on a device (e.g., device 300, FIG. 3A) with a touch-sensitive surface 451 (e.g., a tablet or touchpad 355, FIG. 3A) that is separate from the display 450 (e.g., touch screen display 112). Device 300 also, optionally, includes one or more contact intensity sensors (e.g., one or more of sensors 359) for detecting intensity of contacts on touch-sensitive surface 451 and/or one or more tactile output generators 357 for generating tactile outputs for a user of device 300.

Although some of the examples that follow will be given with reference to inputs on touch screen display 112 (where the touch-sensitive surface and the display are combined), in some embodiments, the device detects inputs on a touch-sensitive surface that is separate from the display, as shown in FIG. 4B. In some embodiments, the touch-sensitive surface (e.g., touch-sensitive surface 451 in FIG. 4B) has a primary axis (e.g., 452 in FIG. 4B) that corresponds to a primary axis (e.g., 453 in FIG. 4B) on the display (e.g., display 450). In accordance with these embodiments, the device detects contacts (e.g., contact 460 and contact 462 in FIG. 4B) with the touch-sensitive surface 451 at locations that correspond to respective locations on the display (e.g., in FIG. 4B, contact 460 corresponds to 468 and contact 462 corresponds to 470). In this way, user inputs (e.g., contacts 460 and 462, and movements thereof) detected by the device on the touch-sensitive surface (e.g., touch-sensitive surface 451 in FIG. 4B) are used by the device to manipulate the user interface on the display (e.g., display 450 in FIG. 4B) of the multifunction device when the touch-sensitive surface is separate from the display. It should be understood that similar methods are, optionally, used for other user interfaces described herein.

Additionally, while the following examples are given primarily with reference to finger inputs (e.g., finger contacts, finger tap gestures, finger swipe gestures), it should be understood that, in some embodiments, one or more of the finger inputs are replaced with input from another input device (e.g., a mouse-based input or stylus input). For example, a swipe gesture is, optionally, replaced with a mouse click (e.g., instead of a contact) followed by movement of the cursor along the path of the swipe (e.g., instead of movement of the contact). As another example, a tap gesture is, optionally, replaced with a mouse click while the cursor is located over the location of the tap gesture (e.g., instead of detection of the contact followed by ceasing to detect the contact). Similarly, when multiple user inputs are simultaneously detected, it should be understood that multiple computer mice are, optionally, used simultaneously, or a mouse and finger contacts are, optionally, used simultaneously.

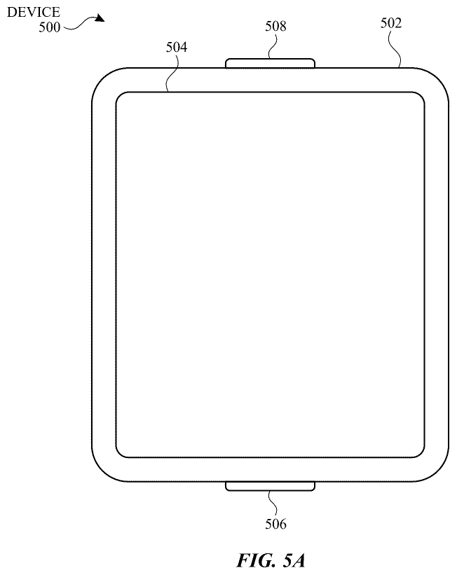

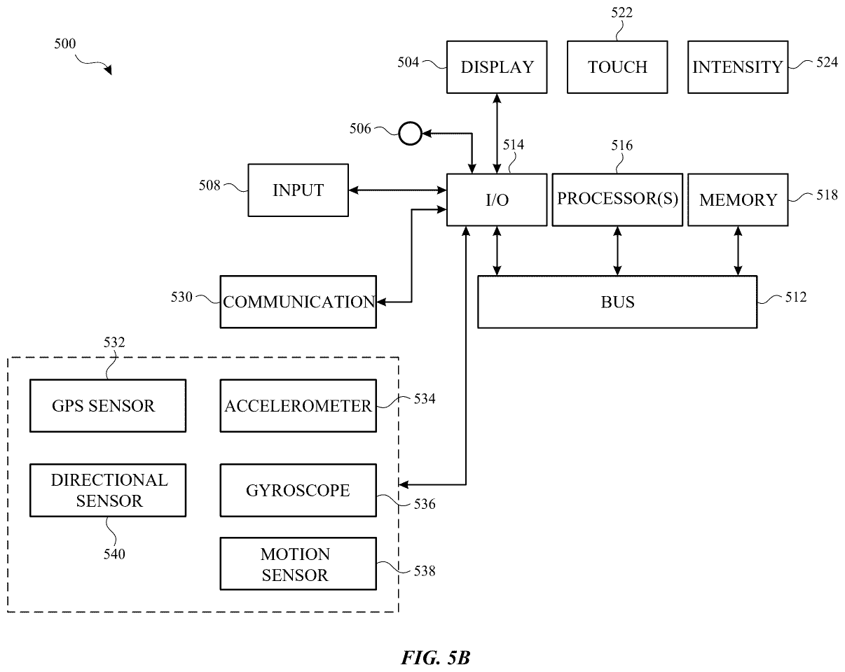

FIG. 5A illustrates exemplary personal electronic device 500. Device 500 includes body 502. In some embodiments, device 500 can include some or all of the features described with respect to devices 100 and 300 (e.g., FIGS. 1A-4B). In some embodiments, device 500 has touch-sensitive display screen 504, hereafter touch screen 504. Alternatively, or in addition to touch screen 504, device 500 has a display and a touch-sensitive surface. As with devices 100 and 300, in some embodiments, touch screen 504 (or the touch-sensitive surface) optionally includes one or more intensity sensors for detecting intensity of contacts (e.g., touches) being applied. The one or more intensity sensors of touch screen 504 (or the touch-sensitive surface) can provide output data that represents the intensity of touches. The user interface of device 500 can respond to touches based on their intensity, meaning that touches of different intensities can invoke different user interface operations on device 500.

Exemplary techniques for detecting and processing touch intensity are found, for example, in related applications: International Patent Application Serial No. PCT/US2013/040061, titled “Device, Method, and Graphical User Interface for Displaying User Interface Objects Corresponding to an Application,” filed May 8, 2013, published as WIPO Publication No. WO/2013/169849, and International Patent Application Serial No. PCT/US2013/069483, titled “Device, Method, and Graphical User Interface for Transitioning Between Touch Input to Display Output Relationships,” filed Nov. 11, 2013, published as WIPO Publication No. WO/2014/105276, each of which is hereby incorporated by reference in their entirety.

In some embodiments, device 500 has one or more input mechanisms 506 and 508. Input mechanisms 506 and 508, if included, can be physical. Examples of physical input mechanisms include push buttons and rotatable mechanisms. In some embodiments, device 500 has one or more attachment mechanisms. Such attachment mechanisms, if included, can permit attachment of device 500 with, for example, hats, eyewear, earrings, necklaces, shirts, jackets, bracelets, watch straps, chains, trousers, belts, shoes, purses, backpacks, and so forth. These attachment mechanisms permit device 500 to be worn by a user.

FIG. 5B depicts exemplary personal electronic device 500. In some embodiments, device 500 can include some or all of the components described with respect to FIGS. 1A, 1B, and 3A. Device 500 has bus 512 that operatively couples I/O section 514 with one or more computer processors 516 and memory 518. I/O section 514 can be connected to display screen 504, which can have touch-sensitive component 522 and, optionally, intensity sensor 524 (e.g., contact intensity sensor). In addition, I/O section 514 can be connected with communication unit 530 for receiving application and operating system data, using Wi-Fi, Bluetooth, near field communication (NFC), cellular, and/or other wireless communication techniques. Device 500 can include input mechanisms 506 and/or 508. Input mechanism 506 is, optionally, a rotatable input device or a depressible and rotatable input device, for example. Input mechanism 508 is, optionally, a button, in some examples.

Input mechanism 508 is, optionally, a microphone, in some examples. Personal electronic device 500 optionally includes various sensors, such as GPS sensor 532, accelerometer 534, directional sensor 540 (e.g., compass), gyroscope 536, motion sensor 538, and/or a combination thereof, all of which can be operatively connected to I/O section 514.

Memory 518 of personal electronic device 500 can include one or more non-transitory computer-readable storage media, for storing computer-executable instructions, which, when executed by one or more computer processors 516, for example, can cause the computer processors to perform the techniques described below, including processes 700, 900, and 1100 (FIGS. 7, 9, and 11). A computer-readable storage medium can be any medium that can tangibly contain or store computer-executable instructions for use by or in connection with the instruction execution system, apparatus, or device. In some examples, the storage medium is a transitory computer-readable storage medium. In some examples, the storage medium is a non-transitory computer-readable storage medium. The non-transitory computer-readable storage medium can include, but is not limited to, magnetic, optical, and/or semiconductor storages. Examples of such storage include magnetic disks, optical discs based on CD, DVD, or Blu-ray technologies, as well as persistent solid-state memory such as flash, solid-state drives, and the like. Personal electronic device 500 is not limited to the components and configuration of FIG. 5B, but can include other or additional components in multiple configurations.

As used here, the term “affordance” refers to a user-interactive graphical user interface object that is, optionally, displayed on the display screen of devices 100, 300, and/or 500 (FIGS. 1A, 3A, and 5A-5B). For example, an image (e.g., icon), a button, and text (e.g., hyperlink) each optionally constitute an affordance.

As used herein, the term “focus selector” refers to an input element that indicates a current part of a user interface with which a user is interacting. In some implementations that include a cursor or other location marker, the cursor acts as a “focus selector” so that when an input (e.g., a press input) is detected on a touch-sensitive surface (e.g., touchpad 355 in FIG. 3A or touch-sensitive surface 451 in FIG. 4B) while the cursor is over a particular user interface element (e.g., a button, window, slider, or other user interface element), the particular user interface element is adjusted in accordance with the detected input. In some implementations that include a touch screen display (e.g., touch-sensitive display system 112 in FIG. 1A or touch screen 112 in FIG. 4A) that enables direct interaction with user interface elements on the touch screen display, a detected contact on the touch screen acts as a “focus selector” so that when an input (e.g., a press input by the contact) is detected on the touch screen display at a location of a particular user interface element (e.g., a button, window, slider, or other user interface element), the particular user interface element is adjusted in accordance with the detected input. In some implementations, focus is moved from one region of a user interface to another region of the user interface without corresponding movement of a cursor or movement of a contact on a touch screen display (e.g., by using a tab key or arrow keys to move focus from one button to another button); in these implementations, the focus selector moves in accordance with movement of focus between different regions of the user interface. Without regard to the specific form taken by the focus selector, the focus selector is generally the user interface element (or contact on a touch screen display) that is controlled by the user so as to communicate the user's intended interaction with the user interface (e.g., by indicating, to the device, the element of the user interface with which the user is intending to interact). For example, the location of a focus selector (e.g., a cursor, a contact, or a selection box) over a respective button while a press input is detected on the touch-sensitive surface (e.g., a touchpad or touch screen) will indicate that the user is intending to activate the respective button (as opposed to other user interface elements shown on a display of the device).

As used in the specification and claims, the term “characteristic intensity” of a contact refers to a characteristic of the contact based on one or more intensities of the contact. In some embodiments, the characteristic intensity is based on multiple intensity samples. The characteristic intensity is, optionally, based on a predefined number of intensity samples, or a set of intensity samples collected during a predetermined time period (e.g., 0.05, 0.1, 0.2, 0.5, 1, 2, 5, 10 seconds) relative to a predefined event (e.g., after detecting the contact, prior to detecting liftoff of the contact, before or after detecting a start of movement of the contact, prior to detecting an end of the contact, before or after detecting an increase in intensity of the contact, and/or before or after detecting a decrease in intensity of the contact). A characteristic intensity of a contact is, optionally, based on one or more of: a maximum value of the intensities of the contact, a mean value of the intensities of the contact, an average value of the intensities of the contact, a top 10 percentile value of the intensities of the contact, a value at the half maximum of the intensities of the contact, a value at the 90 percent maximum of the intensities of the contact, or the like. In some embodiments, the duration of the contact is used in determining the characteristic intensity (e.g., when the characteristic intensity is an average of the intensity of the contact over time). In some embodiments, the characteristic intensity is compared to a set of one or more intensity thresholds to determine whether an operation has been performed by a user. For example, the set of one or more intensity thresholds optionally includes a first intensity threshold and a second intensity threshold. In this example, a contact with a characteristic intensity that does not exceed the first threshold results in a first operation, a contact with a characteristic intensity that exceeds the first intensity threshold and does not exceed the second intensity threshold results in a second operation, and a contact with a characteristic intensity that exceeds the second threshold results in a third operation. In some embodiments, a comparison between the characteristic intensity and one or more thresholds is used to determine whether or not to perform one or more operations (e.g., whether to perform a respective operation or forgo performing the respective operation), rather than being used to determine whether to perform a first operation or a second operation.

Attention is now directed towards embodiments of user interfaces (“UI”) and associated processes that are implemented on an electronic device, such as portable multifunction device 100, device 300, or device 500.

FIGS. 6A-6W illustrate exemplary user interfaces for managing notifications related to a pregnancy event, in accordance with some embodiments. The user interfaces in these figures are used to illustrate the processes described below, including the processes in FIG. 7.

FIG. 6A illustrates computer system 600 displaying, via display 602, health user interface 604. At FIG. 6A, health user interface 604 corresponds to a health application of computer system 600. For example, health user interface 604 includes selectable options 604a-604j corresponding to respective health information of an account that is signed into and/or otherwise corresponds to computer system 600. In some embodiments, computer system 600 receives information related to the health of the user of computer system 600 and logs and/or records the health information, such as heart rate and/or activity information (e.g., a number of steps, an amount of exercise minutes, an estimated number of calories burned over a respective duration, and/or a distance traveled over a respective duration). In some embodiments, computer system 600 prompts a user and/or enables a user of computer system 600 to input health information (e.g., via one or more user interfaces of the health application corresponding to health user interface 604). In some embodiments, in response to user input corresponding to a respective selectable option 604a-604j, computer system 600 displays one or more respective user interfaces that include and/or enable a user to input a respective type of health information. For example, in some embodiments, in response to detecting user input corresponding to selectable option 604e, computer system 600 displays a user interface that includes information about a heart rate of the user of computer system 600 and/or a user interface that enables a user to input a blood pressure measurement.

At FIG. 6A, computer system 600 detects user input 650a (e.g., a tap input, a touch input, an air gesture, a mouse click, a gaze input, and/or a speech input) corresponding to selectable option 604c. As set forth below, selectable option 604c corresponds to cycle tracking health information. In response to detecting user input 650a, computer system 600 displays cycle tracking user interface 606, as shown at FIG. 6B. Alternatively, computer system 600 detects user input 650b (e.g., a tap input, a touch input, an air gesture, a mouse click, a gaze input, and/or a speech input) corresponding to account user interface object 604k of health user interface 604. In response to detecting user input 650b, computer system 600 displays account user interface 608, as shown at FIG. 6E.

At FIG. 6B, computer system 600 displays cycle tracking user interface 606, which includes information related to a menstrual cycle of a user corresponding to the account of computer system 600. In some embodiments, the user of computer system 600 establishes an account that is logged into and/or otherwise accessible by computer system 600. Computer system 600 is configured to track, store, and/or otherwise receive information corresponding to health information of the user while the account corresponds to (e.g., is logged into, is authorized for use with, and/or is accessible by) computer system 600. In some embodiments, the user of computer system 600 configures and/or authorizes computer system 600 to collect, track, receive, predict, estimate, and/or display information related to the menstrual cycle of the user.

At FIG. 6B, cycle tracking user interface 606 includes cycle tracking region 610, which includes cycle tracking indicators 610a-610i corresponding to respective days. Cycle tracking indicators 610a-610i provide visual indications of information about the menstrual cycle of the user of the account corresponding to computer system 600. For example, cycle tracking indicators 610a-610i visually indicate whether the user was and/or is currently experiencing a menstrual cycle on a respective day, whether the user experienced a menstrual cycle symptom on a respective day, whether the user experienced bleeding on a respective day, and/or fertility information for a respective day. In some embodiments, computer system 600 receives information (e.g., via one or more user inputs) indicating that the user of the account corresponding to computer system 600 is pregnant. As set forth below, in some embodiments, cycle tracking region 610 includes cycle tracking indicators that include different appearances from cycle tracking indicators 610a-610i and/or provide visual indications of information related to the pregnancy.

At FIG. 6B, cycle tracking indicator 610e is currently selected, as indicated by computer system 600 displaying cycle tracking indicator 610e at a larger size as compared to cycle tracking indicators 610a-610d and/or 610f-610i. At FIG. 6B, cycle tracking indicator 610e corresponds to the current day (e.g., the current day at which computer system 600 displays cycle tracking user interface 606). In some embodiments, computer system 600 displays cycle tracking user interface 606 with a different cycle tracking indicator 610a-610i selected (e.g., a cycle tracking indicator that does not correspond to the current day). In some embodiments, computer system 600 is configured to add and/or associate menstrual cycle information with a respective cycle tracking indicator 610a-610i in response to detecting selection of a respective cycle tracking indicator 610a-610i. For example, in some embodiments, in response to detecting selection of a respective cycle tracking indicator 610a-610i while the account corresponding to computer system 600 does not include a pregnancy designation (e.g., the user of the account has not provided information indicating that they are pregnant), computer system 600 displays the respective cycle tracking indicator 610a-610i with a menstrual cycle indicator. In some embodiments, computer system 600 determines, logs, records, tracks, and/or otherwise includes information that the user of the account experienced and/or is experiencing their menstrual cycle on the respective day corresponding to the selected cycle tracking indicator 610a-610i. In some embodiments, in response to detecting selection of a respective cycle tracking indicator 610a-610i while the account corresponding to computer system 600 includes a pregnancy designation, computer system 600 displays a bleeding indicator, a symptom indicator, and/or another user interface.

At FIG. 6B, cycle tracking user interface 606 includes period user interface object 606a, symptom user interface object 606b, spotting user interface object 606c, and factors user interface object 606d. In some embodiments, computer system 600 determines, logs, records, and/or otherwise includes information that the user experienced and/or is experiencing their menstrual cycle on the respective day corresponding to the selected cycle tracking indicator 610a-610i (e.g., cycle tracking indicator 610e at FIG. 6B) in response to detecting selection of period user interface object 606a. In some embodiments, computer system 600 displays a menstrual cycle user interface in response to detecting selection of period user interface object 606a. In some embodiments, computer system 600 displays a symptoms user interface (e.g., a user interface that includes selectable options corresponding to symptoms that a user experiences during a menstrual cycle) in response to detecting selection of symptom user interface object 606b and/or displays a symptom indicator on the selected cycle tracking indicator 610a-610i. In some embodiments, computer system 600 displays a spotting user interface (e.g., a user interface with selectable spotting/bleeding options that a user experiences during a menstrual cycle) in response to detecting selection of spotting user interface object 606c and/or displays a spotting indicator on the selected cycle tracking indicator 610a-610i. As set forth below, in response to detecting selection of factors user interface object 606d, computer system 600 displays factors user interface 612, as shown at FIG. 6C.

At FIG. 6B, computer system 600 displays pregnancy designation user interface object 606e on cycle tracking user interface 606. In some embodiments, computer system 600 displays pregnancy designation user interface object 606e based on information received by computer system 600 (e.g., a positive pregnancy test result). In some embodiments, computer system 600 displays pregnancy designation user interface object 606e based on information related to the menstrual cycle of the user of the account (e.g., the user has indicated that they have not experienced their menstrual cycle for a threshold amount of time). In some embodiments, computer system 600 displays pregnancy designation user interface object 606e as a default user interface object of cycle tracking user interface 606 (e.g., computer system 600 displays pregnancy designation user interface object 606e unless computer system 600 receives user input requesting to dismiss, clear, and/or remove display of pregnancy designation user interface object 606e). At FIG. 6B, computer system 600 detects user input 650c (e.g., a tap input, a touch input, an air gesture, a mouse click, a gaze input, and/or a speech input) corresponding to selection of pregnancy designation user interface object 606e. In response to detecting user input 650c, computer system 600 initiates a process for adding a pregnancy event and/or pregnancy designation to the account corresponding to computer system 600 and displays pregnancy user interface 614, as shown at FIG. 6G.

Alternatively, at FIG. 6B, computer system 600 detects user input 650d (e.g., a tap input, a touch input, an air gesture, a mouse click, a gaze input, and/or a speech input) corresponding to selection of factors user interface object 606d of cycle tracking user interface 606. In response to detecting user input 650d, computer system 600 displays factors user interface 612, as shown at FIG. 6C.

At FIG. 6C, factors user interface 612 enables computer system 600 to add life factors to the account corresponding to computer system 600 in response to one or more user inputs. For instance, factors user interface 612 allows computer system 600 to add and/or track additional information about a menstrual cycle of the user. At FIG. 6C, factors user interface 612 indicates that computer system 600 has not received any information about life factors of the user and/or added a life factor to the account for the current date (e.g., the date at which computer system 600 displays factors user interface 612), as illustrated by current factor user interface object 612a. Factors user interface 612 also includes show all factors user interface object 612b. In some embodiments, in response to detecting selection of show all factors user interface object 612b, computer system 600 displays another user interface and/or user interface objects including information related to life factors that the user of computer system 600 experienced in the past.

At FIG. 6C, computer system 600 detects user input 650e (e.g., a tap input, a touch input, an air gesture, a mouse click, a gaze input, and/or a speech input) corresponding to selection of add factor user interface object 612c. In response to detecting user input 650e, computer system 600 displays new factor user interface 616, as shown at FIG. 6D.

At FIG. 6D, computer system 600 displays new factor user interface 616, which includes selectable options 616a-616c that correspond to respective life factors that are related to a menstrual cycle of the user. For instance, selectable option 616a corresponds to a pregnancy life factor, selectable option 616b corresponds to a lactation life factor, and selectable option 616c corresponds to a contraceptive life factor. New factor user interface 616 also includes start user interface object 616d that enables computer system 600 to receive information about when the user started to experience the respective factor. In addition, new factor user interface 616 includes end user interface object 616e that allows computer system 600 to receive information about when the user stopped experiencing the respective factor. As shown at FIG. 6D, end user interface object 616e includes a designation of “Not Yet,” which allows a user to select an option indicating that the user is still experiencing the respective factor.

At FIG. 6D, computer system 600 detects user input 650f (e.g., a tap input, a touch input, an air gesture, a mouse click, a gaze input, and/or a speech input) corresponding to selectable option 616a. In some embodiments, in response to detecting user input 650f, computer system 600 displays a selection indicator (e.g., a check mark and/or an emphasis) corresponding to selectable option 616a to confirm selection of selectable option 616a. In some embodiments, computer system 600 receives one or more additional user inputs corresponding to start user interface object 616d and/or end user interface object 616e before or after detecting user input 650f. After detecting user input 650f, computer system 600 detects user input 650g (e.g., a tap input, a touch input, an air gesture, a mouse click, a gaze input, and/or a speech input) corresponding to done user interface object 616f of new factor user interface 616. In response to detecting user input 650f and user input 650g, computer system 600 initiates the process for adding a pregnancy event and/or pregnancy designation to the account corresponding to computer system 600 and displays pregnancy user interface 614, as shown at FIG. 6G.

As set forth above, computer system 600 displays account user interface 608 in response to detecting user input 650b corresponding to selection of account user interface object 604k of health user interface 604, as shown at FIG. 6E. At FIG. 6E, account user interface 608 includes one or more selectable options corresponding to respective user interfaces for configuring, customizing, and/or managing the account corresponding to computer system 600. At FIG. 6E, computer system 600 detects user input 650h (e.g., a tap input, a touch input, an air gesture, a mouse click, a gaze input, and/or a speech input) corresponding to health details user interface object 608a of account user interface 608. In response to detecting user input 650h, computer system 600 displays health details user interface 618, as shown at FIG. 6F.

At FIG. 6F, health details user interface 618 includes information about the user of the account corresponding to computer system 600. For instance, health details user interface 618 includes date of birth indicator 618a, sex indicator 618b, height indicator 618c, and weight indicator 618d. In some embodiments, computer system 600 receives information (e.g., via one or more inputs) corresponding to the information about the user of the account corresponding to computer system 600 and displays and/or updates indicators 618a-618d based on the received information. In some embodiments, indicators 618a-618d relate to information about the user of the account corresponding to computer system 600 that relate to health and/or activity of the user.

At FIG. 6F, health details user interface 618 includes add pregnancy user interface object 618e that, when selected via user input, initiates a process to add a pregnancy event and/or pregnancy designation to the account. At FIG. 6F, computer system 600 detects user input 650i (e.g., a tap input, a touch input, an air gesture, a mouse click, a gaze input, and/or a speech input) corresponding to add pregnancy user interface object 618e. In response to detecting user input 650i, computer system 600 initiates the process for adding a pregnancy event and/or pregnancy designation to the account corresponding to computer system 600 and displays pregnancy user interface 614, as shown at FIG. 6G.

Accordingly, computer system 600 provides different user interfaces and/or user interface objects that allow computer system 600 to initiate the process for adding the pregnancy event and/or pregnancy designation to the account, as set forth above with reference to FIGS. 6A-6F. At FIG. 6G, computer system 600 displays pregnancy user interface 614 in response to detecting user input(s) 650c, 650f, 650g, and/or 650i.

At FIG. 6G, pregnancy user interface 614 provides an option to estimate an age (e.g., a gestational age) and/or due date of a pregnancy event. For example, pregnancy user interface 614 includes estimation method user interface object 614a and estimated due date user interface object 614b. As set forth below, computer system 600 displays one or more options for estimating the age and/or due date of the pregnancy event in response to detecting user input (e.g., user input 650j) corresponding to selection of estimation method user interface object 614a. In some embodiments, computer system 600 updates an appearance and/or information included in estimated due date user interface object 614b in response to receiving information related to the pregnancy event.

At FIG. 6G, pregnancy user interface 614 includes next user interface object 614c. In some embodiments, in response to detecting selection of next user interface object 614c, and without detecting one or more user inputs corresponding to estimation method user interface object 614a and/or estimated due date user interface object 614b, computer system 600 skips a process for estimating the age and/or due date of the pregnancy event and displays add pregnancy user interface 620, as shown at FIG. 6J. Pregnancy user interface 614 also includes past pregnancy user interface object 614d. In some embodiments, in response to detecting user input corresponding to past pregnancy user interface object 614d, computer system 600 displays one or more user interfaces that prompt the user of computer system 600 to provide information about a previous pregnancy (e.g., a pregnancy that has already resulted in birth and/or otherwise ended), such as a start date, an end date, and/or a birth date.

At FIG. 6G, computer system 600 detects user input 650j (e.g., a tap input, a touch input, an air gesture, a mouse click, a gaze input, and/or a speech input) corresponding to estimation method user interface object 614a. In response to detecting user input 650j, computer system 600 displays estimation user interface 622, as shown at FIG. 6H.

At FIG. 6H, estimation user interface 622 includes selectable options 622a-622d corresponding to different techniques, information, methods, and/or ways to estimate the age and/or due date of the pregnancy. As such, the user of computer system 600 can determine which method to select based on available information in order to receive an estimated age and/or due date for the pregnancy event. For instance, selectable option 622a corresponds to a last menstrual period date that allows computer system 600 to determine and/or estimate the age and/or due date of the pregnancy event based on the first day of the last menstrual cycle of the user of the account corresponding to computer system 600. Selectable option 622b corresponds to an estimated due date option that allows computer system 600 to determine and/or estimate the age and/or due date of the pregnancy event based on a start date of the pregnancy event (e.g., a start date known by the user and/or estimated by the user and/or a doctor of the user). Selectable option 622c corresponds to an estimated age of the pregnancy event that allows computer system 600 to determine and/or estimate the age and/or due date of the pregnancy event based on an amount of time that the user has been pregnant (e.g., an amount of time known by the user and/or estimated by the user and/or a doctor of the user). Further, selectable option 622d relates to an embryo transfer date option that allows computer system 600 to determine and/or estimate the age and/or due date of the pregnancy event based on a date at which an embryo was transferred via an invitro fertilization procedure.

At FIG. 6H, computer system 600 detects user input 650k (e.g., a tap input, a touch input, an air gesture, a mouse click, a gaze input, and/or a speech input) corresponding to selection of selectable option 622c. In response to detecting user input 650k, computer system 600 displays (e.g., re-displays) pregnancy user interface 614, as shown at FIG. 6I.

At FIG. 6I, computer system 600 displays pregnancy user interface 614, which includes age user interface object 614e and start date user interface object 614f. In some embodiments, computer system 600 is configured to estimate the age, the start date, and/or the due date of the pregnancy event based on information received from the user (e.g., via user input). For instance, at FIG. 6I, computer system 600 detects user input 650l (e.g., a tap input, a touch input, an air gesture, a mouse click, a gaze input, and/or a speech input) corresponding to age user interface object 614e. In some embodiments, in response to detecting user input 650l, computer system 600 displays a menu, a keyboard, a calendar, and/or one or more selectable options that allow a user to select and/or input a current and/or estimated duration of the pregnancy event. For instance, at FIG. 6I, computer system 600 has detected one or more user inputs corresponding to the current and/or estimated duration of the pregnancy event being 5 weeks and 1 day.

Alternatively, in some embodiments, computer system 600 detects user input 650m (e.g., a tap input, a touch input, an air gesture, a mouse click, a gaze input, and/or a speech input) corresponding to start date user interface object 614f. In some embodiments, in response to detecting user input 650m, computer system 600 displays a menu, keyboard, a calendar, and/or one or more selectable options that allow a user to select and/or input a start date of the pregnancy event. In some embodiments, computer system 600 does not detect user input 650m, and computer system 600 estimates, determines, and/or calculates the start date of the pregnancy event based on the information related to the current duration of the pregnancy event received via user input 650l (and, optionally, one or more additional user inputs). At FIG. 6I, computer system 600 displays a start date of Aug. 20, 2024 as the start date of the pregnancy event based on user input 650m (and, optionally, one or more additional user inputs) and/or based on the information related to the current duration of the pregnancy event.

In response to detecting user input 650l and/or 650m, computer system 600 updates display of estimated due date user interface object 614b, as shown at FIG. 6I. For instance, computer system 600 estimates, determines, and/or approximates a due date of the pregnancy event based on information received via user input 650l and/or 650m. At FIG. 6I, computer system 600 updates estimated due date user interface object 614b to indicate that the estimated, determined, and/or approximate due date of the pregnancy event is May 10, 2025.

In some embodiments, computer system 600 receives information about an estimated due date of the pregnancy event (e.g., computer system 600 detects one or more inputs corresponding to estimated due date user interface object 614b that include information related an estimated due date received from a doctor of the user and/or estimated by the user). In some embodiments, computer system 600 estimates the age of the pregnancy based on information received about the estimated due date of the pregnancy event.

At FIG. 6I, computer system 600 detects user input 650n (e.g., a tap input, a touch input, an air gesture, a mouse click, a gaze input, and/or a speech input) (e.g., after detecting user input 650l and/or user input 650m) corresponding to next user interface object 614c of pregnancy user interface 614. In response to detecting user input 650n, computer system 600 displays add pregnancy user interface 620, as shown at FIG. 6J.

At FIG. 6J, add pregnancy user interface 620 is an introductory user interface that computer system 600 displays to confirm that the user of computer system 600 intends to add the pregnancy event to the account corresponding to computer system 600. For instance, add pregnancy user interface 620 includes information region 620a, which includes information and/or details about the process for adding the pregnancy event to the account corresponding to computer system 600. At FIG. 6J, computer system 600 detects user input 6500 (e.g., a tap input, a touch input, an air gesture, a mouse click, a gaze input, and/or a speech input) corresponding to next user interface object 620b of add pregnancy user interface 620. In response to detecting user input 6500, computer system 600 displays walking steadiness user interface 624, as shown at FIG. 6K.

At FIG. 6K, walking steadiness user interface 624 provides one or more options for managing and/or configuring notifications related to walking steadiness. In some embodiments, computer system 600 is configured to receive information and/or determine the walking steadiness of the user based on an estimated stability of the user while walking. In some embodiments, computer system 600 determines and/or estimates the walking steadiness of the user while walking based on a walking speed of the user, a step length of the user, a double support time of the user, and/or a walking asymmetry of the user. In some embodiments, the walking steadiness of the user is indicative of a fall risk of the user, which can increase while the user is pregnant. In some embodiments, computer system 600 outputs notifications when the determined and/or estimated walking steadiness of the user falls below a threshold value. In some embodiments, computer system 600 is configured to activate and/or deactivate walking steadiness notifications in response to user input. In some embodiments, computer system 600 displays walking steadiness user interface 624 based on a determination that walking steadiness notifications are deactivated (e.g., deactivated at a time at which computer system 600 initiates the process to add the pregnancy event to the account corresponding to computer system 600). In some embodiments, computer system 600 displays walking steadiness user interface 624 regardless of whether walking steadiness notifications are activated or deactivated.

At FIG. 6K, walking steadiness user interface 624 includes information region 624a, activation user interface object 624b, and skip user interface object 624c. Information region 624a includes text, images, icons, and/or graphical user interface objects that provide the user of computer system 600 with information about walking steadiness notifications and/or why activating walking steadiness notifications may be beneficial to the user while the user is pregnant. For instance, at FIG. 6K, information region 624a includes text indicating that the walking steadiness of the user may decrease when the user is pregnant, which can increase a risk of the user falling. At FIG. 6K, computer system 600 detects user input 650p (e.g., a tap input, a touch input, an air gesture, a mouse click, a gaze input, and/or a speech input) corresponding to activation user interface object 624b. In response to detecting user input 650p, computer system 600 initiates a process for activating walking steadiness notifications and displays heart rate user interface 626, as shown at FIG. 6L. Alternatively, at FIG. 6K, computer system 600 detects user input 650q (e.g., a tap input, a touch input, an air gesture, a mouse click, a gaze input, and/or a speech input) corresponding to skip user interface object 624c. In some embodiments, in response to detecting user input 650q, computer system 600 displays heart rate user interface 626 without initiating the process for activating walking steadiness notifications.

At FIG. 6L, heart rate user interface 626 provides one or more options for managing and/or configuring notifications related to the heart rate of the user. In some embodiments, computer system 600 is configured to receive information about, determine, and/or estimate the heart rate of the user. In some embodiments, computer system 600 determines and/or estimates the heart rate of the user based on information received from an external device (e.g., a smart watch that is paired to and/or in communication with computer system 600 and/or a heart rate sensor that is in communication with computer system 600). In some embodiments, computer system 600 outputs notifications when the received, determined, and/or estimated heart rate of the user falls below and/or exceeds a threshold value. In some embodiments, computer system 600 adjusts, changes, and/or modifies the threshold value of the heart rate of the user based on user input, based on an age of the user, and/or based on contextual factors of the user (e.g., whether the user is pregnant and/or has a heart condition). In some embodiments, computer system 600 is configured to activate and/or deactivate heart rate notifications in response to user input. In some embodiments, computer system 600 displays heart rate user interface 626 based on a determination that heart rate notifications are deactivated (e.g., deactivated at a time at which computer system 600 initiates the process to add the pregnancy event to the account corresponding to computer system 600). In some embodiments, computer system 600 displays heart rate user interface 626 regardless of whether heart rate notifications are activated or deactivated.

At FIG. 6L, heart rate user interface 626 includes information region 626a, activation user interface object 626b, and skip user interface object 626c. Information region 626a includes text, images, icons, and/or graphical user interface objects that provide the user of computer system 600 with information about heart rate notifications, information about why heart rate notifications may be beneficial to the user while the user is pregnant, and/or information about adjusting a heart rate threshold for heart rate notifications while the user is pregnant. For instance, at FIG. 6L, information region 626a includes text indicating that heart rate increases during pregnancy and that activating heart rate notifications during the pregnancy event results in computer system 600 applying and/or using a higher heart rate threshold for heart rate notifications. At FIG. 6L, computer system 600 detects user input 650r (e.g., a tap input, a touch input, an air gesture, a mouse click, a gaze input, and/or a speech input) corresponding to activation user interface object 626b. In response to detecting user input 650r, computer system 600 initiates a process for activating heart rate notifications, modifies and/or adjusts the heart rate threshold for the heart rate notifications, and displays cardio fitness user interface 628, as shown at FIG. 6M. Alternatively, at FIG. 6L, computer system 600 detects user input 650s (e.g., a tap input, a touch input, an air gesture, a mouse click, a gaze input, and/or a speech input) corresponding to skip user interface object 626c. In some embodiments, in response to detecting user input 650s, computer system 600 displays cardio fitness user interface 628 without initiating the process for activating heart rate notifications and/or modifying the heart rate threshold for the heart rate notifications.

At FIG. 6M, cardio fitness user interface 628 provides one or more options for managing and/or configuring notifications related to the cardio fitness and/or VO₂ max of the user. In some embodiments, computer system 600 is configured to receive information about, determine, and/or estimate the cardio fitness of the user. In some embodiments, computer system 600 determines and/or estimates the cardio fitness of the user based on information received from an external device (e.g., a smart watch that is paired to and/or in communication with computer system 600), such as heart rate information and/or motion information. In some embodiments, computer system 600 outputs notifications when the determined and/or estimated cardio fitness of the user falls below a threshold value. In some embodiments, computer system 600 is configured to activate and/or deactivate cardio fitness notifications in response to user input. In some embodiments, computer system 600 displays cardio fitness user interface 628 based on a determination that cardio fitness notifications are activated (e.g., activated at a time at which computer system 600 initiates the process to add the pregnancy event to the account corresponding to computer system 600). In some embodiments, computer system 600 displays cardio fitness user interface 628 regardless of whether cardio fitness notifications are activated or deactivated.

At FIG. 6M, cardio fitness user interface 628 includes information region 628a, deactivation user interface object 628b, and skip user interface object 628c. Information region 628a includes text, images, icons, and/or graphical user interface objects that provide the user of computer system 600 with information about cardio fitness notifications and/or information about why cardio fitness notifications may not be helpful to the user while the user is pregnant. For instance, at FIG. 6M, information region 628a includes text indicating that the cardio fitness of the user may decrease during pregnancy, and thus, the user may consider deactivating cardio fitness notifications during the pregnancy event and/or for a threshold duration (e.g., 6 weeks, 12 weeks, 16 weeks, and/or 24 weeks) after the pregnancy event. At FIG. 6M, computer system 600 detects user input 650t (e.g., a tap input, a touch input, an air gesture, a mouse click, a gaze input, and/or a speech input) corresponding to deactivation user interface object 628b. In response to detecting user input 650t, computer system 600 initiates a process for deactivating cardio fitness notifications and displays mental health user interface 630, as shown at FIG. 6N. Alternatively, at FIG. 6M, computer system 600 detects user input 650u (e.g., a tap input, a touch input, an air gesture, a mouse click, a gaze input, and/or a speech input) corresponding to skip user interface object 628c. In some embodiments, in response to detecting user input 650u, computer system 600 displays mental health user interface 630 without initiating the process for deactivating cardio fitness notifications.

At FIG. 6N, mental health user interface 630 provides one or more options for managing and/or configuring notifications related to the mental health of the user. In some embodiments, computer system 600 is configured to output notifications, prompts, alerts, and/or reminders related to a mental health questionnaire. In some embodiments, computer system 600 outputs the notifications, prompts, alerts, and/or reminders at predefined intervals of time (e.g., once a day, once a week, one every two weeks, once a month, once every six months, or once a year). In some embodiments, computer system 600 is configured to adjust the predefined intervals of time in response to user input and/or based on contextual factors (e.g., an age and/or medical history) of the user. In some embodiments, computer system 600 displays a mental health questionnaire user interface in response to one or more user inputs. In some embodiments, computer system 600 is configured to track, estimate, and/or determine a mental health of the user of computer system 600 based on inputs received while displaying the mental health questionnaire user interface. In some embodiments, computer system 600 is configured to activate and/or deactivate mental health notifications in response to user input. In some embodiments, computer system 600 displays mental health user interface 630 based on a determination that mental health notifications are deactivated (e.g., deactivated at a time at which computer system 600 initiates the process to add the pregnancy event to the account corresponding to computer system 600). In some embodiments, computer system 600 displays mental health user interface 630 regardless of whether mental health notifications are activated or deactivated.

At FIG. 6N, mental health user interface 630 includes information region 630a, activation user interface object 630b, and skip user interface object 630c. Information region 630a includes text, images, icons, and/or graphical user interface objects that provide the user of computer system 600 with information about mental health notifications, information about why mental health notifications may be beneficial to the user while the user is pregnant, and/or information about adjusting a frequency for receiving mental health notifications while the user is pregnant. For instance, at FIG. 6N, information region 630a includes text indicating that the pregnancy event can affect the mental health of the user and/or that activating the mental health notifications causes computer system 600 to output mental health notifications at predefined intervals (e.g., once a week, two times a month, or one time a month) during the pregnancy event. At FIG. 6N, computer system 600 detects user input 650v (e.g., a tap input, a touch input, an air gesture, a mouse click, a gaze input, and/or a speech input) corresponding to activation user interface object 630b. In response to detecting user input 650v, computer system 600 initiates a process for activating mental health notifications, modifies and/or adjusts the frequency for outputting mental health notifications, and displays medications user interface 632, as shown at FIG. 6O. Alternatively, at FIG. 6N, computer system 600 detects user input 650w (e.g., a tap input, a touch input, an air gesture, a mouse click, a gaze input, and/or a speech input) corresponding to skip user interface object 630c. In some embodiments, in response to detecting user input 650w, computer system 600 displays medications user interface 632 without initiating the process for activating mental health notifications and/or modifying the frequency for outputting mental health notifications.

At FIG. 6O, medications user interface 632 includes medication region 632a corresponding to medications that are associated with the account corresponding to computer system 600. For instance, in some embodiments, computer system 600 detects one or more user inputs corresponding to information about medications in which the user of the account is taking, has taken, and/or is planning to take. In some embodiments, the information about the medication includes a type of medication, a name of the medication, a dose of the medication, and/or a form of the medication (e.g., whether the medication is a pill, an injection, a powder, and/or a cream). At FIG. 6O, medication region 632a indicates that computer system 600 has received information (e.g., detected one or more user inputs) corresponding to the user of the account taking one medication (e.g., insulin lispro). At FIG. 6O, computer system 600 displays medication region 632a without displaying warnings and/or alerts related to the medication in medication region 632a. As set forth below, in some embodiments, computer system 600 determines whether medication information of the account corresponding to computer system 600 are known to and/or can affect the user during pregnancy. In some embodiments, computer system 600 outputs warnings and/or alerts based on a determination that a medication of the account corresponding to computer system 600 is known to and/or can affect the user during pregnancy.

At FIG. 6O, medications user interface 632 includes add medication user interface object 632b, which enables computer system 600 to associate additional medications with the account corresponding to computer system 600. For example, in some embodiments, the user of computer system 600 plans to take additional medications (e.g., because of the pregnancy event) and/or has not updated the medication information of the account to include all medications being taken by the user. As such, medications user interface 632 includes add medication user interface object 632b to enable the user to update the account to include the most recent and/or accurate medication information. In addition, at FIG. 6O, medications user interface 632 includes next user interface object 632c. In some embodiments, in response to detecting user input corresponding to next user interface object 632c, computer system 600 displays completion user interface 634, as shown at FIG. 6S, without updating the medication information of the account corresponding to computer system 600.

At FIG. 6O, computer system 600 detects user input 650x (e.g., a tap input, a touch input, an air gesture, a mouse click, a gaze input, and/or a speech input) corresponding to add medication user interface object 632b. In response to detecting user input 650x, computer system 600 displays add medication user interface 636, as shown at FIG. 6P.

At FIG. 6P, add medication user interface 636 includes search user interface object 636a and camera user interface object 636b. In some embodiments, in response to detecting user input corresponding to camera user interface object 636b, computer system 600 launches and/or activates a camera in communication with computer system 600 so that the user can take a picture of a medication. In some embodiments, computer system 600 is configured to determine medication information based on an image captured via the camera that is in communication with computer system 600. At FIG. 6P, computer system 600 detects one or more user inputs 650y (e.g., a tap input, a touch input, an air gesture, a mouse click, a gaze input, and/or a speech input) corresponding to search user interface object 636a. In some embodiments, one or more user inputs 650y include a first input corresponding to search user interface object 636a, one or more second inputs corresponding to a keyboard displayed by computer system 600 for inputting alphanumeric characters in search user interface object 636a, and/or one or more third inputs corresponding to selection of a respective medication to add to the medication information of the account corresponding to computer system 600. After detecting one or more user inputs 650y, computer system 600 displays (e.g., re-displays) medications user interface 632, as shown at FIG. 6Q.

At FIG. 6Q, computer system 600 updates medication user interface 632 to include medication platter 632d corresponding to a new medication added to the account by computer system 600 in response to detecting one or more user inputs 650y. At FIG. 6Q, computer system 600 does not display medication platter 632d in medication region 632a. Instead, computer system 600 visually distinguishes medication platter 632d from medication region 632a to provide visual feedback to the user that the medication corresponding to medication platter 632d can affect the pregnancy event. For instance, as set forth above, in some embodiments, computer system 600 determines whether medications of the account corresponding to computer system 600 are known to and/or can affect pregnancy. At FIG. 6Q, computer system 600 determines that the medication corresponding to medication platter 632d is known to and/or can affect pregnancy. For instance, medication platter 632d corresponds to a blood thinner, which is a medication known to affect pregnancies. As such, computer system displays medication platter 632d with alert indicator 632e providing further visual feedback to the user that the medication associated with medication platter 632d can affect the pregnancy event. In some embodiments, computer system 600 determines that the medication added to the account in response to one or more user inputs 650y is not known to and/or does not affect pregnancy. In some embodiments, computer system 600 displays information about the medication added to the account in response to one or more user inputs 650y to medication region 632a based on a determination that the medication is not known to and/or does not affect pregnancy.

At FIG. 6Q, computer system 600 detects user input 650z (e.g., a tap input, a touch input, an air gesture, a mouse click, a gaze input, and/or a speech input) corresponding to medication platter 632d. In response to detecting user input 650z, computer system 600 displays medication alert user interface 638, as shown at FIG. 6R. Alternatively, computer system 600 detects user input 650aa (e.g., a tap input, a touch input, an air gesture, a mouse click, a gaze input, and/or a speech input) corresponding to next user interface object 632c of medication user interface 632. In some embodiments, in response to detecting user input 650aa, computer system 600 displays completion user interface 634, as shown at FIG. 6S, without displaying medication alert user interface 638.

At FIG. 6R, computer system 600 displays medication alert user interface 638. Medication alert user interface 638 includes medication region 638a and information region 638b. Medication region 638a includes details, such as a name, a type of medication, a dose, and/or a form of the medication corresponding to medication platter 632d. For instance, at FIG. 6P, medication region 638a includes information about the name of the blood thinner (e.g., “Warfarin”), that the blood thinner is a capsule (e.g., pill), the dose of the blood thinner (e.g., 100 mg), and/or other medications that are taken with the blood thinner (e.g., 100 mg of Ibuprofen). In addition, information region 638b includes additional information about the potential effects of taking the medication during pregnancy. For instance, information region 638b includes text indicating that blood thinners potentially cause dangerous and/or fatal side effects for pregnant users. In addition, information region 638b includes text prompting the user to consult with their doctor before continuing to take the medication corresponding to medication platter 632d.

At FIG. 6P, computer system 600 detects user input 650ab (e.g., a tap input, a touch input, an air gesture, a mouse click, a gaze input, and/or a speech input) corresponding to cancel user interface object 638c of medication alert user interface 638. In some embodiments, computer system 600 displays (e.g., re-displays) medications user interface 632 in response to detecting user input 650ab. In some embodiments, computer system 600 displays completion user interface 634 in response to detecting user input 650ab.

At FIG. 6S, computer system 600 displays completion user interface 634 indicating that the pregnancy event has been added to the account corresponding to computer system 600. Completion user interface 634 includes completion indicator 634a, indicating that the process for adding the pregnancy event to the account is complete and/or that information entered during the process can be edited, modified, and/or changed (e.g., via one or more user inputs).

At FIG. 6S, computer system 600 detects user input 650ac (e.g., a tap input, a touch input, an air gesture, a mouse click, a gaze input, and/or a speech input) corresponding to done user interface object 634b of completion user interface 634. In response to detecting user input 650ac, computer system 600 displays pregnancy room user interface 640, as shown at FIG. 6T.

At FIG. 6T, pregnancy room user interface 640 includes information and/or visual indications related to the pregnancy event added to the account corresponding to computer system 600 (and, optionally, past pregnancy events added to the account corresponding to computer system 600). For instance, at FIG. 6T, pregnancy room user interface 640 includes timeline 642, which provides a visual indication of the age of the pregnancy event relative to a start date of the pregnancy event and an estimated due date of the pregnancy event. Timeline 642 includes first portion 642a corresponding to an amount of time that has elapsed since the start date of the pregnancy event. Timeline 642 also includes second portion 642b corresponding to an amount of time from a current date (e.g., a date at which computer system 600 displays pregnancy room user interface) to the estimated due date of the pregnancy event. At FIG. 6T, the current age of the pregnancy event is 5 weeks and 1 day. Because the typical duration of a pregnancy is nine months, computer system 600 displays first portion 642a of timeline 642 as shorter and/or smaller when compared to second portion 642b. For instance, because the current age of the pregnancy event is 5 weeks and 1 day, there is more time between the current date and the estimated due date than between the start date of the pregnancy event and the current date.

At FIG. 6T, pregnancy room user interface 640 includes current pregnancy platter 640a and past pregnancy platter 640b. Current pregnancy platter 640a corresponds to the pregnancy event added to the account described above with reference to FIGS. 6A-6S. In some embodiments, in response to detecting user input corresponding to current pregnancy platter 640a, computer system 600 displays one or more user interfaces that allow the user of computer system 600 to edit, modify, update, and/or change information related to the current pregnancy event. Past pregnancy platter 640b corresponds to a pregnancy event that has ended (e.g., ended Mar. 9, 2024). In some embodiments, computer system 600 stores and/or records information related to pregnancy events, such that the information related to past pregnancies is accessible via pregnancy room user interface 640. In some embodiments, when the account corresponding to computer system 600 does not include information related to past pregnancies, computer system 600 forgoes display of past pregnancy platter 640b on pregnancy room user interface 640. In some embodiments, in response to user input corresponding to past pregnancy platter 640b, computer system 600 displays one or more user interfaces that includes text, images, icons, timelines, and/or graphical user interface objects that provide information about a past pregnancy event of the account corresponding to computer system 600.

In some embodiments, computer system 600 prompts a user to remove the pregnancy designation and/or pregnancy event from the account corresponding to computer system 600. For example, when computer system 600 includes information related to an estimated due date of the pregnancy event, computer system 600 outputs a prompt to remove the pregnancy designation and/or pregnancy event from the account within a threshold duration of the estimated due date. In some embodiments, the threshold duration is within 1 day, within 1 week, within 2 weeks, or within 1 month of the estimated due date. In some embodiments, the threshold duration includes dates before and after the estimated due date. In some embodiments, the threshold duration includes dates after the estimate due date and does not include dates before the estimate due date.

At FIG. 6U, computer system 600 displays prompt 644 based on a determination that the current date (e.g., a date at which computer system 600 displays prompt 644) is within the threshold duration of the estimated due date of the pregnancy event. At FIG. 6U, prompt 644 includes information 644a that prompt 644 is related to removing the pregnancy designation and/or pregnancy event from a medical identification profile of the account corresponding to computer system 600. In some embodiments, the medical identification profile includes a sub-set of information of the account corresponding to computer system 600. In some embodiments, the medical identification profile includes information that is accessible and/or displayed by computer system 600 without requiring an authorization input, such as a biometric authentication input and/or a password. In some embodiments, computer system 600 adds a pregnancy designation to the medical identification profile of the account as a portion of the process for adding the pregnancy event to the account. In some embodiments, computer system 600 displays a separate user interface during the process for adding the pregnancy event to the account that prompts the user of computer system 600 to confirm whether to add the pregnancy designation to the medical identification profile. At FIG. 6U, prompt 644 includes skip user interface object 644b that allows the user of computer system 600 to dismiss prompt 644 without removing the pregnancy designation from the medical identification profile (and, optionally, without removing the pregnancy event from the account corresponding to computer system 600). In some embodiments, in response to detecting user input corresponding to skip user interface object 644b, computer system 600 removes display of prompt 644 and displays a different user interface, such as health user interface 604 and/or a user interface displayed prior to computer system 600 displaying prompt 644.

At FIG. 6U, computer system 600 detects user input 650ad (e.g., a tap input, a touch input, an air gesture, a mouse click, a gaze input, and/or a speech input) corresponding to remove user interface object 644c of prompt 644. In response to detecting user input 650ad, computer system 600 removes the pregnancy designation from the medical identification profile of the account. In some embodiments, computer system 600 also removes the pregnancy event and/or pregnancy designation from the account corresponding to computer system 600 in response to detecting user input 650ad. After and/or in response to detecting user input 650ad, computer system 600 displays after pregnancy user interface 646, as shown at FIG. 6V.

At FIG. 6V, after pregnancy user interface 646 provides options for adding one or more factors to a cycle tracking function of computer system 600, such as the cycle tracking function corresponding to cycle tracking user interface 606 described above with reference to FIG. 6B. At FIG. 6V, after pregnancy user interface 646 includes selectable factor options 646a-646c, which correspond to symptoms and/or circumstances that a person frequently experiences after pregnancy that can affect their menstrual cycle. For instance, selectable factor option 646a corresponds to lactation, selectable factor option 646b corresponds to contraceptives, and selectable factor 646c corresponds to a pregnancy event (e.g., a new pregnancy event). As such, computer system 600 provides the user with options to designate one or more factors that they may be experiencing without requiring the user to navigate to cycle tracking user interface 606. At FIG. 6V, after pregnancy user interface 646 includes no factors selectable option 646d in addition to selectable factor options 646a-646c. No factors selectable option 646d corresponds to an option related to the user of computer system 600 not experiencing any common symptoms and/or circumstances that affect their menstrual cycle after pregnancy. Thus, the user of computer system 600 can determine whether or not to add factors to the cycle tracking function of computer system 600 after removing the pregnancy designation from the medical identification profile and/or removing the pregnancy event from the account corresponding to computer system 600.

At FIG. 6V, computer system 600 detects user input 650ae (e.g., a tap input, a touch input, an air gesture, a mouse click, a gaze input, and/or a speech input) corresponding to selectable factor option 646a. In response to detecting user input 650ae, computer system 600 displays selection indicator 646e indicating that selectable factor option 646a is currently selected. After detecting user input 650ae, computer system 600 detects user input 650af (e.g., a tap input, a touch input, an air gesture, a mouse click, a gaze input, and/or a speech input) corresponding to done user interface object 646f. In response to detecting user input 650af, computer system 600 displays confirmation user interface 648, as shown at FIG. 6W.

At FIG. 6W, confirmation user interface 648 includes information 648a indicating that information related to the pregnancy event has been updated. In some embodiments, in response to detecting user input corresponding to done user interface 648b, computer system 600 displays health user interface 604 and/or cycle tracking user interface 606.

FIG. 7 is a flow diagram illustrating a method for managing notifications related to a pregnancy event using a computer system in accordance with some embodiments. Method 700 is performed at a computer system (e.g., 100, 300, 500, and/or 600) (e.g., a smart phone, a smart watch, a tablet computer, a laptop computer, a desktop computer, a wearable device, and/or a head-mounted device) that is in communication with one or more display generation components (e.g., 602) (e.g., one or more displays, touch-screen displays, monitors, holographic display systems, and/or head-mounted display systems) and one or more input devices (e.g., 602) (e.g., a touch-sensitive surface (e.g., a touch-sensitive display); a mouse; a keyboard; a remote control; a visual input device (e.g., one or more cameras such as, e.g., an infrared camera, a depth camera, a visible light camera, and/or a gaze tracking camera); an audio input device (e.g., a microphone); a biometric sensor (e.g., a fingerprint sensor, a face identification sensor, a gaze tracking sensor, and/or an iris identification sensor); and/or one or more mechanical input devices (e.g., a depressible input mechanism; a button; a rotatable input mechanism; a crown; and/or a dial)). Some operations in method 700 are, optionally, combined, the orders of some operations are, optionally, changed, and some operations are, optionally, omitted.

As described below, method 700 provides an intuitive way for managing notifications related to a pregnancy event. The method reduces the cognitive burden on a user for managing notifications related to a pregnancy event, thereby creating a more efficient human-machine interface. For battery-operated computing devices, enabling a user to manage notifications related to a pregnancy event faster and more efficiently conserves power and increases the time between battery charges.

The computer system (e.g., 100, 300, 500, and/or 600) detects (702), via the one or more input devices (e.g., 602), one or more user inputs (e.g., 650a-6500 and/or 650ac) corresponding to a request (e.g., one or more tap gestures, swipe gestures, touch gestures, air gestures, button presses, and/or voice commands) to add a pregnancy event (e.g., a designation that a user of the computer system is pregnant and/or information related to a pregnancy of the user of the computer system) to health information (e.g., a health profile and/or a health application that corresponds to and/or includes received and/or detected health and/or activity information of a user of the computer system) of an account (e.g., an account and/or user profile of a user of the computer system that is accessible by the computer system) of the computer system (e.g., 100, 300, 500, and/or 600).

In response to detecting the one or more user inputs (e.g., 650a-6500 and/or 650ac) corresponding to the request to add the pregnancy event to the health information of the account of the computer system (e.g., 100, 300, 500, and/or 600), the computer system (e.g., 100, 300, 500, and/or 600) displays (704), via the one or more display generation components (e.g., 602), a pregnancy user interface (e.g., 614 and/or 622) (e.g., a user interface that includes text, objects, icons, images, and/or one or more selectable user interface objects corresponding to pregnancy information, entering and/or providing pregnancy information, and/or estimating an age of a pregnancy) that includes a pregnancy estimation user interface object (e.g., 614a, 614b, 614e, 614f, and/or 622a-622d) (e.g., a button, selectable icon, affordance, and/or user-interactive user interface element for receiving, selecting, and/or entering information related to an age, duration, and/or due date of a pregnancy).

The computer system (e.g., 100, 300, 500, and/or 600) detects (706), via the one or more input devices (e.g., 602), one or more user inputs (e.g., 650j-650n) (e.g., one or more tap gestures, swipe gestures, touch gestures, air gestures, button presses, and/or voice commands) corresponding to the pregnancy estimation user interface object (e.g., 614a, 614b, 614e, 614f, and/or 622a-622d).

In response to detecting the one or more user inputs (e.g., 650j-650n) corresponding to the pregnancy estimation user interface object (e.g., 614a, 614b, 614e, 614f, and/or 622a-622d), the computer system (e.g., 100, 300, 500, and/or 600) receives (708) information about an age of the pregnancy event (e.g., information shown at FIG. 6I) (e.g., information about a time and/or time range of a last menstrual period of a user of the computer system, an approximate and/or estimated due date of the pregnancy of the user of the computer system, an approximate and/or estimated gestational age of the pregnancy of the user of the computer system, an embryo transfer date of the pregnancy of the user of the computer system, and/or an approximate and/or estimated conception date of the pregnancy of the user of the computer system).

After (e.g., in response to) receiving the information (e.g., information shown at FIG. 6I) about the age of the pregnancy event, the computer system (e.g., 100, 300, 500, and/or 600) displays (710), via the one or more display generation components (e.g., 602), one or more user interfaces (e.g., 624, 626, 628, and/or 630) (e.g., one or more user interfaces that are displayed sequentially or concurrently) corresponding to options to configure (e.g., enabling or disabling notifications, adjusting and/or modifying a trigger and/or context for outputting notifications, and/or adjusting and/or modifying a frequency and/or timing of outputting notifications) respective notifications (e.g., notifications corresponding user interfaces 624, 626, 628, and/or 630) (e.g., user interfaces, text, images, icons, audio, alerts, and/or graphical user interface objects that are output by the computer system in response to receiving information, in response to detecting an event, and/or in response to a contextual trigger) related to the pregnancy event (e.g., notifications that are relevant to the pregnancy of the user of the computer system, such as notifications about health, wellness, and/or fitness of the user of the computer system, notifications about activity performed by the user that may affect and/or provide relevant information about the pregnancy of the user, and/or notifications that remind and/or guide the user of the computer system to perform an action related to the pregnancy event).

Displaying the one or more user interfaces corresponding to options to configure respective notifications related to the pregnancy event after receiving the information about the age of the pregnancy event allows the user of the computer system to customize notifications that are output by the computer system without requiring the user to navigate to an additional user interface, thereby reducing the number of inputs needed to perform an operation and/or performing an operation when a set of conditions has been met without requiring further user input.

In some embodiments, the information (e.g., information shown at FIG. 6I) about the age of the pregnancy event includes one or more of: a last menstrual period (e.g., 622a) (e.g., a date range of a most recent menstrual period of the user corresponding to the account of the computer system, an end date of the most recent menstrual period of the user corresponding to the account of the computer system, and/or a start date of the most recent menstrual period of the user corresponding to the account of the computer system), an approximate due date of the pregnancy event (e.g., 622b) (e.g., an estimated, predicted, and/or projected date at which the user corresponding to the account of the computer system will and/or is likely to give birth, such as a date provided to the user by a physician and/or doctor and/or a date estimated by the user), an approximate gestational age of the pregnancy event (e.g., 622c) (e.g., an estimated, predicted, and/or projected date at which the pregnancy of the user corresponding to the account of the computer system started and/or was conceived, such as a date provided to the user by a physician and/or doctor and/or a date estimated by the user), and a date of an embryo transfer (e.g., 622d) (e.g., an estimated, predicted, projected, and/or exact date at which an embryo was transferred to the user corresponding to the account of the computer system). The information about the age of the pregnancy event including a date of a last menstrual period, an approximate due date of the pregnancy event, an approximate gestational age of the pregnancy event, and/or a date of an embryo transfer provides the user of the computer system with different options for estimating the age of the pregnancy event without requiring the user to perform additional user inputs, thereby reducing the number of inputs needed to perform an operation.

In some embodiments, in response to receiving the information (e.g., information shown at FIG. 6I) about the age of the pregnancy event and prior to displaying the one or more user interfaces (e.g., 624, 626, 628, and/or 630) corresponding to options to configure respective notifications (e.g., notifications corresponding to user interfaces 624, 626, 628, and/or 630) related to the pregnancy event, the computer system (e.g., 100, 300, 500, and/or 600) displays, via the one or more display generation components (e.g., 602), estimated pregnancy information (e.g., information shown at 614b, 614e, and/or 614f shown at FIG. 6I) (e.g., estimated, predicted, and/or projected age of the pregnancy and/or due date of the pregnancy) that is based on (e.g., determined and/or calculated at least partially based on) the information (e.g., information shown at FIG. 6I) about the age of the pregnancy event. Displaying the estimated pregnancy information that is based on the information about the age of the pregnancy event allows a user to view the estimated pregnancy information without requiring the user to navigate to a different user interface, thereby reducing the number of inputs needed to perform an operation and/or performing an operation when a set of conditions has been met without requiring further user input.

In some embodiments, the estimated pregnancy information includes an estimated pregnancy age (e.g., 614e) (e.g., an approximate, predicted, and/or projected duration and/or amount of time at which the user corresponding to the account of the computer system has been pregnant and/or an approximate, predicted, and/or projected date at which the pregnancy of the user began and/or was conceived). Displaying the estimated pregnancy age allows a user to view the estimated pregnancy age without requiring the user to navigate to a different user interface, thereby reducing the number of inputs needed to perform an operation and/or performing an operation when a set of conditions has been met without requiring further user input.

In some embodiments, the estimated pregnancy information includes an estimated due date (e.g., 614b) (e.g., an approximate, predicted, and/or projected date at which the user corresponding to the account of the computer system will and/or is likely to give birth). Displaying the estimated due date allows a user to view the estimated due date without requiring the user to navigate to a different user interface, thereby reducing the number of inputs needed to perform an operation and/or performing an operation when a set of conditions has been met without requiring further user input.

In some embodiments, the one or more user interfaces (e.g., 624, 626, 628, and/or 630) corresponding to options to configure respective notifications (e.g., notifications corresponding to user interfaces 624, 626, 628, and/or 630) related to the pregnancy event include a walking steadiness user interface (e.g., 624) (e.g., a user interface that allows a user to configure notifications related to a determined, estimated, and/or approximate stability and/or a fall risk of a user while walking, which may be based on walking speed, step length, double support time, and/or walking asymmetry data) corresponding to options to configure walking steadiness notifications (e.g., notifications corresponding to walking steadiness user interface 624) (e.g., notifications, alerts, user interfaces, audio, and/or tactile feedback that is output by the computer system when the computer system determines that the walking steadiness of a user corresponding to the account of the computer system is below one or more threshold values). The one or more user interfaces corresponding to options to configure respective notifications related to the pregnancy event including a walking steadiness user interface corresponding to options to configure walking steadiness notifications allows a user to determine whether to adjust and/or modify walking steadiness notifications without requiring the user to navigate to another user interface, thereby reducing the number of inputs needed to perform an operation and/or performing an operation when a set of conditions has been met without requiring further user input.

In some embodiments, the one or more user interfaces (e.g., 624, 626, 628, and/or 630) corresponding to options to configure respective notifications (e.g., notifications corresponding to user interfaces 624, 626, 628, and/or 630) related to the pregnancy event include a cardio fitness user interface (e.g., 628) (e.g., a user interface that allows a user to configure notifications and/or data collection related to a determined, estimated, and/or approximate maximum amount of oxygen in which a user corresponding to the account of the computer system can consume during exercise, a determined, estimated, and/or approximate cardiorespiratory fitness level of the user, and/or a determined, estimated, and/or approximate VO2 max of the user) corresponding to options to configure cardio fitness notifications (e.g., notifications corresponding to user interface 628) (e.g., notifications, alerts, user interfaces, audio, and/or tactile feedback that is output by the computer system when the computer system determines that the cardio fitness of the user is below one or more threshold values and/or notifications, alerts, user interfaces, audio, and/or tactile feedback that is output by the computer system and includes an indication of a value of cardio fitness of the user). The one or more user interfaces corresponding to options to configure respective notifications related to the pregnancy event including a cardio fitness user interface corresponding to options to configure cardio fitness notifications allows a user to determine whether to adjust and/or modify cardio fitness notifications without requiring the user to navigate to another user interface, thereby reducing the number of inputs needed to perform an operation and/or performing an operation when a set of conditions has been met without requiring further user input.

In some embodiments, the one or more user interfaces (e.g., 624, 626, 628, and/or 630) corresponding to options to configure respective notifications (e.g., notifications corresponding to user interfaces 624, 626, 628, and/or 630) related to the pregnancy event include a mental health user interface (e.g., 630) (e.g., a user interface that allows a user to configure notifications related to a questionnaire that provides a determined, estimated, and/or approximate assessment of a mental health of a user corresponding to the account of the computer system and/or a user interface that allows a user to configure notifications related to prompting the user to answer questions related to their mental health to log various emotions and/or feelings in which the user is experiencing) corresponding to options to configure mental health notifications (e.g., notifications corresponding to user interface 630) (e.g., notifications, alerts, user interfaces, audio, and/or tactile feedback that is output by the computer system at respective intervals of time to prompt the user to provide information about their mental health). The one or more user interfaces corresponding to options to configure respective notifications related to the pregnancy event including a mental health user interface corresponding to options to configure mental health notifications allows a user to determine whether to adjust and/or modify mental health notifications without requiring the user to navigate to another user interface, thereby reducing the number of inputs needed to perform an operation and/or performing an operation when a set of conditions has been met without requiring further user input.

In some embodiments, after displaying the one or more user interfaces (e.g., 624, 626, 628, and/or 630) corresponding to options to configure respective notifications (e.g., notifications corresponding to user interfaces 624, 626, 628, and/or 630) related to the pregnancy event, the computer system (e.g., 100, 300, 500, and/or 600) displays (e.g., in response to receiving one or more user inputs while and/or after displaying the one or more user interfaces corresponding to the options to configure respective notifications related to the pregnancy event), via the one or more display generation components (e.g., 602), a medication user interface (e.g., 632 and/or 636) (e.g., a user interface that includes text, images, icons, and/or graphical user interface objects that provide information about medications corresponding to the account of the computer system, such as medications that the user corresponding to the account of the computer system designated and/or indicated as having taken and/or currently taking and/or a user interface that provides one or more options for adding medications to the health information of the account of the computer system). Displaying the medication user interface after displaying the one or more user interfaces corresponding to options to configure respective notifications related to the pregnancy event allows a user to view, remove, and/or add medications to the health information of the account of the computer system without requiring the user to navigate to a different user interface, thereby reducing the number of inputs needed to perform an operation and/or performing an operation when a set of conditions has been met without requiring further user input.

In some embodiments, while displaying the medication user interface (e.g., 632 and/or 636), the computer system (e.g., 100, 300, 500, and/or 600) detects, via the one or more input devices (e.g., 602), user input (e.g., 650x and/or 650y) corresponding to a request to (e.g., a tap gestures, a swipe gesture, a touch gesture, an air gesture, a button press, and/or a voice command) add a medication to the health information of the account of the computer system (e.g., 100, 300, 500, and/or 600) (e.g., designate and/or indicate that the user corresponding to the account of the computer system has taken, is currently taking, and/or is planning to take a particular and/or respective medication, such as taking and/or planning to take the particular and/or respective medication during pregnancy). In response to detecting the user input (e.g., 650x and/or 650y) corresponding to the request to add the medication to the health information of the account of the computer system (e.g., 100, 300, 500, and/or 600), the computer system (e.g., 100, 300, 500, and/or 600) displays, via the one or more display generation components (e.g., 602), information about the medication (e.g., 632d) (e.g., a name of the medication, a dose of the medication, a description of the medication, a type of the medication, a state of the medication (e.g., a pill, an injection, and/or a liquid), whether the medication is known to react with other medications, and/or whether the medication is known to affect pregnancy). Displaying information about the medication in response to detecting the user input corresponding to the request to add the medication to the health information of the account of the computer system allows a user to view the information about the medication without requiring the user to navigate to a different user interface, thereby reducing the number of inputs needed to perform an operation and/or performing an operation when a set of conditions has been met without requiring further user input.

In some embodiments, while displaying the medication user interface (e.g., 632 and/or 636), the computer system (e.g., 100, 300, 500, and/or 600) detects, via the one or more input devices (e.g., 602), user input (e.g., 650x and/or 650y) corresponding to a request to (e.g., a tap gestures, a swipe gesture, a touch gesture, an air gesture, a button press, and/or a voice command) add a second medication (e.g., the medication corresponding to medication platter 632d) (e.g., the same medication as the medication and/or a medication that is different from the medication) to the health information of the account of the computer system (e.g., 100, 300, 500, and/or 600) (e.g., designate and/or indicate that the user corresponding to the account of the computer system has taken, is currently taking, and/or is planning to take a particular and/or respective medication, such as taking and/or planning to take the particular and/or respective medication during pregnancy). In response to detecting the user input (e.g., 650x and/or 650y) corresponding to the request to add the second medication (e.g., the medication corresponding to medication platter 632d) to the health information of the account of the computer system (e.g., 100, 300, 500, and/or 600) and in accordance with a determination that the second medication (e.g., the medication corresponding to medication platter 632d) satisfies a set of criteria (e.g., the second medication is known to affect, cause complications, and/or otherwise result in adverse effects during pregnancy and/or the second medication is known to react with other medications in an adverse manner), the computer system (e.g., 100, 300, 500, and/or 600) outputs an alert (e.g., 632e and/or 638b) (e.g., text, a user interface, one or more user interface objects, audio, and/or tactile feedback) corresponding to the second medication (e.g., the medication corresponding to medication platter 632d) (e.g., the alert provides information that the second medication can affect the pregnancy event). In some embodiments, in accordance with a determination that the second medication (e.g., the medication corresponding to medication platter 632d) does not satisfy the set of criteria (e.g., the second medication is not known to affect, cause complications with, and/or otherwise result in adverse effects during pregnancy and/or the second medication is not known to react with other medications in an adverse manner), the computer system (e.g., 100, 300, 500, and/or 600) forgoes output of the alert (e.g., 632e and/or 638b) corresponding to the second medication (e.g., the medication corresponding to medication platter 632d). Outputting the alert corresponding to the second medication in response to detecting the user input corresponding to the request to add the second medication to the health information of the account of the computer system and in accordance with a determination that the second medication satisfies the set of criteria allows a user to view information about the second medication without requiring the user to navigate to a different user interface, thereby reducing the number of inputs needed to perform an operation and/or performing an operation when a set of conditions has been met without requiring further user input.

In some embodiments, after displaying the one or more user interfaces (e.g., 624, 626, 628, and/or 630) corresponding to options to configure respective notifications (e.g., notifications corresponding to user interfaces 624, 626, 628, and/or 630) related to the pregnancy event, the computer system (e.g., 100, 300, 500, and/or 600) displays, via the one or more display generation components (e.g., 602), a health settings user interface (e.g., 620, 624, 626, 628, 646, and/or 1012) (e.g., a user interface that includes one or more user interface objects, selectable options, and/or user interactive elements that allow a user to adjust, modify, change, and/or set various targets, goals, and/or thresholds related to health information and/or activity information), wherein the health settings user interface (e.g., 620, 624, 626, 628, 646, and/or 1012) includes a user interface object (e.g., 620b, 624, 626b, 628b, 640a, and/or 1012b) (e.g., a button, an affordance, a graphical user interface element, a user selectable icon, a menu, and/or a region of a display generation component of the one or more display generation components) corresponding to an option to change an existing health setting (e.g., adjust and/or modify an existing health setting from a first state, such as a first target, goal, and/or threshold, to a second state, such as a second target, goal, and/or threshold). While displaying the health settings user interface (e.g., 620, 624, 626, 628, 646, and/or 1012), the computer system (e.g., 100, 300, 500, and/or 600) detects, via the one or more input devices (e.g., 602), user input (e.g., 6500, 650p, 650r, and/or 650t) (e.g., a tap gestures, a swipe gesture, a touch gesture, an air gesture, a button press, and/or a voice command) corresponding to (e.g., selecting and/or interacting with) the health settings user interface (e.g., 620, 624, 626, 628, 646, and/or 1012). In response to detecting the user input (e.g., 6500, 650p, 650r, and/or 650t) corresponding to the health settings user interface (e.g., 620, 624, 626, 628, 646, and/or 1012) and in accordance with a determination that the user input (e.g., 6500, 650p, 650r, and/or 650t) corresponds to the user interface object (e.g., 620b, 624b, 626b, 628b, 640a, and/or 1012b) corresponding to the option to change the existing health setting, the computer system (e.g., 100, 300, 500, and/or 600) adjusts the existing health setting (e.g., changing and/or modifying the existing health setting from a first state, such as a first target, goal, and/or threshold, to a second state, such as a second target, goal, and/or threshold). In some embodiments, in accordance with a determination that the user input does not correspond to the user interface object corresponding to the option to change the existing health setting, the computer system (e.g., 100, 300, 500, and/or 600) forgoes adjusting (e.g., maintains a value of and/or a state of) the existing health setting. Adjusting the health setting in response to detecting the user input corresponding to the health settings user interface and in accordance with a determination that the user input corresponds to the user interface object allows a user to change the existing health setting without requiring the user to navigate to a different user interface, thereby reducing the number of inputs needed to perform an operation.

In some embodiments, the user interface object (e.g., 620b, 624b, 626b, 628b, 640a, and/or 1012b) corresponding to the option to change the existing health setting corresponds to an option to change (e.g., adjust and/or modify a threshold heart rate from a first value, such as an existing threshold value, to a second value) a threshold heart rate (e.g., 626b) (e.g., a heart rate value in which heart rate data is compared, such that the computer system is configured to output alerts and/or notifications when received heart rate data of the user corresponding to the account of the computer system is above or below the heart rate value and/or threshold heart rate). The user interface object corresponding to the option to change the existing health setting corresponding to an option to change a threshold heart rate allows a user to change the threshold heart rate without requiring the user to navigate to a different user interface, thereby reducing the number of inputs needed to perform an operation.

In some embodiments, the user interface object (e.g., 620b, 624b, 626b, 628b, 640a, and/or 1012b) corresponding to the option to change the existing health setting corresponds to an option to change (e.g., adjust and/or modify a walking steadiness feature from a first state, such as an active state or an inactive state, to a second state) a status of a walking steadiness feature (e.g., 624b) (e.g., a feature that tracks, estimates, determines, and/or causes the computer system to output alerts and/or notifications related to a stability and/or a fall risk of a user while walking, which may be based on walking speed, step length, double support time, and/or walking asymmetry data). The user interface object corresponding to the option to change the existing health setting corresponding to an option to change a status of a walking steadiness feature allows a user to change the status of the walking steadiness feature without requiring the user to navigate to a different user interface, thereby reducing the number of inputs needed to perform an operation.

In some embodiments, the user interface object (e.g., 620b, 624b, 626b, 628b, 640a, and/or 1012b) corresponding to the option to change the existing health setting corresponds to an option to change (e.g., adjust and/or modify a cardio fitness feature from a first state, such as an active state or an inactive state, to a second state) a status of a cardio fitness feature (e.g., 628b) (e.g., a feature that tracks, estimates, determines, and/or causes the computer system to output alerts and/or notifications related to a maximum amount of oxygen in which a user corresponding to the account of the computer system can consume during exercise, a cardiorespiratory fitness level of the user, and/or a VO2 max of the user). The user interface object corresponding to the option to change the existing health setting corresponding to an option to change a status of a cardio fitness feature allows a user to change the status of the cardio fitness feature without requiring the user to navigate to a different user interface, thereby reducing the number of inputs needed to perform an operation.

In some embodiments, the user interface object (e.g., 620b, 624b, 626b, 628b, 640a, and/or 1012b) corresponding to the option to change the existing health setting corresponds to an option (e.g., a user interface object, a selectable user interface element, and/or an affordance) to add a pregnancy designation (e.g., information and/or an indication that the user corresponding to the account of the computer system is currently pregnant) to a medical profile (e.g., 620b and/or 634b) (e.g., a medical identification profile, such as an emergency medical identification profile that can be accessed while the computer system is in a locked state without providing authentication information to cause the computer system to transition to an unlocked state) of the account of the computer system (e.g., 100, 300, 500, and/or 600). The user interface object corresponding to the option to change the existing health setting corresponding to an option to add a pregnancy designation to a medical profile of the account of the computer system allows a user to add the pregnancy designation to the medical profile without requiring the user to navigate to a different user interface, thereby reducing the number of inputs needed to perform an operation.

In some embodiments, after displaying the one or more user interfaces (e.g., 624, 626, 628, and/or 630) corresponding to options to configure respective notifications (e.g., notifications corresponding to user interfaces 624, 626, 628, and/or 630) related to the pregnancy event, the computer system (e.g., 100, 300, 500, and/or 600) adds (e.g., including information and/or storing a designation that the user corresponding to the account of the computer system is pregnant and/or was pregnant) the pregnancy event to the health information of the account of the computer system (e.g., 100, 300, 500, and/or 600). In response to detecting that a current date (e.g., a date, such as a day and time, at which the computer system is currently operating) is within a threshold duration (e.g., within one day, within one week, or within two weeks) of an estimated due date of the pregnancy event (e.g., 614b) (e.g., an approximate, predicted, and/or projected date at which the user corresponding to the account of the computer system will and/or is likely to give birth), the computer system (e.g., 100, 300, 500, and/or 600) displays, via the one or more display generation components (e.g., 602), a pregnancy completion user interface (e.g., 644, 646, and/or 648) (e.g., a user interface that includes text, one or more icons, one or more user interface objects, one or more selectable user interface elements, and/or one or more images indicating that the pregnancy is likely to have been completed and/or will complete soon and/or providing one or more options to stop and/or cease a tracking feature of the pregnancy event), wherein the pregnancy completion user interface (e.g., 644, 646, and/or 648) corresponds to an option (e.g., one or more selectable user interface objects) to remove (e.g., stop a tracking feature for the pregnancy event and/or remove a designation that the user corresponding to the account of the computer system is pregnant) the pregnancy event from the health information of the account of the computer system (e.g., 100, 300, 500, and/or 600). Displaying the pregnancy completion user interface in response to detecting that the current date is within a threshold duration of an estimated due date of the pregnancy event allows the user of the computer system to remove the pregnancy event from the health information of the account of the computer system without requiring the user to provide additional user input and/or without requiring the user to navigate to a different user interface, thereby performing an operation when a set of conditions has been met without requiring further user input.

In some embodiments, the pregnancy completion user interface (e.g., 644, 646, and/or 648) includes a user interface object (e.g., 646a) corresponding to an option to initiate a lactation tracking feature (e.g., feature in which the computer system is configured to receive and/or track information about a lactation schedule and/or lactation events of the user corresponding to the account of the computer system, such as via user input, and/or a feature in which the computer system outputs one or more prompts to remind the user corresponding to the account of the computer system to provide information about lactation and/or a lactation schedule of the user). The pregnancy completion user interface including a user interface object corresponding to an option to initiate a lactation tracking feature allows a user to initiate the lactation tracking feature without requiring the user to provide additional user input and/or without requiring the user to navigate to a different user interface, thereby reducing the number of inputs needed to perform an operation and/or performing an operation when a set of conditions has been met without requiring further user input.

In some embodiments, the pregnancy completion user interface (e.g., 644, 646, and/or 648) includes a second user interface object (e.g., 644c) corresponding to an option to remove a pregnancy designation from a medical profile of the account of the computer system (e.g., 100, 300, 500, and/or 600) (e.g., remove information and/or an indication that the user corresponding to the account of the computer system is currently pregnant from a medical identification profile, such as an emergency medical identification profile that can be accessed while the computer system is in a locked state without providing authentication information to cause the computer system to transition to an unlocked state). The pregnancy completion user interface including a second user interface object corresponding to an option to remove a pregnancy designation from a medical profile of the account of the computer system allows a user to remove the pregnancy designation from the medical profile without requiring the user to provide additional user input and/or without requiring the user to navigate to a different user interface, thereby reducing the number of inputs needed to perform an operation and/or performing an operation when a set of conditions has been met without requiring further user input.

Note that details of the processes described above with respect to method 700 (e.g., FIG. 7) are also applicable in an analogous manner to the methods described below. For example, methods 900 and 1100 optionally include one or more of the characteristics of the various methods described above with reference to method 700. For example, the computer system of method 700 can be used to add a pregnancy designation to an account of the computer systems of methods 900 and 1100. For brevity, these details are not repeated below.

FIGS. 8A-8L illustrate exemplary user interfaces for tracking a pregnancy event, in accordance with some embodiments. The user interfaces in these figures are used to illustrate the processes described below, including the processes in FIG. 9.

FIG. 8A illustrates computer system 600 displaying cycle tracking user interface 606, which includes information related to a menstrual cycle of a user corresponding to an account of computer system 600. As set forth above, in some embodiments, the user of computer system 600 establishes an account that is logged into and/or otherwise accessible by computer system 600. Computer system 600 is configured to track, store, and/or otherwise receive information corresponding to health information of the user while computer system 600 is authorized to access the account (e.g., computer system 600 is logged into, is authorized for use with, and/or is able to access the account). In some embodiments, the user of computer system 600 configures and/or authorizes computer system 600 to collect, track, receive, predict, estimate, and/or display information related to the menstrual cycle of the user.

In some embodiments, computer system 600 receives information (e.g., via one or more user inputs) indicating that the user of the account corresponding to computer system 600 is pregnant. As set forth below, computer system 600 is configured to display at least a portion of cycle tracking user interface 606 with different appearances based on a pregnancy designation of the account corresponding to computer system 600. For instance, at FIG. 8A, computer system 600 determines that the account does not include a pregnancy designation and/or determines that the account does not include information related to the user of the account currently experiencing pregnancy. As set forth below with reference to FIGS. 8E-8L, computer system 600 displays at least a portion of cycle tracking user interface 606 with a different appearance (e.g., a different appearance than cycle tracking user interface 606 shown at FIG. 8A) based on a determination that the account includes a pregnancy designation (e.g., the user of the account is currently pregnant).

At FIG. 8A, cycle tracking user interface 606 includes cycle tracking region 610, which includes cycle tracking indicators 610a-610i corresponding to respective days. Cycle tracking indicators 610a-610i provide visual indications of information about the menstrual cycle of the user of the account corresponding to computer system 600. For example, cycle tracking indicators 610a-610i visually indicate whether the user was and/or is currently experiencing a menstrual cycle on a respective day, whether the user experienced a menstrual cycle symptom on a respective day, whether the user experienced bleeding on a respective day, and/or fertility information for a respective day.

At FIG. 8A, cycle tracking indicator 610e is currently selected, as indicated by computer system 600 displaying cycle tracking indicator 610e at a larger size as compared to cycle tracking indicators 610a-610d and/or 610f-610i. At FIG. 8A, cycle tracking indicator 610e corresponds to the current day (e.g., the current day at which computer system 600 displays cycle tracking user interface 606). In some embodiments, computer system 600 is configured to add and/or associate information with a respective cycle tracking indicator 610a-610i in response to detecting selection of a respective cycle tracking indicator 610a-610i. For example, in some embodiments, in response to detecting selection of a respective cycle tracking indicator 610a-610i while the account corresponding to computer system 600 does not include a pregnancy designation (e.g., the user of the account has not provided information indicating that they are pregnant), computer system 600 displays the respective cycle tracking indicator 610a-610i with a menstrual cycle indicator. In some embodiments, computer system 600 determines, logs, records, tracks, and/or otherwise includes information that the user of the account experienced and/or is experiencing their menstrual cycle on the respective day corresponding to the selected cycle tracking indicator 610a-610i.

At FIG. 8A, cycle tracking user interface 606 includes period user interface object 606a, symptom user interface object 606b, spotting user interface object 606c, and factors user interface object 606d. In some embodiments, computer system 600 determines, logs, records, and/or otherwise includes information that the user experienced and/or is experiencing their menstrual cycle on the respective day corresponding to the selected cycle tracking indicator 610a-610i (e.g., cycle tracking indicator 610e at FIG. 8A) in response to detecting selection of period user interface object 606a. In some embodiments, computer system 600 displays a menstrual cycle user interface in response to detecting selection of period user interface object 606a. In some embodiments, computer system 600 displays a symptoms user interface (e.g., a user interface that includes selectable options corresponding to symptoms that a user experiences during a menstrual cycle) in response to detecting selection of symptom user interface object 606b and/or displays a symptom indicator on the selected cycle tracking indicator 610a-610i. In some embodiments, computer system 600 displays a spotting user interface (e.g., a user interface with selectable spotting/bleeding options that a user experiences during a menstrual cycle) in response to detecting selection of spotting user interface object 606c and/or displays a spotting indicator on the selected cycle tracking indicator 610a-610i. In some embodiments, in response to detecting selection of factors user interface object 606d, computer system 600 displays factors user interface 612.

At FIG. 8A, computer system 600 displays pregnancy designation user interface object 606e on cycle tracking user interface 606. In some embodiments, computer system 600 displays pregnancy designation user interface object 606e based on information received by computer system 600 (e.g., a positive pregnancy test result). In some embodiments, computer system 600 displays pregnancy designation user interface object 606e based on information related to the menstrual cycle of the user of the account (e.g., the user has indicated that they have not experienced their menstrual cycle for a threshold amount of time). In some embodiments, computer system 600 displays pregnancy designation user interface object 606e as a default user interface object of cycle tracking user interface 606 (e.g., computer system 600 displays pregnancy designation user interface object 606e unless computer system 600 receives user input requesting to dismiss, clear, and/or remove display of pregnancy designation user interface object 606e).

At FIG. 8A, computer system 600 detects user input 850a (e.g., a swipe gesture, a touch input, an air gesture, a mouse click, a gaze input, and/or a speech input) corresponding to cycle tracking region 610. In response to detecting user input 850a, computer system 600 translates, moves, and/or removes display of cycle tracking indicators 610a-610i and displays cycle tracking indicators 610j-610r, as shown at FIG. 8B.

At FIG. 8B, cycle tracking indicators 610j and 610k include an appearance (e.g., as indicated by hatching at FIG. 8B) indicating that the user of the account corresponding to computer system 600 experienced a fertility event on the respective days corresponding to cycle tracking indicators 610j and 610k. In some embodiments, computer system 600 estimates and/or predicts a range of time in which the user of the account is fertile and/or more likely to become pregnant. In some embodiments, computer system 600 estimates and/or predicts the fertility range of time based on information received and/or input via the user related to their menstrual cycle. For instance, in some embodiments, computer system 600 determines a pattern of a menstrual cycle of the user based on information received by computer system 600 (e.g., information received via one or more inputs detected by computer system 600). In some embodiments, computer system 600 determines the fertility range of time based on an estimated date and/or a logged date of a menstrual cycle of the user (e.g., computer system 600 estimates the fertility range of time to be within 6-10 days after a menstrual cycle ends). As such, the user of computer system 600 can view cycle tracking user interface 606 and determine dates and/or times at which the user would most likely become pregnant.

At FIG. 8B, cycle tracking indicators 610m and 610n include menstruation indicators 800 indicating that the user of the account corresponding to computer system 600 experienced and/or is likely to experience their menstrual cycle on the respective days corresponding to cycle tracking indicators 610m and 610n. In some embodiments, computer system 600 displays menstruation indicators 800 within and/or on cycle tracking indicators 610m and/or 610n based on one or more user inputs (e.g., one or more user inputs directed to cycle tracking indicators 610m and/or 610n). In some embodiments, computer system 600 estimates and/or predicts that the user is likely to experience their menstrual cycle on the respective days corresponding to cycle tracking indicators 610m and/or 610n based on past information corresponding to the menstrual cycle of the user.

In some embodiments, computer system 600 predicts and/or estimates the fertility range of time for the user and/or predicts and/or estimates when a user is likely to experience their menstrual cycle based on information received by computer system 600. For instance, in some embodiments, computer system 600 detects one or more user inputs corresponding to a request to add, log, and/or record menstrual cycle information. In some embodiments, based on past information received and/or detected by computer system 600, computer system 600 predicts and/or estimates future events related to the menstrual cycle of the user.

At FIG. 8B, computer system 600 detects user input 850b (e.g., a tap input, a touch input, an air gesture, a mouse click, a gaze input, and/or a speech input) corresponding to cycle tracking indicator 610l. In response to detecting user input 850b, computer system 600 selects cycle tracking indicator 610l. In some embodiments, computer system 600 displays cycle tracking indicator 610l within a center region of cycle tracking region 610 in response to detecting user input 850b. In some embodiments, computer system 600 enlarges a size of cycle tracking indicator 610l and reduces a size of cycle tracking indicator 610n in response to user input 850b to indicate that cycle tracking indicator 610l has been selected. In some embodiments, computer system 600 displays menstruation bleeding user interface 802 in response to detecting user input 850b. In some embodiments, after detecting user input 850b, computer system 600 detects user input 850c (e.g., a tap input, a touch input, an air gesture, a mouse click, a gaze input, and/or a speech input) corresponding to add period user interface object 606f of cycle tracking user interface 606. In some embodiments, in response to detecting user input 850c, computer system 600 displays menstruation bleeding user interface 802, as shown at FIG. 8C.

At FIG. 8C, menstruation bleeding user interface 802 includes selectable options that enable computer system 600 to record and/or log information related to a menstrual cycle of the user. For instance, at FIG. 8C, menstruation bleeding user interface 802 includes selectable period options 802a and 802b. Selectable period option 802a (e.g., “Had Flow”) corresponds to the user experiencing their menstrual period on the respective day corresponding to cycle tracking indicator 610l. Selectable period option 802b (e.g., “No Flow”) corresponds to the user not having experienced their menstrual period on the respective day corresponding to cycle tracking indicator 610l.

Menstruation bleeding user interface 802 also includes selectable bleeding options 802c-802e, which provide additional information about the menstrual cycle of the user for the respective day corresponding to cycle tracking indicator 610l. For instance, at FIG. 8C, selectable bleeding option 802c corresponds to a light flow of bleeding on the respective day, selectable bleeding option 802d corresponds to a medium flow of bleeding on the respective day, and selectable bleeding option 802e corresponds to a heavy flow of bleeding on the respective day. In some embodiments, computer system 600 displays selectable bleeding options 802c-802e with a first appearance (e.g., a grayed-out appearance and/or a different color from selectable period options 802a and 802b) when selectable period option 802a is not selected (and/or when selectable period option 802b is selected) to indicate that selectable bleeding options 802c-802e cannot be selected. In some embodiments, computer system 600 displays selectable bleeding options 802c-802e with a second appearance (e.g., a non-grayed-out appearance and/or the same color as selectable period options 802a and 802b) when selectable period option 802a is selected (and/or when selectable period option 802b is not selected) to indicate that selectable bleeding options 802c-802e can be selected.

At FIG. 8C, computer system 600 detects user input 850d (e.g., a tap input, a touch input, an air gesture, a mouse click, a gaze input, and/or a speech input) corresponding to selectable period option 802a. In some embodiments, in response to detecting user input 850d, computer system 600 displays a selection indicator corresponding to selectable period option 802a to confirm selection of selectable period option 802a. In some embodiments, computer system 600 displays selectable bleeding options 802c-802e with the second appearance in response to detecting user input 850d. In addition, at FIG. 8C, computer system 600 detects user input 850e (e.g., a tap input, a touch input, an air gesture, a mouse click, a gaze input, and/or a speech input) corresponding to selectable bleeding option 802d. In some embodiments, in response to detecting user input 850e, computer system displays a selection indicator corresponding to selectable bleeding option 802d to confirm selection of selectable bleeding option 802d. After detecting user inputs 850d and/or 850e, computer system 600 detects user input 850f (e.g., a tap input, a touch input, an air gesture, a mouse click, a gaze input, and/or a speech input) corresponding to done user interface object 802f of menstruation bleeding user interface 802. In response to detecting user input 850f, computer system 600 displays (e.g., re-displays) cycle tracking user interface 606, as shown at FIG. 8D.

At FIG. 8D, computer system 600 displays cycle tracking user interface 606 with cycle tracking indicator 610l currently selected in cycle tracking region 610, as indicated by the larger size of cycle tracking indicator 610l as compared to the remaining cycle tracking indicators of cycle tracking region 610. In addition, at FIG. 8D, computer system displays menstruation indicator 800 in cycle tracking indicator 610l based on user inputs 850d, 850e, and/or 850f. For instance, user inputs 850d, 850e, and/or 850f corresponded to information that the user of the account corresponding to computer system 600 experienced a medium flow of bleeding during their menstrual cycle on the respective day corresponding to cycle tracking indicator 610l. Based on the information corresponding to user inputs 850d, 850e, and/or 850f, computer system 600 updates display of cycle tracking indicator 610l to include menstruation indicator 800 to indicate that the user experienced their menstrual cycle on the respective day corresponding to cycle tracking indicator 610l.

As set forth above, computer system 600 displays one or more portions of cycle tracking user interface 606 with a different appearance based on whether computer system 600 determines that the account includes a pregnancy designation. For instance, at FIG. 8D, computer system 600 detects user input 850g (e.g., a tap input, a touch input, an air gesture, a mouse click, a gaze input, and/or a speech input) corresponding to pregnancy designation user interface object 606e. In response to detecting user input 850g, computer system 600 displays pregnancy user interface 614 and initiates a process for adding the pregnancy designation to the account corresponding to computer system 600, as described in detail above with reference to FIGS. 6G-6T. After adding the pregnancy designation to the account corresponding to computer system 600, computer system 600 displays cycle tracking user interface 606, as shown at FIG. 8E.

At FIG. 8E, computer system 600 has added the pregnancy designation to the account and displays cycle tracking user interface 606. At FIG. 8E, cycle tracking indicator 610e is currently selected, as indicated by computer system 600 displaying cycle tracking indicator 610e at a size that is larger than the size of cycle tracking indicators 610a-610d and 610f-610i. At FIG. 8E, cycle tracking indicator 610e corresponds to the current day (e.g., the current day at which computer system 600 is displaying cycle tracking user interface 606).

At FIG. 8E, cycle tracking user interface 606 includes cycle tracking indicators 610e-610i, which include a different appearance as compared to cycle tracking indicators 610e-610i shown at FIG. 8A. For instance, cycle tracking indicator 610e, which corresponds to the current day, includes outline 804a, indicating that the user of the account corresponding to computer system 600 is pregnant on the respective day (e.g., the current day) corresponding to cycle tracking indicator 610e. In addition, cycle tracking indicators 610f-610i include outline 804b indicating that the user of the account corresponding to computer system 600 is expected to be and/or will be pregnant on the respective days corresponding to cycle tracking indicators 610f-610i. As such, computer system 600 displays cycle tracking indicators 610e-610f with outlines 804a and/or 804b to provide a visual indication that the user is pregnant and/or is expected to be pregnant on the respective days corresponding to cycle tracking indicators 610e-610f. At FIG. 8E, computer system 600 displays cycle tracking indicator 610e with a different outline (e.g., outline 804a instead of 804b) to visually indicate which day is the current day during the pregnancy event.

At FIG. 8E, computer system 600 displays bleeding user interface object 810a, symptom user interface object 810b, and factors user interface object 810c. Bleeding user interface object 810a allows computer system 600 to receive (e.g., via one or more user inputs) information related to bleeding experienced by the user during the pregnancy event. As set forth below with reference to FIGS. 8H-8J, computer system 600 displays different options for bleeding during pregnancy as compared to the options (e.g., options 802a-802e shown at FIG. 8C) for bleeding during a menstrual cycle. Symptoms user interface object 810b enables computer system 600 to receive (e.g., via one or more inputs) information related to symptoms (e.g., symptoms other than bleeding) experienced by the user during the pregnancy event, as described below with reference to FIGS. 8G and 8H. Computer system 600 displays factors user interface object 810c with an indication (e.g., text) that the user of the account corresponding to computer system 600 is currently pregnant.

At FIG. 8E, computer system 600 determines that the pregnancy event of the user of the account started on the current day (e.g., July 22^{nd}). Accordingly, computer system 600 displays cycle tracking indicators 610a-610d with the same appearance as cycle tracking indicators 610a-610d shown at FIG. 8A. As such, cycle tracking indicators 610a-610d indicate that the user of the account corresponding to computer system 600 was not pregnant on the respective days corresponding to cycle tracking indicators 610a-610d. Thus, computer system 600 displays cycle tracking indicators 610a-610r with different appearances based on whether computer system 600 determines that the account includes the pregnancy designation and/or based on whether the pregnancy event occurred, is occurring, will occur, and/or is expected to occur on a respective day corresponding to cycle tracking indicators 610a-610r.

At FIG. 8E, computer system 600 displays pregnancy timeline 806 on cycle tracking user interface 606 based on the pregnancy designation of the account corresponding to computer system 600. Pregnancy timeline 806 includes estimated age indicator 806a, trimester indicator 806b, timeline 808, estimated start date indicator 806c, and estimated due date indicator 806d. As set forth above with reference to FIGS. 6G-61, computer system 600 estimates and/or predicts various information about a pregnancy event based on information received by computer system 600 (e.g., via one or more user inputs). Computer system 600 displays pregnancy timeline 806 with information that is based on the information received by computer system 600 and/or based on the estimated information determined and/or calculated by computer system 600.

As set forth above, computer system 600 determines that the pregnancy event began on the current day (e.g., July 22^{nd}). Accordingly, estimated age indicator 806a includes an indication (e.g., text) that the age of the pregnancy event is one day. In addition, computer system 600 displays trimester indicator 806b with an indication that the pregnancy event is estimated to be in the first trimester. Estimated start date indicator 806c includes an indication (e.g., text) that the estimated start date of the pregnancy event is the current date of July 22^{nd}. Further, estimated due date indicator 806d includes an indication (e.g., text) indicating that the estimated due date of the pregnancy event (e.g., when the user is predicted and/or expected to give birth) is April 14^{th}.

At FIG. 8E, timeline 808 includes current date indicator 808a and pregnancy duration indicator 808b. At FIG. 8E, current date indicator 808a includes a dot and/or a thumb indicating a current date relative to the entire duration of the pregnancy. The entire duration of the pregnancy is indicated by duration indicator 808b, which is displayed as a bar and/or line at FIG. 8E. Thus, computer system 600 provides a visual indication of how far along the pregnancy event has progressed compared to the estimated duration of the pregnancy event via timeline 808.

Computer system 600 is configured to update pregnancy timeline 806 over time and/or at the start of each day during the pregnancy event. For instance, at FIG. 8F, computer system 600 displays pregnancy timeline 806 at a time that is after the time shown at FIG. 8E (e.g., August 20^{th} instead of July 22^{nd}). At FIG. 8F, estimated age indicator 806a includes an indication (e.g., text) that the age of the pregnancy event is three weeks and five days. In addition, computer system 600 displays trimester indicator 806b with an indication that the pregnancy event is estimated to still be in the first trimester. Because computer system 600 does not receive any additional information related to the pregnancy event, computer system 600 maintains display of estimated start date indicator 806c with the indication that the estimated start date of the pregnancy event is July 22^{nd} and maintains display of estimated due date indicator 806d with an indication that the estimated due date of the pregnancy event is April 14^{th}.

At FIG. 8F, computer system updates timeline 808 so that current date indicator 808a has moved from the position shown at FIG. 8E. For instance, at FIG. 8F, current date indicator 808a is displayed at a position that is to the right of the position shown at FIG. 8E relative to duration indicator 808b. Therefore, the user of computer system 600 can quickly determine how far along the pregnancy has progressed by viewing pregnancy timeline 806.

At FIG. 8F, computer system 600 displays cycle tracking indicators 610s-610aa within cycle tracking region 610 of cycle tracking user interface 606. At FIG. 8F, cycle tracking indicator 610w includes outline 804a indicating that cycle tracking indicator 610w corresponds to the current date (e.g., the current date at which computer system 600 is displaying cycle tracking user interface 606). Computer system 600 displays cycle tracking indicators 610s-610v and 610x-610aa with outline 804b indicating that the user of the account corresponding to computer system 600 was and/or is expected to be pregnant on the respective days corresponding to cycle tracking indicators 610s-610v and 610x-610aa.

As set forth above, cycle tracking user interface 606 includes symptom user interface object 810b, which allows computer system 600 to receive information related to symptoms experienced by the user during the pregnancy event. For instance, at FIG. 8F, computer system 600 detects user input 850h (e.g., a tap input, a touch input, an air gesture, a mouse click, a gaze input, and/or a speech input) corresponding to symptom user interface object 810b. In response to detecting user input 850h, computer system 600 displays symptoms user interface 812, as shown at FIG. 8G.

At FIG. 8G, computer system 600 displays symptoms user interface 812, which includes selectable symptom options 812a-812g. Selectable symptom options 812a-812g correspond to common symptoms experienced by people during pregnancy. In some embodiments, computer system 600 displays additional selectable symptom options in response to detecting user input (e.g., a swipe gesture) corresponding to symptoms user interface 812. At FIG. 8G, computer system 600 detects user input 850i (e.g., a tap input, a touch input, an air gesture, a mouse click, a gaze input, and/or a speech input) corresponding to selectable symptom option 812c. In some embodiments, in response to detecting user input 850i, computer system 600 displays a selection indicator corresponding to selectable symptom option 812c. After detecting user input 850i, computer system 600 detects user input 850j (e.g., a tap input, a touch input, an air gesture, a mouse click, a gaze input, and/or a speech input) corresponding to done user interface object 812h of symptoms user interface 812. In response to detecting user input 850j, computer system 600 displays cycle tracking user interface 606 and updates cycle tracking indicator 610w to include symptom indicator 814, as shown at FIG. 8H.

At FIG. 8H, computer system 600 displays cycle tracking user interface 606 and updates cycle tracking indicator 610w to include symptom indicator 814 based on inputs 850i and/or 850j. Accordingly, computer system 600 provides a visual indication that computer system 600 received information indicating that user experienced a symptom (e.g., appetite changes) on the respective day corresponding to cycle tracking indicator 610w. In addition, computer system 600 displays symptom user interface object 810b with symptom description 817. In some embodiments, computer system 600 displays symptom user interface object 810b with symptom description 817 while cycle tracking indicator 610w is selected. In some embodiments, when a cycle tracking indicator that does not include symptom indicator 814 is selected, computer system 600 does not display symptom description 817 with symptom user interface object 810b.

As set forth above, bleeding user interface object 810a allows computer system 600 to receive information related to bleeding of the user of the account during the pregnancy event. At FIG. 8H, computer system 600 detects user input 850k (e.g., a tap input, a touch input, an air gesture, a mouse click, a gaze input, and/or a speech input) corresponding to bleeding user interface object 810a. In response to detecting user input 850k, computer system 600 displays pregnancy bleeding user interface 816, as shown at FIG. 8I.

At FIG. 8I, pregnancy bleeding user interface 816 includes selectable options that enable computer system 600 to receive information and/or log information related to bleeding experienced by the user of the account during the pregnancy event. At FIG. 8I, pregnancy bleeding user interface 816 is different from menstruation bleeding user interface 802, as described above with reference to FIG. 8C. For instance, pregnancy bleeding user interface 816 includes pregnancy bleeding options 816a and 816b. Pregnancy bleeding option 816a (e.g., “Had Bleeding”) corresponds to the user experiencing bleeding during the pregnancy event on the respective day corresponding to cycle tracking indicator 610w. Pregnancy bleeding option 816b (e.g., “No Bleeding”) corresponds to the user not having experienced bleeding during the pregnancy event on the respective day corresponding to cycle tracking indicator 610w.

Pregnancy bleeding user interface 816 also includes selectable bleeding options 816c-816e, which provide additional information about the bleeding experienced by the user during the pregnancy event for the respective day corresponding to cycle tracking indicator 610w. For instance, at FIG. 8I, selectable bleeding option 816c corresponds to a light amount of bleeding on the respective day, selectable bleeding option 816d corresponds to a medium amount of bleeding on the respective day, and selectable bleeding option 816e corresponds to a heavy amount of bleeding on the respective day. In some embodiments, computer system 600 displays selectable bleeding options 816c-816e with a first appearance (e.g., a grayed-out appearance and/or a different color from pregnancy bleeding options 816a and 816b) when pregnancy bleeding option 816a is not selected (and/or when pregnancy bleeding option 816b is selected) to indicate that selectable bleeding options 816c-816e cannot be selected. In some embodiments, computer system 600 displays selectable bleeding options 816c-816e with a second appearance (e.g., a non-grayed-out appearance and/or the same color as pregnancy bleeding options 816a and 816b) when pregnancy bleeding option 816a is selected (and/or when pregnancy bleeding option 816b is not selected) to indicate that selectable bleeding options 816c-816e can be selected.

At FIG. 8I, computer system 600 detects user input 850l (e.g., a tap input, a touch input, an air gesture, a mouse click, a gaze input, and/or a speech input) corresponding to pregnancy bleeding option 816a. In some embodiments, in response to detecting user input 850l, computer system 600 displays a selection indicator corresponding to pregnancy bleeding option 816a to confirm selection of pregnancy bleeding option 816a. In some embodiments, computer system 600 displays selectable bleeding options 816c-816e with the second appearance in response to detecting user input 850l. In addition, at FIG. 8I, computer system 600 detects user input 850m (e.g., a tap input, a touch input, an air gesture, a mouse click, a gaze input, and/or a speech input) corresponding to selectable bleeding option 816d. In some embodiments, in response to detecting user input 850m, computer system 600 displays a selection indicator corresponding to selectable bleeding option 816d to confirm selection of selectable bleeding option 816d. After detecting user inputs 850l and/or 850m, computer system 600 detects user input 850n (e.g., a tap input, a touch input, an air gesture, a mouse click, a gaze input, and/or a speech input) corresponding to done user interface object 816f of pregnancy bleeding user interface 816. In response to detecting user input 850n, computer system 600 displays bleeding information user interface 818, as shown at FIG. 8J.

At FIG. 8J, bleeding information user interface 818 includes information 818a indicating that bleeding experienced during pregnancy can be a sign of complications and/or suggesting that the user discuss the bleeding with their doctor. Computer system 600 displays bleeding information user interface 818 to notify the user of computer system 600 that the bleeding information received via user inputs 850l, 850m, and/or 850n is abnormal during the pregnancy event. As such, the user of computer system 600 is able to review information 818a and determine whether additional actions, such as calling their doctor, are necessary.

At FIG. 8J, computer system 600 detects user input 8500 (e.g., a tap input, a touch input, an air gesture, a mouse click, a gaze input, and/or a speech input) corresponding to done user interface object 818b of bleeding information user interface 818. In response to detecting user input 8500, computer system 600 displays (e.g., re-displays) cycle tracking user interface 606, as shown at FIG. 8K.

At FIG. 8K, computer system 600 displays cycle tracking user interface 606 with cycle tracking indicator 610w including bleeding indicator 820 based on computer system 600 receiving and/or detecting user inputs 850l, 850m, and/or 850n. Thus, computer system 600 provides a visual indication of whether a user experienced bleeding during the pregnancy event on a respective day by displaying bleeding indicator 820 on a respective cycle tracking indicator.

At FIG. 8L, computer system 600 displays cycle tracking user interface 606 at a time that is after the time shown at FIGS. 8F-8J. For instance, at FIG. 8L, computer system 600 displays cycle tracking user interface 606 with cycle tracking indicator 610af selected, which corresponds to the current date (e.g., the current date at which computer system 600 is displaying cycle tracking user interface 606).

At FIG. 8L, estimated age indicator 806a includes an indication (e.g., text) that the age of the pregnancy event is 39 weeks and four days. In addition, computer system 600 displays trimester indicator 806b with an indication that the pregnancy event is estimated to be in the third trimester. Because computer system 600 does not receive any additional information related to the pregnancy event (e.g., between the time computer system 600 displays cycle tracking user interface 606 at FIG. 8J and the time at which computer system 600 displays cycle tracking user interface 606 at FIG. 8L), computer system 600 maintains display of estimated start date indicator 806c with the indication that the estimated start date of the pregnancy event is July 22^{nd} and maintains display of estimated due date indicator 806d with an indication that the estimated due date of the pregnancy event is April 14^{th}.

At FIG. 8L, computer system updates timeline 808 so that current date indicator 808a has moved from the position shown at FIGS. 8F, 8H, and 8K. For instance, at FIG. 8L, current date indicator 808a is displayed at a position that is to the right of the position shown at FIGS. 8F, 8H, and 8K relative to duration indicator 808b. Therefore, the user of computer system 600 can quickly determine how far along the pregnancy has progressed by viewing pregnancy timeline 806.

At FIG. 8L, computer system 600 displays cycle tracking indicators 610ab-610aj within cycle tracking region 610 of cycle tracking user interface 606. At FIG. 8L, cycle tracking indicator 610af includes outline 804a indicating that cycle tracking indicator 610w corresponds to the current date (e.g., the current date at which computer system 600 is displaying cycle tracking user interface 606). Computer system 600 displays cycle tracking indicators 610ab-610ae and 610ag-610ai with outline 804b indicating that the user of the account corresponding to computer system 600 was and/or is expected to be pregnant on the respective days corresponding to cycle tracking indicators 610ab-610ae and 610ag-610ai. Further, computer system 600 displays cycle tracking indicator 610ai with an appearance that is different from cycle tracking indicators 610ab-610ah. For instance, at FIG. 6L, cycle tracking indicator 610ai includes fill 822 in addition to outline 804b. Cycle tracking indicator 610ai corresponds to the estimated due date of the pregnancy event, and thus, computer system 600 displays cycle tracking indicator 610ai with a different appearance when compared to the other cycle tracking indicators. Accordingly, the user of computer system 600 can quickly determine when the estimated due date of the pregnancy event is estimated to occur based on the appearance of cycle tracking indicators. Further still, computer system 600 displays cycle tracking indicator 610aj with a different appearance from cycle tracking indicators 610ab-610ai. For instance, at FIG. 8L, cycle tracking indicator 610aj does not include fill 822 and does not include outline 804a and/or outline 804b. Cycle tracking indicator 610aj corresponds to a date that is after the estimated due date and/or a date at which computer system 600 determines that the user of the account is not expected to be pregnant. As such, computer system 600 displays cycle tracking indicator 610aj with the appearance shown at FIG. 8L, which is the same as the appearance of cycle tracking indicators 610a-610d, shown at FIG. 8E.

FIG. 9 is a flow diagram illustrating a method for tracking a pregnancy event using a computer system in accordance with some embodiments. Method 900 is performed at a computer system (e.g., 100, 300, 500, and/or 600) (e.g., a smart phone, a smart watch, a tablet computer, a laptop computer, a desktop computer, a wearable device, and/or a head-mounted device) that is in communication with one or more display generation components (e.g., 602) (e.g., one or more displays, touch-screen displays, monitors, holographic display systems, and/or head-mounted display systems) and one or more input devices (e.g., 602) (e.g., a touch-sensitive surface (e.g., a touch-sensitive display); a mouse; a keyboard; a remote control; a visual input device (e.g., one or more cameras such as, e.g., an infrared camera, a depth camera, a visible light camera, and/or a gaze tracking camera); an audio input device (e.g., a microphone); a biometric sensor (e.g., a fingerprint sensor, a face identification sensor, a gaze tracking sensor, and/or an iris identification sensor); and/or one or more mechanical input devices (e.g., a depressible input mechanism; a button; a rotatable input mechanism; a crown; and/or a dial)). Some operations in method 900 are, optionally, combined, the orders of some operations are, optionally, changed, and some operations are, optionally, omitted.

As described below, method 900 provides an intuitive way for tracking a pregnancy event. The method reduces the cognitive burden on a user for tracking a pregnancy event, thereby creating a more efficient human-machine interface. For battery-operated computing devices, enabling a user to track a pregnancy event faster and more efficiently conserves power and increases the time between battery charges.

The computer system (e.g., 100, 300, 500, and/or 600) detects (902), via the one or more input devices, user input (e.g., 650a) (e.g., a tap gestures, a swipe gesture, a touch gesture, an air gesture, a button press, and/or a voice command) corresponding to a request to display a cycle tracking user interface (e.g., 606) (e.g., a user interface that includes information related to a menstrual period of a user of the computer system, information related to fertility of a user of the computer system, information related to a pregnancy of a user of the computer system, and/or information related to lactation of a user of the computer system).

In response to detecting the user input (e.g., 650a) corresponding to the request to display the cycle tracking user interface (e.g., 606), the computer system (e.g., 100, 300, 500, and/or 600) displays (904), via the one or more display generation components (e.g., 602), the cycle tracking user interface (e.g., 606). The cycle tracking user interface (e.g., 606) includes a first cycle tracking indicator (e.g., 610a-610aj) (e.g., text, an image, an icon, and/or a graphical user interface object that corresponds to a respective duration, such as a day, that includes information about and/or an appearance indicative of characteristics of one or more menstrual periods, fertility, pregnancy, and/or lactation of a user of the computer system) corresponding to a first day (e.g., the first cycle tracking indicator does not correspond to a second day); and a second cycle tracking indicator (e.g., 610a-610aj) (e.g., text, an image, an icon, and/or a graphical user interface object that corresponds to a respective duration, such as a day, that includes information about and/or an appearance indicative of characteristics of one or more menstrual periods, fertility, pregnancy, and/or lactation of a user of the computer system) corresponding to a second day that is different from the first day (e.g., the second cycle tracking indicator does not correspond to the first day).

Displaying the cycle tracking user interface (e.g., 606) includes: in accordance with a determination that an account (e.g., an account and/or user profile of a user of the computer system that is linked to, synced to, and/or otherwise accessible by the computer system so that the computer system can display information related to the account on the cycle tracking user interface) corresponding to the cycle tracking user interface (e.g., 606) (e.g., the account is accessible by the computer system so that the computer system can display information related to the account on the cycle tracking user interface) includes an indication that a user of the account is pregnant (e.g., the account includes a designation, such as a user-defined designation received via one or more user inputs, indicating that the user of the account is currently pregnant and/or was pregnant on the first day) on the first day, the first cycle tracking indicator (e.g., 610a-610aj) has a first appearance (906) (e.g., 804a, 804b, and/or 822) (e.g., a first color, a first shading, a first outline, a first size, and/or a first shape). In some embodiments, the first appearance includes an appearance that indicates and/or provides visual feedback to a user of the computer system (e.g., 100, 300, 500, and/or 600) that the user is pregnant and/or was pregnant at a particular time.

Displaying the cycle tracking user interface (e.g., 606) includes: in accordance with a determination that the account corresponding to the cycle tracking user interface (e.g., 606) does not include an indication that the user of the account is pregnant (e.g., the account does not include and/or the computer system has not received a designation that the user of the computer system is pregnant on the first day) on the first day, the first cycle tracking indicator (e.g., 610a-610aj) has a second appearance (908) (e.g., no outline 804a, no outline 804b, and/or no fill 822) (e.g., a second color, a second shading, a second outline, a second size, and/or a second shape) that is different from the first appearance (e.g., 804a, 804b, and/or 822).

Displaying the cycle tracking user interface (e.g., 606) includes: in accordance with a determination that the account corresponding to the cycle tracking user interface (e.g., 606) includes an indication that the user of the account is pregnant on the second day (e.g., the account includes a designation, such as a user-defined designation received via one or more user inputs, indicating that the user of the account is currently pregnant and/or was pregnant on the second day), the second cycle tracking indicator (e.g., 610a-610aj) has the first appearance (910) (e.g., 804a, 804b, and/or 822).

Displaying the cycle tracking user interface (e.g., 606) includes: in accordance with a determination that the account corresponding to the cycle tracking user interface (e.g., 606) does not include an indication that the user of the account is pregnant on the second day (e.g., the account does not include and/or the computer system has not received a designation that the user of the computer system is pregnant on the second day), the second cycle tracking indicator (e.g., 610a-610aj) has the second appearance (912) (e.g., no outline 804a, no outline 804b, and/or no fill 822) that is different from the first appearance (e.g., 804a, 804b, and/or 822). In some embodiments, the first appearance (e.g., 804a, 804b, and/or 822) includes a first color and/or first shading and the second appearance (e.g., no outline 804a, no outline 804b, and/or no fill 822) includes a second color and/or a second shading that are different from the first color and/or the first shading, respectively. In some embodiments, the second appearance (e.g., no outline 804a, no outline 804b, and/or no fill 822) includes an appearance that indicates and/or provides visual feedback to a user of the computer system (e.g., 100, 300, 500, and/or 600) that the user is not pregnant and/or that the user was not pregnant at a particular time.

Displaying the cycle tracking user interface with respective cycle tracking indicators corresponding to respective days including different appearances based on whether or not an account corresponding to the cycle tracking user interface includes an indication that the user of the account is pregnant on a respective day allows a user to quickly determine on which days the user is pregnant, thereby providing improved visual feedback to the user.

In some embodiments, in accordance with a determination that the account corresponding to the cycle tracking user interface (e.g., 606) includes the indication that the user of the account is pregnant on the first day and in accordance with a determination that the first day is within a threshold duration (e.g., within one day, within one week, within two weeks, or within one month) from an estimated due date (e.g., 614b) of a pregnancy of the user of the account (e.g., an approximate, predicted, and/or projected date at which the user corresponding to the account of the computer system will and/or is likely to give birth), the first appearance (e.g., 804a,804b, and/or 822) of the first cycle tracking indicator (e.g., 610a-610aj) includes a first set of visual characteristics (e.g., 822) (e.g., a first color, a first shading, a first outline, a first fill, a first brightness, a first shape, and/or a first size). In accordance with a determination that the account corresponding to the cycle tracking user interface (e.g., 606) includes the indication that the user of the account is pregnant on the first day and in accordance with a determination that the first day is not within the threshold duration (e.g., the first day is further than one day, one week, two weeks, or one month from) from the estimated due date (e.g., 614b) of the pregnancy of the user of the account, the first appearance (e.g., 804a, 804b, and/or 822) of the first cycle tracking indicator (e.g., 610a-610aj) includes a second set of visual characteristics (e.g., 804a and/or 804b) (e.g., a second color, a second shading, a second outline, a second fill, a second brightness, a second shape, and/or a second size) that is different from the first set of visual characteristics (e.g., 822). Displaying the first cycle tracking indicator with different visual characteristics based on whether the first day is within a threshold duration from an estimated due date of a pregnancy of a user of the account or not allows a user to quickly determine how close the first day is to the estimated due date, thereby providing improved visual feedback to the user.

In some embodiments, in accordance with a determination that the first day is a current day (e.g., the current day at which the computer system displays the first cycle tracking indicator), the first appearance (e.g., 804a, 804b, and/or 822) of the first cycle tracking indicator (e.g., 610a-610aj) includes a solid outline (e.g., 804a) (e.g., a solid line around a perimeter of the first cycle tracking indicator). The first cycle tracking indicator including a solid outline when the first day is a current day allows a user to quickly determine that information corresponding to the first cycle tracking indicator relates to the current day, thereby providing improved visual feedback to the user.

In some embodiments, in accordance with a determination that the first day is a future date (e.g., a date that is in the future relative to a current date and/or a date that is after a current date at which the computer system displays the first cycle tracking indicator), the first appearance (e.g., 804a, 804b, and/or 822) of the first cycle tracking indicator (e.g., 610a-610aj) includes a dotted line outline (e.g., 804b) (e.g., a line that includes breaks to form dots around the perimeter of the first cycle tracking indicator). The first cycle tracking indicator including a dotted line outline when the first day is a future date allows a user to quickly determine that information corresponding to the first cycle tracking indicator relates to a time in the future, thereby providing improved visual feedback to the user.

In some embodiments, in accordance with a determination that the first day is the estimated due date (e.g., 614b) of the pregnancy of the user of the account, the first appearance (e.g., 804a, 804b, and/or 822) of the first cycle tracking indicator (e.g., 610a-610aj) includes a dotted line outline (e.g., 804b) (e.g., a line that includes breaks to form dots around the perimeter of the first cycle tracking indicator) and a solid fill (e.g., 822) (e.g., an interior region of the first cycle tracking indicator includes a color and/or shading that is solid within the interior region of the first cycle tracking indicator). The first cycle tracking indicator including a dotted line outline when the first day is the estimated due date of the pregnancy of the user of the account allows a user to quickly determine that information corresponding to the first cycle tracking indicator relates to the estimated due date, thereby providing improved visual feedback to the user.

In some embodiments, in response to detecting, via the one or more input devices (e.g., 602), one or more user inputs (e.g., 850h, 850i, and/or 850j) corresponding to a request to (e.g., one or more tap gestures, one or more swipe gestures, one or more touch gestures, one or more air gestures, one or more button presses, and/or one or more voice commands) associate a symptom (e.g., abdominal cramps, acne, appetite changes, bladder incontinence, bloating, breast pain, chills, constipation, diarrhea, dry skin, fatigue, hair loss, headache, hot flashes, lower back pain, memory lapse, mood changes, nausea, night sweats, pelvic pain, sleep changes, vaginal dryness, and/or another symptom associated with pregnancy) with a respective cycle tracking indicator (e.g., 610a-610aj) (e.g., the first cycle tracking indicator, the second cycle tracking indicator, or a third cycle tracking indicator that is different from the first cycle tracking indicator and the second cycle tracking indicator), the computer system (e.g., 100, 300, 500, and/or 600) displays, via the one or more display generation components (e.g., 602), the respective cycle tracking indicator (e.g., 610a-610aj) with a symptom indicator (e.g., 814) (e.g., the respective cycle tracking indicator includes a user interface object, such as an icon, a shape, an image, an object, and/or text, within an area of and/or proximate to the respective cycle tracking indicator that indicates that the user corresponding to the account of the computer system experienced a symptom on the respective day corresponding to the respective cycle tracking indicator). Displaying the respective cycle tracking indicator with a symptom indicator in response to detecting a request to associate a symptom with the respective cycle tracking indicator allows a user to quickly determine which days the user experienced a symptom, thereby providing improved visual feedback to the user.

In some embodiments, in response to detecting, via the one or more input devices (e.g., 602), one or more user inputs (e.g., 850c, 850d, 850e, 850f, 850k, 850l, 850m, and/or 850n) corresponding to a request to (e.g., one or more tap gestures, one or more swipe gestures, one or more touch gestures, one or more air gestures, one or more button presses, and/or one or more voice commands) associate bleeding (e.g. having or not having bleeding during a menstrual cycle, having or not having bleeding during pregnancy, and/or having or not having vaginal bleeding, such as low bleeding, medium bleeding, or heavy bleeding) with a respective cycle tracking indicator (e.g., 610a-610aj) (e.g., the first cycle tracking indicator, the second cycle tracking indicator, or a third cycle tracking indicator that is different from the first cycle tracking indicator and the second cycle tracking indicator), the computer system (e.g., 100, 300, 500, and/or 600) displays, via the one or more display generation components (e.g., 602), the respective cycle tracking indicator (e.g., 610a-610aj) with a bleeding indicator (e.g., 800 and/or 820) (e.g., the respective cycle tracking indicator includes a user interface object, such as an icon, a shape, an image, an object, and/or text, within an area of and/or proximate to the respective cycle tracking indicator that indicates that the user corresponding to the account of the computer system experienced bleeding or no bleeding on the respective day corresponding to the respective cycle tracking indicator). Displaying the respective cycle tracking indicator with a bleeding indicator in response to detecting a request to associate bleeding with the respective cycle tracking indicator allows a user to quickly determine which days the user experienced bleeding, thereby providing improved visual feedback to the user.

In some embodiments, in response to detecting the one or more user inputs (e.g., 850c, 850d, 850e, 850f, 850k, 850l, 850m, and/or 850n) corresponding to the request to associate bleeding with the respective cycle tracking indictor (e.g., 610a-610aj) and in accordance with a determination that the account corresponding to the cycle tracking user interface (e.g. 606) includes the indication that the user of the account is pregnant on a respective day that corresponds to the respective cycle tracking indicator (e.g., 610a-610aj) (e.g., the account includes a designation, such as a user-defined designation received via one or more user inputs, indicating that the user of the account is currently pregnant and/or was pregnant on the respective day that corresponds to the respective cycle tracking indicator), the computer system (e.g., 100, 300, 500, and/or 600) displays, via the one or more display generation components (e.g., 602), one or more first bleeding options (e.g., 816a-816e) (e.g., had bleeding, did not have bleeding, light bleeding, medium bleeding, or heavy bleeding). In response to detecting the one or more user inputs (e.g., 850c, 850d, 850e, 850f, 850k, 850l, 850m, and/or 850n) corresponding to the request to associate bleeding with the respective cycle tracking indictor (e.g., 610a-610aj) and in accordance with a determination that the account corresponding to the cycle tracking user interface (e.g., 606) does not include the indication that the user of the account is pregnant on the respective day that corresponds to the respective cycle tracking indicator (e.g., 610a-610aj) (e.g., the account does not include a designation, such as a user-defined designation received via one or more user inputs, indicating that the user of the account is currently pregnant and/or was pregnant on the respective day that corresponds to the respective cycle tracking indicator), the computer system (e.g., 100, 300, 500, and/or 600) displays, via the one or more display generation components (e.g., 602), one or more second bleeding options (e.g., 802a-802e) (e.g., had flow, did not have flow, light flow, medium flow, and/or heavy flow) that are different from the one or more first bleeding options (e.g., 816a-816e) (e.g., the one or more second bleeding options correspond to bleeding options of a menstrual cycle and the one or more first bleeding options correspond to bleeding options of a pregnancy). Displaying the one or more first bleeding options when the account includes the indication that the user is pregnant on a respective day that corresponds to a respective cycle tracking indicator and displaying the one or more second bleeding options when the account does not include the indication that the user is pregnant on the respective day that corresponds to the respective cycle tracking indicator allows the computer system to provide options to the user that are relevant to the current condition of the user, thereby performing an operation when a set of conditions has been met without requiring further user input.

In some embodiments, the one or more first bleeding options (e.g., 816a-816e) include: a first option (e.g., 816a) (e.g., a first selectable user interface object) corresponding to the user of the account having bleeding (e.g., the user experienced vaginal bleeding while pregnant on the respective day) on the respective day that corresponds to the respective cycle tracking indicator (e.g., 610-610aj), and a second option (e.g., 816b) (e.g., a second selectable user interface object) corresponding to the user of the account not having bleeding (e.g., the user did not experience vaginal bleeding while pregnant on the respective day) on the respective day that corresponds to the respective cycle tracking indicator (e.g., 610a-610aj). In some embodiments, the first option (e.g., 816a) includes one or more sub-options (e.g., 816c-816d), such as a first sub-option (e.g., 816c) corresponding to light bleeding, a second sub-option (e.g., 816d) corresponding to medium bleeding, and/or a third sub-option (e.g., 816e) corresponding to heavy bleeding. Displaying the first option corresponding to the user having bleeding on the respective day corresponding to the respective cycle tracking indicator and displaying the second option corresponding to the user not having bleeding on the respective day that corresponds to the respective cycle tracking indicator provides the user with options that are relevant to the current condition of the user, thereby performing an operation when a set of conditions has been met without requiring further user input.

In some embodiments, while displaying a respective cycle tracking indicator (e.g., 610a-610aj) (e.g., the first cycle tracking indicator, the second cycle tracking indicator, and/or a third cycle tracking indicator that is different from the first cycle tracking indicator and the second cycle tracking indicator) with the first appearance (e.g., 804a, 804b, and/or 822) (e.g., the appearance indicating that the user corresponding to the account of the computer system is pregnant on the respective day corresponding to the respective cycle tracking indicator), the computer system (e.g., 100, 300, 500, and/or 600) displays, via the one or more display generation components (e.g., 602), a timeline (e.g., 806 and/or 808) (e.g., a graphical user interface object that visually indicates a start date of the pregnancy, a completion date and/or estimated due date of the pregnancy, and/or a current date, such a as a progress bar and/or line) corresponding to a pregnancy of the user of the account. Displaying the timeline corresponding to the pregnancy of the user of the account provides the user with a visual indication of when the pregnancy started and/or when the pregnancy is expected and/or estimated to be completed relative to a current date, thereby providing improved visual feedback to the user.

In some embodiments, the first appearance (e.g., 804a, 804b, and/or 822) includes shading (e.g., 822) (e.g., hatching, a pattern, and/or a color fill within an area of the respective cycle tracking indicator that includes the first appearance), and the second appearance (e.g., no outline 804a, no outline 804b, and/or no fill 822) does not include shading (e.g., no fill 822) (e.g., the respective cycle tracking indicator having the second appearance does not include hatching, a pattern, and/or a color fill and/or the respective cycle tracking indicator having the second appearance includes a fill that is based on a background of the cycle tracking user interface and/or a white color fill). Displaying respective cycle tracking indicators with the first appearance, which includes shading, and displaying respective cycle tracking indicators with the second appearance, which does not include shading, allows a user to quickly determine on which days the user is pregnant, thereby providing improved visual feedback to the user.

In some embodiments, the first appearance (e.g., 804a, 804b, and/or 822) includes an outline (e.g., 804a and/or 804b) (e.g., a line, such as a solid line, a dotted line, or a dashed line, that includes a color and/or an emphasis, such as bolding, around a perimeter of the respective cycle tracking indicator that includes the first appearance), and the second appearance (e.g., no outline 804a, no outline 804b, and/or no fill 822) does not include the outline (e.g., no outline 804a and/or no outline 804b) (e.g., the respective cycle tracking indicator having the second appearance does not include a line having a color and/or an emphasis around the perimeter of the respective cycle tracking indicator). Displaying respective cycle tracking indicators with the first appearance, which includes an outline, and displaying respective cycle tracking indicators with the second appearance, which does not include the outline, allows a user to quickly determine on which days the user is pregnant, thereby providing improved visual feedback to the user.

Note that details of the processes described above with respect to method 900 (e.g., FIG. 9) are also applicable in an analogous manner to the methods described below/above. For example, methods 700 and 1100 optionally include one or more of the characteristics of the various methods described above with reference to method 900. For example, the computer system of method 700 can be used to add a pregnancy designation to an account of the computer systems of methods 900 and 1100. For brevity, these details are not repeated below.

FIGS. 10A-10P illustrate exemplary user interfaces for tracking health information during a pregnancy event, in accordance with some embodiments. The user interfaces in these figures are used to illustrate the processes described below, including the processes in FIG. 11.

FIG. 10A illustrates computer system 600 displaying, via display 602, health summary user interface 1000. At FIG. 10A, computer system 600 displays health summary user interface 1000 when computer system 600 determines that the account corresponding to computer system 600 does not include a pregnancy designation. Thus, computer system 600 displays health summary user interface 1000 at FIG. 10A while computer system 600 does not include information related to the user currently experiencing a pregnancy event and/or while the user of computer system 600 is not experiencing a pregnancy event.

At FIG. 10A, health summary user interface 1000 corresponds to the health application of computer system 600 (e.g., the same health application corresponding to health user interface 604). Health summary user interface 1000 includes favorites region 1002 and trends region 1004. Favorites region 1002 includes health information related to the user of the account corresponding to computer system 600. In some embodiments, favorites region 1002 includes health information that is user-defined and/or designated by the user of computer system 600. For example, in some embodiments, computer system 600 detects one or more user inputs requesting to include respective health information in favorites region 1002 and, in response to detecting the one or more user inputs, computer system 600 displays the respective health information in favorites region 1002. In some embodiments, favorites region 1002 includes health information that is system-defined, such as default health information that is included in favorites region 1002 by computer system 600. In some embodiments, favorites region 1002 includes a combination of user-defined and system-defined health information.

At FIG. 10A, favorites region 1002 includes weight platter 1002a, resting heart rate platter 1002b, and blood pressure platter 1002c. Weight platter 1002a includes information related to a weight of the user of the account corresponding to computer system 600, such as weight information received by computer system 600 via one or more user inputs and/or weight information received by computer system 600 via an external device (e.g., a scale that is in communication with computer system 600). Resting heart rate platter 1002b includes heart rate information of the user of the account corresponding to computer system 600 while the user is not active. In some embodiments, the resting heart rate information includes one or more heart rate measurements of the user and/or an average heart rate of a user while the user is inactive and/or relaxed for a threshold duration (e.g., one minute, two minutes, five minutes, or ten minutes). In some embodiments, computer system 600 receives resting heart rate information via an external device that is in communication with computer system 600, such as a smart watch. Blood pressure platter 1002c includes blood pressure information of the user corresponding to computer system 600. In some embodiments, computer system 600 receives the blood pressure information via user input and/or via an external device in communication with computer system 600 (e.g., a blood pressure sensor that is in communication with computer system 600).

At FIG. 10A, trends region 1004 includes patterns and/or relationships about health information over one or more periods of time. For instance, in some embodiments, computer system 600 is configured to receive health information over time either via user input and/or via an external device that is in communication with computer system 600. In some embodiments, the health information received by computer system 600 includes date and/or time information associated with a value of a respective health characteristic. In some embodiments, computer system 600 determines patterns and/or relationships about the health information over a respective duration of time (e.g., one day, one week, two weeks, one month, six months, or one year) to provide the user with an indication of how their health has and/or is changing. At FIG. 10A, trends region 1004 includes blood pressure trend platter 1004a corresponding to a pattern of blood pressure information over a respective duration of time (e.g., over 24 weeks). Blood pressure trend platter 1004a includes information indicating that the blood pressure information received by computer system 600 increased over the respective duration of time (e.g., over 24 weeks). As set forth above, computer system 600 displays blood pressure trend platter 1004a when computer system 600 determines that the account does not include a pregnancy designation for the user of the account. As set forth below, in some embodiments, computer system 600 includes further information related to trends about the health information when computer system 600 determines that the account includes a pregnancy designation.

At FIG. 10A, computer system 600 detects user input 1050a (e.g., a tap input, a touch input, an air gesture, a mouse click, a gaze input, and/or a speech input) corresponding to weight platter 1002a. In response to detecting user input 1050a, computer system 600 displays weight user interface 1006, as shown at FIG. 10B.

Alternatively, at FIG. 10A, computer system 600 detects user input 1050b (e.g., a tap input, a touch input, an air gesture, a mouse click, a gaze input, and/or a speech input) corresponding to resting heart rate platter 1002b. In response to detecting user input 1050b, computer system 600 displays resting heart rate user interface 1008, as shown at FIG. 10C. Alternatively, at FIG. 10A, computer system 600 detects user input 1050c (e.g., a tap input, a touch input, an air gesture, a mouse click, a gaze input, and/or a speech input) corresponding to blood pressure platter 1002c. In response to detecting user input 1050c, computer system 600 displays blood pressure user interface 1010, as shown at FIG. 10D. Alternatively, at FIG. 10A, computer system 600 detects user input 1050d (e.g., a tap input, a touch input, an air gesture, a mouse click, a gaze input, and/or a speech input) corresponding to account user interface object 1000a of health summary user interface 1000. In response to detecting user input 1050d, computer system 600 displays account user interface 608, as shown at FIG. 10E.

At FIG. 10B, computer system 600 displays weight user interface 1006, which includes graphical representation 1006a of weight information of the user of the account corresponding to computer system 600. Graphical representation 1006a includes a plurality of data points corresponding to weight information received by computer system 600 at respective dates. For instance, at FIG. 10B, graphical representation 1006a includes weight information for each month from April 2023 to April 2024. In some embodiments, the weight information represented by a respective data point of graphical representation 1006a includes an average of weight information for the respective month corresponding to the respective data point. In some embodiments, computer system 600 does not receive weight information at regular intervals (e.g., every day or every month). In some embodiments, computer system 600 displays graphical representation 1006a with weight information corresponding to respective times at which computer system 600 received the weight information (e.g., and does not include estimates and/or projections of weight information for times at which computer system 600 did not receive weight information).

At FIG. 10B, computer system 600 displays weight user interface 1006 when computer system 600 determines that the account corresponding to computer system 600 does not include a pregnancy designation. As set forth below with reference to FIG. 10I, computer system 600 displays additional information related to patterns and/or expected changes to the weight information on weight user interface 1006 when computer system 600 determines that the account includes a pregnancy designation.

At FIG. 10C, computer system 600 displays resting heart rate user interface 1008, which includes graphical representation 1008a of resting heart rate information of the user of the account corresponding to computer system 600. Graphical representation 1008a includes a plurality of data points corresponding to resting heart rate information received by computer system 600 at respective dates. For instance, at FIG. 10C, graphical representation 1008a includes resting heart rate information for each month from April 2023 to April 2024. In some embodiments, the resting heart rate information represented by a respective data point includes an average of resting heart rate information for the respective month corresponding to the respective data point. In some embodiments, computer system 600 does not receive resting heart rate information at regular intervals (e.g., every day or every month). In some embodiments, computer system 600 displays graphical representation 1008a with resting heart rate information corresponding to respective times at which computer system 600 received the resting heart rate information (e.g., and does not include estimates and/or projections of resting heart rate information for times at which computer system 600 did not receive resting heart rate information).

At FIG. 10C, computer system 600 displays resting heart rate user interface 1008 when computer system 600 determines that the account corresponding to computer system 600 does not include a pregnancy designation. As set forth below with reference to FIG. 10J, computer system 600 displays additional information related to patterns and/or expected changes to the resting heart rate information on resting heart rate user interface 1008 when computer system 600 determines that the account includes a pregnancy designation.

At FIG. 10D, computer system 600 displays blood pressure user interface 1010, which includes graphical representation 1010a of blood pressure information of the user of the account corresponding to computer system 600. Graphical representation 1010a includes a plurality of data points corresponding to blood pressure information received by computer system 600 at respective dates. For instance, at FIG. 10D, graphical representation 1010a includes blood pressure information for each day of a week corresponding to April 14 through April 20. In some embodiments, the blood pressure information represented by a respective data point includes an average of blood pressure information for the respective day corresponding to the respective data point. In some embodiments, computer system 600 does not receive blood pressure information at regular intervals (e.g., twice a day or once a day). In some embodiments, computer system displays graphical representation 1010a with blood pressure information corresponding to respective times at which computer system 600 received the blood pressure information (e.g., and does not include estimates and/or projections of blood pressure information for times at which computer system 600 did not receive blood pressure information).

At FIG. 10D, computer system 600 displays blood pressure user interface 1010 when computer system 600 determines that the account corresponding to computer system 600 does not include a pregnancy designation. At FIG. 10D, blood pressure user interface 1010 includes blood pressure threshold indicators 1010b-1010e, which correspond to various classifications of blood pressure ranges for people that are not pregnant. In some embodiments, computer system 600 displays and/or emphasizes respective data points on graphical representation 1010a that fall within a respective blood pressure range in response to selection of blood pressure threshold indicators 1010b-1010e. For instance, in response to detecting selection of blood pressure threshold indicator 1010b, computer system 600 highlights, bolds, enlarges, and/or emphasizes respective data points on graphical representation 1010a that correspond to a normal blood pressure range for a non-pregnant person. As set forth below with reference to FIGS. 10K-10M, computer system 600 displays different blood pressure threshold indicators for the blood pressure information when computer system 600 displays blood pressure user interface 1010 and when computer system 600 determines that the account includes a pregnancy designation.

As set forth above, in response to detecting user input 1050d corresponding to account user interface object 1000a of health summary user interface 1000, computer system 600 displays account user interface 608, as shown at FIG. 10E.

At FIG. 10E, account user interface 608 includes one or more selectable options corresponding to respective user interfaces for configuring, customizing, and/or managing the account corresponding to computer system 600. At FIG. 10E, computer system 600 detects user input 1050e (e.g., a tap input, a touch input, an air gesture, a mouse click, a gaze input, and/or a speech input) corresponding to health details user interface object 608a of account user interface 608. In response to detecting user input 1050e, computer system 600 displays health details user interface 618, as shown at FIG. 10F.

At FIG. 10F, health details user interface 618 includes information about the user of the account corresponding to computer system 600. For instance, health details user interface 618 includes date of birth indicator 618a, sex indicator 618b, height indicator 618c, and weight indicator 618d. In some embodiments, computer system 600 receives information (e.g., via one or more inputs) corresponding to the information about the user of the account corresponding to computer system 600 and displays indicators 618a-618d based on the received information. In some embodiments, indicators 618a-618d relate to information about the user of the account corresponding to computer system 600 that relate to health and/or activity of the user.

At FIG. 10F, health details user interface 618 includes add pregnancy user interface object 618e that, when selected via user input, enables computer system 600 to add a pregnancy event and/or pregnancy designation to the account. At FIG. 10F, computer system 600 detects user input 1050f (e.g., a tap input, a touch input, an air gesture, a mouse click, a gaze input, and/or a speech input) corresponding to add pregnancy user interface object 618e. In response to detecting user input 1050f, computer system 600 initiates the process for adding a pregnancy event and/or pregnancy designation to the account corresponding to computer system 600, as described above with reference to FIGS. 6G-6T.

After detecting user input 1050f, computer system 600 initiates the process to add the pregnancy designation to the account corresponding to computer system 600. After and/or while adding the pregnancy designation to the account corresponding to computer system 600, computer system 600 displays goal user interface 1012, as shown at FIG. 10G.

At FIG. 10G, goal user interface 1012 corresponds to activity goals and/or targets of the user of the account corresponding to computer system 600. Based on receiving a request to add the pregnancy designation to the account, computer system 600 displays goal user interface 1012 to enable the user to adjust, modify, and/or change the activity goals and/or targets while the user is pregnant and/or during the pregnancy event. For instance, at FIG. 10G, goal user interface 1012 includes activity summary region 1012a, which includes activity information received by computer system 600. In some embodiments, the activity information of activity summary region 1012a includes activity information received by computer system 600 within a threshold duration from a current date (e.g., within one week or within one month of the current date). In some embodiments, activity summary region 1012a provides the user with a visual indication of the activity levels achieved within the threshold duration from the current date and enables the user to determine whether to change their goals and/or targets.

At FIG. 10G, goal user interface 1012 includes change goal user interface object 1012b. In some embodiments, in response to detecting user input corresponding to change goal user interface object 1012b, computer system 600 displays a user interface that includes one or more selectable options that enable the user to input and/or select new goals and/or targets for physical activity and/or exercise. In some embodiments, in response to detecting one or more user inputs corresponding to a request to change and/or modify the goals, computer system 600 updates and/or modifies the activity goals and/or targets for the duration of the pregnancy event (e.g., and automatically changes the activity goals and/or targets back to the original goals and/or targets after the pregnancy event ends). In some embodiments, in response to detecting one or more user inputs corresponding to a request to change and/or modify the goals, computer system 600 updates and/or modifies the activity goals and/or targets and outputs a notification prompting the user to change and/or modify the goals again after the pregnancy event ends.

At FIG. 10G, goal user interface 1012 includes pause goals user interface object 1012c. In some embodiments, in response to detecting user input corresponding to pause goals user interface object 1012c, computer system 600 does not track, record, and/or receive activity information for the user. In some embodiments, in response to detecting user input selecting pause goals user interface object 1012c, computer system 600 does not track, record, and/or receive activity information for the user until receiving an additional user input requesting that computer system 600 begin tracking, recording, and/or receiving the activity information. In some embodiments, in response to detecting user input selecting pause goals user interface object 1012c, computer system 600 outputs a notification prompting the user to confirm whether to cause computer system 600 to begin tracking, recording, and/or receiving the activity information after computer system 600 determines that the pregnancy event has finished.

After displaying goal user interface 1012, computer system 600 displays (e.g., re-displays) health summary user interface 1000, as shown at FIG. 10H. At FIG. 10H, computer system 600 displays health summary user interface 1000 based on a determination that the account corresponding to computer system 600 includes the pregnancy designation (e.g., after computer system 600 added the pregnancy designation to the account).

At FIG. 10H, health summary user interface 1000 includes favorites region 1002 and trends region 1004. Favorites region 1002 includes weight platter 1002a, resting heart rate platter 1002b, and blood pressure platter 1002c. At FIG. 10H, trends region 1004 includes resting heart rate trend platter 1004b. At FIG. 10H, resting heart rate trend platter 1004b corresponds to a pattern and/or trend of resting heart rate information over time (e.g., over 18 weeks). Resting heart rate trend platter 1004b includes information indicating that the resting heart rate information received by computer system 600 includes a pattern indicative of the resting heart rate of the user increasing over the time period (e.g., over 18 weeks). In addition, computer system 600 displays trend indicator 1014 on resting heart rate trend platter 1004b based on a determination that the account corresponding to computer system 600 includes the pregnancy designation. Trend indicator 1014 includes an indication that a pattern and/or changes in resting heart rate information received during the pregnancy event may be different when compared to a pattern and/or changes in the resting heart rate information received before and/or after the pregnancy event. For instance, trend indicator 1014 includes the text explaining “This will fluctuate during pregnancy” to provide an indication to the user that variations in the resting heart rate information during the pregnancy event are based on the pregnancy event. Thus, computer system 600 provides alerts and/or indications about health information to the user so that the user is aware that their health information may vary and/or include different patterns during the pregnancy event.

At FIG. 10H, computer system 600 detects user input 1050g (e.g., a tap input, a touch input, an air gesture, a mouse click, a gaze input, and/or a speech input) corresponding to weight platter 1002a. In response to detecting user input 1050g, computer system 600 displays weight user interface 1006, as shown at FIG. 10I.

Alternatively, at FIG. 10H, computer system 600 detects user input 1050h (e.g., a tap input, a touch input, an air gesture, a mouse click, a gaze input, and/or a speech input) corresponding to resting heart rate platter 1002b. In response to detecting user input 1050h, computer system 600 displays resting heart rate user interface 1008, as shown at FIG. 10J. Alternatively, at FIG. 10H, computer system 600 detects user input 1050i (e.g., a tap input, a touch input, an air gesture, a mouse click, a gaze input, and/or a speech input) corresponding to blood pressure platter 1002c. In response to detecting user input 1050i, computer system 600 displays blood pressure user interface 1010, as shown at FIG. 10K.

At FIG. 10I, computer system 600 displays weight user interface 1006, which includes graphical representation 1006a of weight information of the user of the account corresponding to computer system 600. At FIG. 10I, computer system 600 displays weight user interface 1006 when computer system 600 determines that the account corresponding to computer system 600 includes a pregnancy designation. Based on the determination that the account includes the pregnancy designation, computer system 600 displays pregnancy timeline 1006b, trend indicator 1006c, and factors user interface object 1006d. As shown at FIG. 10B, computer system 600 does not display pregnancy timeline 1006b, trend indicator 1006c, and factors user interface object 1006d on weight user interface 1006 when computer system 600 determines that the account does not include the pregnancy designation.

At FIG. 10I, computer system 600 displays pregnancy timeline 1006b beneath graphical representation 1006a to provide a visual indication of which data points of graphical representation 1006a correspond to the pregnancy event (e.g., are representative of health information received by computer system 600 during the pregnancy event) and which data points of graphical representation 1006a do not correspond to the pregnancy event (e.g., are representative of health information received by computer system 600 before or after the pregnancy event). For instance, at FIG. 10I, pregnancy timeline 1006b includes pregnancy portion 1016a and non-pregnancy portion 1016b displayed on time axis 1016c. At FIG. 10I, time axis 1016c also corresponds to an x-axis of graphical representation 1006a. Therefore, the user of computer system 600 can quickly determine which data points included on graphical representation 1006a correspond to the pregnancy event and which data points do not correspond to the pregnancy event.

At FIG. 10I, trend indicator 1006c includes notification 1018a, which is a visual indication notifying the user of computer system 600 that patterns, relationships, and/or changes in the weight information are subject to change based on the pregnancy event (e.g., because the account corresponding to computer system 600 includes the pregnancy designation). As such, computer system 600 provides information to the user of computer system 600 that health information varies and/or changes because of the pregnancy event. For instance, pregnancy typically results in weight gain, and thus, the weight information of the user of computer system 600 is likely to increase during the pregnancy event. Thus, computer system 600 displays notification 1018a of trend indicator 1006c to inform and/or notify the user that changes in the weight information is expected because of the pregnancy event. In addition, trend indicator 1006c includes relationship indicator 1018b, which provides an indication of a pattern and/or trend of the weight information over a respective duration of time (e.g., 18 weeks). At FIG. 10I, computer system 600 displays relationship indicator 1018b to indicate that the weight information of the user has increased over the last 18 weeks.

At FIG. 10I, factors user interface object 1006d includes an indication of life factors that affect the weight information of weight user interface 1006. For instance, factors user interface object 1006d includes the text “Pregnancy” to indicate that the user is currently experiencing the pregnancy event. At FIG. 10I, factors user interface object 1006d also includes an average of the weight information. In some embodiments, the average weight information is an average of weight information during the pregnancy event. In some embodiments, the average weight information is an average of weight information for the duration indicated by time axis 1016c (e.g., one year and one month).

At FIG. 10J, computer system 600 displays resting heart rate user interface 1008, which includes graphical representation 1008a of resting heart rate information of the user of the account corresponding to computer system 600. At FIG. 10J, computer system 600 displays resting heart rate user interface 1008 when computer system 600 determines that the account corresponding to computer system 600 includes a pregnancy designation. Based on the determination that the account includes the pregnancy designation, computer system 600 displays pregnancy timeline 1008b, trend indicator 1008c, and factors user interface object 1008d. As shown at FIG. 10C, computer system 600 does not display pregnancy timeline 1008b, trend indicator 1008c, and factors user interface object 1008d on resting heart rate user interface 1008 when computer system 600 determines that the account does not include the pregnancy designation.

At FIG. 10J, computer system 600 displays pregnancy timeline 1008b beneath graphical representation 1008a to provide a visual indication of which data points of graphical representation 1008a correspond to the pregnancy event (e.g., are representative of health information received by computer system 600 during the pregnancy event) and which data points of graphical representation 1008a do not correspond to the pregnancy event (e.g., are representative of health information received by computer system 600 before or after the pregnancy event). For instance, at FIG. 10J, pregnancy timeline 1008b includes pregnancy portion 1020a and non-pregnancy portion 1020b displayed on time axis 1020c. At FIG. 10J, time axis 1020c also corresponds to an x-axis of graphical representation 1008a. Therefore, the user of computer system 600 can quickly determine which data points included on graphical representation 1008a correspond to the pregnancy event and which data points do not correspond to the pregnancy event.

At FIG. 10J, trend indicator 1008c includes notification 1022a, which is a visual indication notifying the user of computer system 600 that patterns, relationships, and/or changes in the resting heart rate information are subject to change based on the pregnancy event (e.g., because the account corresponding to computer system 600 includes the pregnancy designation). As such, computer system 600 provides information to the user of computer system 600 that health information varies and/or changes because of the pregnancy event. For instance, pregnancy typically results in an increased resting heart rate, and thus, the resting heart rate information of the user of computer system 600 is likely to increase during the pregnancy event. Thus, computer system 600 displays notification 1022a of trend indicator 1008c to inform and/or notify the user that changes in the resting heart rate information is expected because of the pregnancy event. In addition, trend indicator 1008c includes relationship indicator 1022b, which provides an indication of a pattern and/or trend of the resting heart rate information over a respective duration of time (e.g., 18 weeks). At FIG. 10J, computer system 600 displays relationship indicator 1022b to indicate that the resting heart rate information of the user has increased over the last 18 weeks.

At FIG. 10J, factors user interface object 1008d includes an indication of life factors that affect the resting heart rate information of resting heart rate user interface 1008. For instance, factors user interface object 1008d includes the text “Pregnancy” to indicate that the user is currently experiencing the pregnancy event. At FIG. 10J, factors user interface object 1008d also includes an average of the resting heart rate information. In some embodiments, the average resting heart rate information is an average of resting heart rate information during the pregnancy event. In some embodiments, the average resting heart rate information is an average of resting heart rate information for the duration indicated by time axis 1020c (e.g., one year and one month).

At FIG. 10K, computer system 600 displays blood pressure user interface 1010, which includes graphical representation 1010a of blood pressure information of the user of the account corresponding to computer system 600. At FIG. 10K, computer system 600 displays blood pressure user interface 1010 when computer system 600 determines that the account corresponding to computer system 600 includes a pregnancy designation. Based on the determination that the account includes the pregnancy designation, computer system 600 displays pregnancy timeline 1010f and pregnancy blood pressure threshold indicators 1010g-1010i. As shown at FIG. 10D, computer system 600 does not display pregnancy timeline 1010f and/or pregnancy blood pressure threshold indicators 1010g-1010i on blood pressure user interface 1010 when computer system 600 determines that the account does not include the pregnancy designation.

At FIG. 10K, graphical representation 1010a includes a plurality of data points corresponding to blood pressure information received by computer system 600 at respective dates and/or times. For instance, at FIG. 10K, graphical representation 1010a includes blood pressure information for each day of a week corresponding to April 14 through April 20. In some embodiments, the blood pressure information represented by a respective data point includes an average of blood pressure information for the respective day corresponding to the respective data point. In some embodiments, computer system 600 does not receive blood pressure information at regular intervals. In some embodiments, computer system displays graphical representation 1010a with blood pressure information corresponding to respective times at which computer system 600 received the blood pressure information (e.g., and does not include estimates and/or projections of blood pressure information for times at which computer system 600 did not receive blood pressure information).

At FIG. 10K, computer system 600 displays pregnancy timeline 1010f beneath graphical representation 1010a to provide a visual indication of which data points of graphical representation 1010a correspond to the pregnancy event (e.g., are representative of health information received by computer system 600 during the pregnancy event) and which data points of graphical representation 1010a do not correspond to the pregnancy event (e.g., are representative of health information received by computer system 600 before or after the pregnancy event). For instance, at FIG. 10K, pregnancy timeline 1010f includes pregnancy portion 1024a and time axis 1024b. At FIG. 10K, pregnancy timeline 1010f does not include a non-pregnancy portion because computer system 600 determines that the pregnancy event occurred throughout the duration represented by time axis 1024b. In some embodiments, when computer system 600 determines that the pregnancy event did not occur during at least some of the duration represented by time axis 1024b, computer system 600 displays pregnancy timeline 1010f with a non-pregnancy portion. At FIG. 10K, time axis 1020c also corresponds to an x-axis of graphical representation 1010a. Therefore, the user of computer system 600 can quickly determine which data points included on graphical representation 1010a correspond to the pregnancy event and which data points do not correspond to the pregnancy event.

At FIG. 10K, blood pressure user interface 1010 includes pregnancy blood pressure threshold indicators 1010g-1010i in addition to blood pressure threshold indicators 1010b-1010e. As set forth above, blood pressure threshold indicators 1010b-1010e correspond to various classifications of blood pressure ranges for people that are not pregnant. At FIG. 10K, pregnancy blood pressure threshold indicators 1010g-1010i correspond to various classifications of blood pressure ranges for people that are pregnant. In some embodiments, computer system 600 forgoes displaying blood pressure threshold indicators 1010b-1010e on blood pressure user interface 1010 based on a determination that the pregnancy event occurred throughout the duration of time axis 1024b (e.g., computer system 600 determines that no data points currently displayed on graphical representation 1010a correspond to a time period when the pregnancy event was not occurring). In some embodiments, computer system 600 displays blood pressure threshold indicators 1010b-1010e with a grayed-out and/or darkened appearance to indicate that the data points currently displayed on graphical representation 1010a correspond to the pregnancy event (e.g., no data points currently displayed on graphical representation 1010a correspond to a time period outside of the pregnancy event).

In some embodiments, computer system 600 displays and/or emphasizes respective data points on graphical representation 1010a that fall within a respective blood pressure range in response to selection of pregnancy blood pressure threshold indicators 1010g-1010i. For instance, at FIG. 10K, computer system 600 displays pregnancy blood pressure threshold indicator 1010g with an appearance (e.g., as indicated by bold text at FIG. 10K) indicating that pregnancy blood pressure threshold indicator 1010g is currently selected. At FIG. 10K, computer system 600 highlights, bolds, enlarges, and/or emphasizes respective data points on graphical representation 1010a that correspond to a normal blood pressure range for a pregnant person.

At FIG. 10K, computer system 600 detects user input 1050j (e.g., a tap input, a touch input, an air gesture, a mouse click, a gaze input, and/or a speech input) corresponding to pregnancy blood pressure threshold indicator 1010h. In response to detecting user input 1050j, computer system 600 displays blood pressure user interface 1010, as shown at FIG. 10L.

At FIG. 10L, computer system 600 displays pregnancy blood pressure threshold indicator 1010h with an appearance (e.g., as indicated by bold text at FIG. 10L) indicating that pregnancy blood pressure threshold indicator 1010h is currently selected. At FIG. 10L, computer system 600 highlights, bolds, enlarges, and/or emphasizes respective data points on graphical representation 1010a that correspond to a mildly elevated blood pressure range for a pregnant person. Therefore, computer system 600 is configured to quickly organize, sort, and/or arrange the blood pressure information of blood pressure user interface 1010 based on user input corresponding to pregnancy blood pressure threshold indicators 1010g-1010i and/or blood pressure threshold indicators 1010b-1010e.

At FIG. 10L, computer system 600 detects user input 1050k (e.g., a tap input, a touch input, an air gesture, a mouse click, a gaze input, and/or a speech input) corresponding to pregnancy blood pressure threshold indicator 1010i. In response to detecting user input 1050k, computer system 600 displays blood pressure user interface 1010, as shown at FIG. 10M.

At FIG. 10M, computer system 600 displays pregnancy blood pressure threshold indicator 1010i with an appearance (e.g., as indicated by bold text at FIG. 10M) indicating that pregnancy blood pressure threshold indicator 1010i is currently selected. At FIG. 10M, computer system 600 determines that none of the data points currently displayed on graphical representation 1010a correspond to blood pressure information that falls within the severely elevated blood pressure threshold range. As such, at FIG. 10M, computer system 600 forgoes highlighting, bolding, enlarging, and/or emphasizing data points on graphical representation 1010a. In some embodiments, in response to detecting user input 1050k, computer system 600 deemphasizes (e.g., decreases a size of, reduces a brightness of, and/or changes a color of) data points of graphical representation 1010a that correspond to a normal blood pressure range for a pregnant person and/or data points of graphical representation 1010a that correspond to a mildly elevated blood pressure range for a pregnant person.

At FIG. 10M, computer system 600 detects user input 10501 (e.g., a tap input, a touch input, an air gesture, a mouse click, a gaze input, and/or a speech input) corresponding to done user interface object 1010j of blood pressure user interface 1010. In response to detecting user input 10501, computer system 600 displays (e.g., re-displays) health summary user interface 1000, as shown at FIG. 10N.

At FIG. 10N, computer system 600 detects user input 1050m (e.g., a tap input, a touch input, an air gesture, a mouse click, a gaze input, and/or a speech input) corresponding to all trends user interface object 1004c of trend region 1004 of health summary user interface 1000. In response to detecting user input 1050m, computer system 600 displays trend user interface 1026, as shown at FIG. 10O.

At FIG. 10O, computer system 600 displays trend user interface 1026 when computer system 600 determines that the account corresponding to computer system 600 includes a pregnancy designation. At FIG. 10O, trend user interface 1026 includes first trend region 1028 corresponding to health information that computer system 600 determines has improved over a respective duration of time. Trend user interface 1026 includes second trend region 1030 corresponding to health information in which computer system 600 determines has regressed and/or stayed relatively stable over a respective duration of time. Further, trend user interface 1026 includes information region 1026a including a visual indication (e.g., text) notifying the user of computer system 600 to consult with their doctor and/or care team about exercising during the pregnancy event.

At FIG. 10O, first trend region 1028 includes move platter 1028a corresponding to a pattern and/or change in the amount of calories burned by the user of computer system 600 per day. At FIG. 10O, computer system 600 determines that the amount of calories burned per day by the user has increased, and displays move platter 1028a in first trend region.

At FIG. 10O, second trend region 1030 includes cardio fitness platter 1030a, exercise platter 1030b, and distance platter 1030c. In some embodiments, computer system 600 receives information related to the cardio fitness, exercise minutes, and distance traveled by the user of computer system 600 over time and determines that the information indicates that the cardio fitness, exercise minutes, and distance traveled by the user have each decreased over a respective duration of time. At FIG. 10O, computer system 600 displays cardio fitness platter 1030a with trend indicator 1032. Trend indicator 1032 provides information and/or an indication that patterns, relationships, and/or changes in the cardio fitness of the user are expected during the pregnancy event. For instance, at FIG. 10O, trend indicator 1032 includes text indicating that a reduction in cardio fitness is expected during pregnancy. As such, computer system 600 provides an indication that the cardio fitness information of the user is expected to decrease when computer system 600 determines that the account includes the pregnancy designation. In some embodiments, computer system 600 displays trend indicators with exercise platter 1030b and distance platter 1030c.

At FIG. 10O, computer system 600 detects user input 1050n (e.g., a tap input, a touch input, an air gesture, a mouse click, a gaze input, and/or a speech input) corresponding to cardio fitness platter 1030a. In response to detecting user input 1050n, computer system 600 displays cardio fitness user interface 1034, as shown at FIG. 10P.

At FIG. 10P, cardio fitness user interface 1034 includes additional information related to cardio fitness information of the user received and/or determined by computer system 600. At FIG. 10P, computer system 600 displays cardio fitness user interface 1034 when computer system 600 determines that the account corresponding to computer system 600 includes a pregnancy designation. At FIG. 10P, cardio fitness user interface 1034 includes trend indicator 1034a indicating that patterns, relationships, and/or changes in the cardio fitness of the user are expected during the pregnancy event. For instance, at FIG. 10P, trend indicator 1032 includes text indicating that a reduction in cardio fitness is expected during pregnancy.

At FIG. 10P, cardio fitness user interface 1034 also includes graphical representation 1034b of cardio fitness information received by and/or determined by computer system 600. As shown at FIG. 10P, graphical representation 1034b indicates that the cardio fitness information of the user of computer system 600 decreased over a respective duration of time (e.g., one year and one month).

FIG. 11 is a flow diagram illustrating a method for tracking health information during a pregnancy event using a computer system in accordance with some embodiments. Method 1100 is performed at a computer system (e.g., 100, 300, 500, and/or 600) (e.g., a smart phone, a smart watch, a tablet computer, a laptop computer, a desktop computer, a wearable device, and/or a head-mounted device) that is in communication with one or more display generation components (e.g., 602) (e.g., one or more displays, touch-screen displays, monitors, holographic display systems, and/or head-mounted display systems) and one or more input devices (e.g., 602) (e.g., a touch-sensitive surface (e.g., a touch-sensitive display); a mouse; a keyboard; a remote control; a visual input device (e.g., one or more cameras such as, e.g., an infrared camera, a depth camera, a visible light camera, and/or a gaze tracking camera); an audio input device (e.g., a microphone); a biometric sensor (e.g., a fingerprint sensor, a face identification sensor, a gaze tracking sensor, and/or an iris identification sensor); and/or one or more mechanical input devices (e.g., a depressible input mechanism; a button; a rotatable input mechanism; a crown; and/or a dial)). Some operations in method 1100 are, optionally, combined, the orders of some operations are, optionally, changed, and some operations are, optionally, omitted.

As described below, method 1100 provides an intuitive way for tracking health information during a pregnancy event. The method reduces the cognitive burden on a user for tracking health information during a pregnancy event, thereby creating a more efficient human-machine interface. For battery-operated computing devices, enabling a user to track health information during a pregnancy event faster and more efficiently conserves power and increases the time between battery charges.

The computer system (e.g., 100, 300, 500, and/or 600) detects (1102), via the one or more input devices, user input (e.g., 1050a, 1050b, 1050c, 1050g, 1050h, 1050i, 1050m, and/or 1050n) (e.g., a tap gesture, a swipe gesture, a touch gesture, an air gesture, a button press, and/or a voice command) corresponding to a request to display a user interface (e.g., 1006, 1008, 1010, 1026, and/or 1034) corresponding to health information (e.g., a user interface associated with a health application, an activity application, and/or a fitness application that includes information related to health of a user, such as heart rate information, number of steps taken over a respective duration, number of calories burned over a respective duration, walking and/or running distance over a respective duration, number of flights of stairs climbed over a respective duration, an indication of an amount of time standing over a respective duration, a blood oxygen percentage, walking speed, walking length, cardio fitness, time in daylight over a respective duration, walking steadiness, and/or sleeping information) of an account (e.g., an account and/or user profile of a user of the computer system that is accessible by the computer system) of the computer system (e.g., 100, 300, 500, and/or 600).

In response to detecting the user input (e.g., 1050a, 1050b, 1050c, 1050g, 1050h, 1050i, 1050m, and/or 1050n) corresponding to the request to display the user interface (e.g., 1006, 1008, 1010, 1026, and/or 1034) corresponding to health information of the account of the computer system (e.g., 100, 300, 500, and/or 600), the computer system (e.g., 100, 300, 500, and/or 600) displays (1104), via the one or more display generation components (e.g., 602), the user interface (e.g., 1006, 1008, 1010, 1026, and/or 1034) corresponding to health information of the account of the computer system (e.g., 100, 300, 500, and/or 600).

Displaying the user interface (e.g., 1006, 1008, 1010, 1026, and/or 1034) includes the computer system (e.g., 100, 300, 500, and/or 600) displaying, via the one or more display generation components, a graphical representation (1106) (e.g., 1006a, 1008a, 1010a, and/or 1034b) (e.g., a chart, grid, and/or graph that includes a visual representation of health information over time) of first health data (e.g., heart rate information, number of steps taken over a respective duration, number of calories burned over a respective duration, walking and/or running distance over a respective duration, number of flights of stairs climbed over a respective duration, an indication of an amount of time standing over a respective duration, a blood oxygen percentage, walking speed, walking length, cardio fitness, time in daylight over a respective duration, walking steadiness, and/or sleeping information) corresponding to a first time duration (e.g., a first range of time that occurred prior to a current time, such as the current day up to the current time, the prior week and/or the current week up to the current time, the prior month and/or the current month up to the current time, and/or the prior year and/or the current year up to the current time). In some embodiments, the first time duration includes a period of time when a user of the account of the computer system (e.g., 100, 300, 500, and/or 600) is not pregnant (e.g., the computer system has not received information and/or an indication that the user of the account is pregnant) and/or the computer system (e.g., 100, 300, 500, and/or 600) does not include information about a pregnancy status of the user of the account of the computer system (e.g., 100, 300, 500, and/or 600).

Displaying the user interface (e.g., 1006, 1008, 1010, 1026, and/or 1034) includes: in accordance with a determination that the account of the computer system (e.g., 100, 300, 500, and/or 600) includes an indication that a user of the account is pregnant (e.g., the account includes a designation, such as a user-defined designation received via one or more user inputs, indicating that the user of the account is currently pregnant and/or was pregnant within a threshold time range of a current day), the computer system (e.g., 100, 300, 500, and/or 600) displaying (1108), via the one or more display generation components (e.g., 602), an indication (e.g., 1014, 1018a, 1022a, 1032, and/or 1034a) (e.g., text, images, icons, photos, and/or graphical user interface objects) that a difference between second health data (e.g., heart rate information, number of steps taken over a respective duration, number of calories burned over a respective duration, walking and/or running distance over a respective duration, number of flights of stairs climbed over a respective duration, an indication of an amount of time standing over a respective duration, a blood oxygen percentage, walking speed, walking length, cardio fitness, time in daylight over a respective duration, walking steadiness, and/or sleeping information) and the first health data is at least partially based on (e.g., caused by and/or a result of) the user of the account being pregnant (e.g., the indication provides visual feedback to the user of the computer system that the health information may change as a result of the user being pregnant, which may include, for example, changes in trends of activity information and/or changes in health information detected via one or more input devices of the computer system), wherein the second health data corresponds to a second time duration (e.g., a second range of time that includes the time at which the user of the computer system is pregnant) that is after (e.g., later than) the first time duration. In some embodiments, in accordance with a determination that the account of the computer system (e.g., 100, 300, 500, and/or 600) does not include an indication that the user of the account is pregnant (e.g., the account does not includes a designation, such as a user-defined designation received via one or more user inputs, indicating that the user of the account is currently pregnant and/or was pregnant within a threshold time range of a current day), the computer system (e.g., 100, 300, 500, and/or 600) forgoes displaying the indication that the difference between the second health data and the first health data is at least partially based on the user of the account being pregnant.

Displaying the indication that a difference between second health data and the first health data is at least partially based on the user of the account being pregnant in accordance with a determination that the account of the computer system includes an indication that the user of the account is pregnant allows a user to determine that the health data and/or health information changes based on the user being pregnant, thereby providing improved visual feedback to the user.

In some embodiments, displaying the user interface (e.g., 1006, 1008, 1010, 1026, and/or 1034) includes: in accordance with the determination that the account of the computer system (e.g., 100, 300, 500, and/or 600) includes the indication that the user of the account is pregnant, the computer system (e.g., 100, 300, 500, and/or 600) displaying, via the one or more display generation components (e.g., 602), a first threshold (e.g., 1010g-1010i) (e.g., a first value of a respective health parameter in which the computer system compares received health data corresponding to the health parameter to determine whether the received health data indicates that the user is experiencing elevated and/or lower levels of the health parameter when compared to what is considered normal levels of the health parameter) corresponding to the health information; and in accordance with a determination that the account of the computer system (e.g., 100, 300, 500, and/or 600) does not include an indication that the user of the account is pregnant, the computer system (e.g., 100, 300, 500, and/or 600) displaying, via the one or more display generation components (e.g., 602), a second threshold (e.g., 1010b-1010e) (e.g., a second value of a respective health parameter in which the computer system compares received health data corresponding to the health parameter to determine whether the received health data indicates that the user is experiencing elevated and/or lower levels of the health parameter when compared to what is considered normal levels of the health parameter) corresponding to the health information, wherein the second threshold (e.g., 1010b-1010e) is different from the first threshold (e.g., 1010g-1010i). In some embodiments, the first threshold (e.g., 1010g-1010i) is determined to be a first value that is based on what is considered a normal value for a pregnant person. In some embodiments, the second threshold (e.g., 1010b-1010e) is determined to be a second value that is based on what is considered a normal value for a non-pregnant person. Displaying the first threshold corresponding to the health information in accordance with the determination that the account of the computer system includes the indication that the user of the account is pregnant and displaying the second threshold corresponding to the health information in accordance with the determination that the account of the computer system does not include an indication that the user of the account is pregnant provides a user with a visual indication of whether or not the health information of the user is considered normal based on whether the user is pregnant or not, thereby providing improved visual feedback to the user.

In some embodiments, displaying the user interface (e.g., 1006, 1008, 1010, 1026, and/or 1034) includes: in accordance with the determination that the account of the computer system (e.g., 100, 300, 500, and/or 600) includes the indication that the user of the account is pregnant, the computer system (e.g., 100, 300, 500, and/or 600) displaying, via the one or more display generation components, one or more factors (e.g., 1006d and/or 1008d) (e.g., conditions and/or characteristics that affect the health and/or health information of a user, such as whether the user is pregnant or not, whether the user is experiencing lactation or not, whether the user is using a contraceptive or not, and/or whether the user is experiencing an illness or not) that affect the health information; and in accordance with a determination that the account of the computer system (e.g., 100, 300, 500, and/or 600) does not include an indication that the user of the account is pregnant, the computer system (e.g., 100, 300, 500, and/or 600) forgoing (e.g., not displaying) display of the one or more factors (e.g., 1006d and/or 1008d) that affect the health information. Displaying the one or more factors in accordance with the determination that the account of the computer system includes the indication that the user of the account is pregnant and forgoing displaying the one or more factors in accordance with the determination that the account of the computer system does not include an indication that the user of the account is pregnant enables a pregnant user to quickly determine whether additional factors are contributing to the health information, thereby providing improved visual feedback to the user.

In some embodiments, displaying the user interface (e.g., 1006, 1008, 1010, 1026, and/or 1034) includes: in accordance with the determination that the account of the computer system (e.g., 100, 300, 500, and/or 600) includes the indication that the user of the account is pregnant, the computer system (e.g., 100, 300, 500, and/or 600) displaying, via the one or more display generation components (e.g., 602), a timeline (e.g., 1006b, 1008b, and/or 1010f) of a pregnancy of the user of the account (e.g., a graphical user interface object that visually indicates a start date of the pregnancy, a completion date and/or estimated due date of the pregnancy, and/or a current date, such a as a progress bar and/or line); and in accordance with a determination that the account of the computer system (e.g., 100, 300, 500, and/or 600) does not include an indication that the user of the account is pregnant, the computer system (e.g., 100, 300, 500, and/or 600) forgoing (e.g., not displaying) display of the timeline (e.g., 1006b, 1008b, and/or 1010f) of the pregnancy of the user of the account. Displaying the time of the pregnancy of the user of the account in accordance with the determination that the account of the computer system includes the indication that the user of the account is pregnant and forgoing displaying the timeline of the pregnancy of the user of the account in accordance with a determination that the account of the computer system does not include an indication that the user of the account is pregnant allows the user to quickly determine whether the user is pregnant or not and how far along the pregnancy is relative to a current date if the user is pregnant, thereby providing improved visual feedback to the user.

In some embodiments, while displaying the indication (e.g., 1014, 1018a, 1022a, 1032, and/or 1034a) that the difference between the second health data and the first health data is at least partially based on the user of the account being pregnant, the computer system (e.g., 100, 300, 500, and/or 600) receives (e.g., via one or more user inputs and/or via one or more input devices of the computer system) information (e.g., blood pressure at FIG. 10L) indicating that a health data point (e.g., a measured and/or received value of a respective health parameter at a respective time and/or date) of the health information is above a threshold value (e.g., 1010h) (e.g., the health data point is above a value of a respective health parameter that is considered a normal level of the health parameter, thereby indicating that the user is experiencing elevated levels of the respective health parameter). In response to receiving the information (e.g., blood pressure at FIG. 10L) indicating that the health data point of the health information is above the threshold value (e.g., 1010h), the computer system (e.g., 100, 300, 500, and/or 600) outputs an indication (e.g., “1 DAY” shown at 1010h) (e.g., text, an alert, a notification, a user interface, one or more user interface objects, audio, and/or tactile feedback) that the health data point of the health information is above the threshold value (e.g., 1010h) (e.g., displaying and/or otherwise providing information to the user indicating the health data point is above the threshold value, thereby indicating that the health data point is outside of a range of values that is considered to be normal for the respective health parameter). Outputting the indication that the health data point of the health information is above the threshold value in response to receiving the information indicating that the health data point of the health information is above the threshold value provides the user with information that the health data point is above the threshold value, thereby providing improved visual feedback to the user and/or performing an operation when a set of conditions has been met without requiring further user input.

In some embodiments, displaying the user interface (e.g., 1006, 1008, 1010, 1026, and/or 1034) includes: in accordance with the determination that the account of the computer system (e.g., 100, 300, 500, and/or 600) includes the indication that the user of the account is pregnant and in accordance with a determination that a current date (e.g., a date in which the computer system is displaying the user interface) is within a threshold duration (e.g., within one day, within one week, within two weeks, or within one month) of a due date (e.g., 614b) of a pregnancy of the user of the account (e.g., an estimated due date of the pregnancy of the user of the account, such as an estimated, predicted, and/or projected date at which the user corresponding to the account of the computer system will and/or is likely to give birth, such as a date provided to the user by a physician and/or doctor and/or a date estimated by the user), the computer system (e.g., 100, 300, 500, and/or 600) displaying, via the one or more display generation components (e.g., 602), an indication (e.g., text, a user interface, and/or one or more user interface objects) that a difference between third health data (e.g., heart rate information, number of steps taken over a respective duration, number of calories burned over a respective duration, walking and/or running distance over a respective duration, number of flights of stairs climbed over a respective duration, an indication of an amount of time standing over a respective duration, a blood oxygen percentage, walking speed, walking length, cardio fitness, time in daylight over a respective duration, walking steadiness, and/or sleeping information) and the second health data is at least partially based on (e.g., caused by and/or a result of) the user of the account giving birth (e.g., delivering a baby and/or the pregnancy of the user of the account ending), wherein the third health data corresponds to a third time duration (e.g., a third range of time that includes the time after the due date of the pregnancy of the user of the account) that is after (e.g., later than) the second time duration. Displaying the indication that a difference between third health data and the second health data is at least partially based on the user of the account giving birth in accordance with a determination that the current date is within a threshold duration of the due date of the pregnancy of the user of the account allows a user to determine that the health data and/or health information changes based on the pregnancy of the user being completed, thereby providing improved visual feedback to the user.

In some embodiments, displaying the user interface (e.g., 1006, 1008, 1010, 1026, and/or 1034) includes: in accordance with the determination that the account of the computer system (e.g., 100, 300, 500, and/or 600) includes the indication that the user of the account is pregnant, the computer system (e.g., 100, 300, 500, and/or 600) displaying, via the one or more display generation components (e.g., 602), one or more user interface objects (e.g., 1010b) (e.g., one or more buttons, affordances, graphical user interface elements, user selectable icons, menus, and/or regions of a display generation component of the one or more display generation components) for adjusting (e.g., changing and/or modifying) a health goal (e.g., a target value of a respective health parameter in which the user of the account intends to achieve over a respective amount of time, such as one day, one month, and/or one year) corresponding to the health information. Displaying the one or more user interface objects for adjusting the health goal corresponding to the health information in accordance with the determination that the account of the computer system includes the indication that the user of the account is pregnant allows a user to quickly change the health goal without requiring the user to provide additional input and/or without requiring the user to navigate to a different user interface, thereby reducing the number of inputs needed to perform an operation and/or performing an operation when a set of conditions has been met without requiring further user input.

Note that details of the processes described above with respect to method 1100 (e.g., FIG. 11) are also applicable in an analogous manner to the methods described above. For example, methods 700 and/or 900 optionally include one or more of the characteristics of the various methods described above with reference to method 1100. For example, the computer system of method 700 can be used to add a pregnancy designation to an account of the computer systems of methods 900 and 1100. For brevity, these details are not repeated below.

The foregoing description, for purpose of explanation, has been described with reference to specific embodiments. However, the illustrative discussions above are not intended to be exhaustive or to limit the invention to the precise forms disclosed. M any modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying drawings, it is to be noted that various changes and modifications will become apparent to those skilled in the art. Such changes and modifications are to be understood as being included within the scope of the disclosure and examples as defined by the claims.

As described above, one aspect of the present technology is the gathering and use of data available from various sources to improve visual feedback to users about a pregnancy event. The present disclosure contemplates that in some instances, this gathered data may include personal information data that uniquely identifies or can be used to contact or locate a specific person. Such personal information data can include demographic data, location-based data, telephone numbers, email addresses, social network IDs, home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to display health information and/or information about a pregnancy event. Accordingly, use of such personal information data enables users to view information relevant to their health. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of health information, the present technology can be configured to allow users to select to “opt in” or “opt out” of participation in the collection of personal information data during registration for services or anytime thereafter. In another example, users can select not to provide health information. In yet another example, users can select to limit the length of time health information is maintained or entirely prohibit the collection of health information. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, health user interfaces can be displayed based on non-personal information data or a bare minimum amount of personal information, such as the content being requested by the device associated with a user, other non-personal information, or publicly available information.

1-62. (canceled)

63. A computer system configured to communicate with one or more display generation components and one or more input devices, comprising:
one or more processors; and
memory storing one or more programs configured to be executed by the one or more processors, the one or more programs including instructions for:
detecting, via the one or more input devices, one or more user inputs corresponding to a request to add a pregnancy event to health information of an account of the computer system;
in response to detecting the one or more user inputs corresponding to the request to add the pregnancy event to the health information of the account of the computer system, displaying, via the one or more display generation components, a pregnancy user interface that includes a pregnancy estimation user interface object;
detecting, via the one or more input devices, one or more user inputs corresponding to the pregnancy estimation user interface object;
in response to detecting the one or more user inputs corresponding to the pregnancy estimation user interface object, receiving information about an age of the pregnancy event; and
after receiving the information about the age of the pregnancy event, displaying, via the one or more display generation components, one or more user interfaces corresponding to options to configure respective notifications related to the pregnancy event.

64. The computer system of claim 63, wherein the information about the age of the pregnancy event includes one or more of: a last menstrual period, an approximate due date of the pregnancy event, an approximate gestational age of the pregnancy event, and a date of an embryo transfer.

65. The computer system of claim 63, the one or more programs further including instructions for:
in response to receiving the information about the age of the pregnancy event and prior to displaying the one or more user interfaces corresponding to options to configure respective notifications related to the pregnancy event, displaying, via the one or more display generation components, estimated pregnancy information that is based on the information about the age of the pregnancy event.

66. The computer system of claim 65, wherein the estimated pregnancy information includes an estimated pregnancy age.

67. The computer system of claim 65, wherein the estimated pregnancy information includes an estimated due date.

68. The computer system of claim 63, wherein the one or more user interfaces corresponding to options to configure respective notifications related to the pregnancy event include a walking steadiness user interface corresponding to options to configure walking steadiness notifications.

69. The computer system of claim 63, wherein the one or more user interfaces corresponding to options to configure respective notifications related to the pregnancy event include a cardio fitness user interface corresponding to options to configure cardio fitness notifications.

70. The computer system of claim 63, wherein the one or more user interfaces corresponding to options to configure respective notifications related to the pregnancy event include a mental health user interface corresponding to options to configure mental health notifications.

71. The computer system of claim 63, the one or more programs further including instructions for:
after displaying the one or more user interfaces corresponding to options to configure respective notifications related to the pregnancy event, displaying, via the one or more display generation components, a medication user interface.

72. The computer system of claim 71, the one or more programs further including instructions for:
while displaying the medication user interface, detecting, via the one or more input devices, user input corresponding to a request to add a medication to the health information of the account of the computer system; and
in response to detecting the user input corresponding to the request to add the medication to the health information of the account of the computer system, displaying, via the one or more display generation components, information about the medication.

73. The computer system of claim 71, the one or more programs further including instructions for:
while displaying the medication user interface, detecting, via the one or more input devices, user input corresponding to a request to add a second medication to the health information of the account of the computer system; and
in response to detecting the user input corresponding to the request to add the second medication to the health information of the account of the computer system:
in accordance with a determination that the second medication satisfies a set of criteria, outputting an alert corresponding to the second medication.

74. The computer system of claim 63, the one or more programs further including instructions for:
after displaying the one or more user interfaces corresponding to options to configure respective notifications related to the pregnancy event, displaying, via the one or more display generation components, a health settings user interface, wherein the health settings user interface includes a user interface object corresponding to an option to change an existing health setting;
while displaying the health settings user interface, detecting, via the one or more input devices, user input corresponding to the health settings user interface; and
in response to detecting the user input corresponding to the health settings user interface:
in accordance with a determination that the user input corresponds to the user interface object corresponding to the option to change the existing health setting, adjusting the existing health setting.

75. The computer system of claim 74, wherein the user interface object corresponding to the option to change the existing health setting corresponds to an option to change a threshold heart rate.

76. The computer system of claim 74, wherein the user interface object corresponding to the option to change the existing health setting corresponds to an option to change a status of a walking steadiness feature.

77. The computer system of claim 74, wherein the user interface object corresponding to the option to change the existing health setting corresponds to an option to change a status of a cardio fitness feature.

78. The computer system of claim 74, wherein the user interface object corresponding to the option to change the existing health setting corresponds to an option to add a pregnancy designation to a medical profile of the account of the computer system.

79. The computer system of claim 63, the one or more programs further including instructions for:
after displaying the one or more user interfaces corresponding to options to configure respective notifications related to the pregnancy event, adding the pregnancy event to the health information of the account of the computer system; and
in response to detecting that a current date is within a threshold duration of an estimated due date of the pregnancy event, displaying, via the one or more display generation components, a pregnancy completion user interface, wherein the pregnancy completion user interface corresponds to an option to remove the pregnancy event from the health information of the account of the computer system.

80. The computer system of claim 79, wherein the pregnancy completion user interface includes a user interface object corresponding to an option to initiate a lactation tracking feature.

81. The computer system of claim 79, wherein the pregnancy completion user interface includes a second user interface object corresponding to an option to remove a pregnancy designation from a medical profile of the account of the computer system.

82. A non-transitory computer-readable storage medium storing one or more programs configured to be executed by one or more processors of a computer system that is in communication with one or more display generation components and one or more input devices, the one or more programs including instructions for:
detecting, via the one or more input devices, one or more user inputs corresponding to a request to add a pregnancy event to health information of an account of the computer system;
in response to detecting the one or more user inputs corresponding to the request to add the pregnancy event to the health information of the account of the computer system, displaying, via the one or more display generation components, a pregnancy user interface that includes a pregnancy estimation user interface object;
detecting, via the one or more input devices, one or more user inputs corresponding to the pregnancy estimation user interface object;
in response to detecting the one or more user inputs corresponding to the pregnancy estimation user interface object, receiving information about an age of the pregnancy event; and
after receiving the information about the age of the pregnancy event, displaying, via the one or more display generation components, one or more user interfaces corresponding to options to configure respective notifications related to the pregnancy event.

83. A method, comprising:
at a computer system that is in communication with one or more display generation components and one or more input devices:
detecting, via the one or more input devices, one or more user inputs corresponding to a request to add a pregnancy event to health information of an account of the computer system;
in response to detecting the one or more user inputs corresponding to the request to add the pregnancy event to the health information of the account of the computer system, displaying, via the one or more display generation components, a pregnancy user interface that includes a pregnancy estimation user interface object;
detecting, via the one or more input devices, one or more user inputs corresponding to the pregnancy estimation user interface object;
in response to detecting the one or more user inputs corresponding to the pregnancy estimation user interface object, receiving information about an age of the pregnancy event; and
after receiving the information about the age of the pregnancy event, displaying, via the one or more display generation components, one or more user interfaces corresponding to options to configure respective notifications related to the pregnancy event.
